(19) United States
(12) Patent Application Publication
(10) Pub. No.: US 2010/0203521 A1
(43) Pub. Date: Aug. 12, 2010

(54) METHOD FOR BACTERIAL LYSIS

(75) Inventors: Catherine M. Klapperich, Boston, MA (US); Jessica Dare Kaufman, Woburn, MA (US); Maria Dominika Kulinski, Danvers, MA (US); David Altman, Framingham, MA (US); Satish Singh, Sharon, MA (US)

Correspondence Address: RONALD I. EISENSTEIN, 100 SUMMER STREET, NIXON PEABODY LLP, BOSTON, MA, 02110, US

(73) Assignee: BOSTON MEDICAL CENTER CORPORATION, Boston, MA (US)

(21) Appl. No.: 12/594,427

(22) PCT Filed: Apr. 2, 2008
(86) PCT No.: PCT/US08/59158

§ 371 Date: Apr. 23, 2010

Related U.S. Application Data
(60) Provisional application No. 60/921,404, filed on Apr. 2, 2007.
(60) Provisional application No. 60/925,445, filed on Apr. 20, 2007.

(51) Int. Cl. C12Q 1/68; C12M 1/34

(52) U.S. Cl. 435/6; 435/287.2; 977/742

(57) ABSTRACT

The present invention is directed to a microfluidic device for lysis of cells, such as bacteria and microorganisms. In particular, the present invention relates to microfluidic devices and methods of manufacture of such microfluidic devices comprising a substrate with at least one channel packed with a polymer monolith embedded with carbon particles, for example carbon nanotubes. The microfluidic devices and methods of the present invention are useful for cell lysis of cells within a biological sample, such as a untreated biological sample comprising microorganisms, such as but not limited to gram positive and gram negative bacteria. In some embodiments, the microfluidic devices of the present invention can also optionally comprise other modules enabling further processing of the biological sample, for example isolation, purification and detection of biomolecules released from the lysed cells, such as but not limited to nucleic acids or proteins or peptides from the lysed cells, providing a complete Lab-on-a-Chip analysis system for biomolecules released from difficult to lyse microorganisms in a single step or process. The microfluidic devices of the present invention can also be adapted and are useful to methods to enrich for microorganisms in a biological sample, for example enrich for a desired type of bacteria within a biological sample. The microfluidic devices and methods of the present invention can be adapted to perform highly efficient lysis of microorganisms within a biological sample for diagnostic tests, for example for diagnosis of infectious agents and pathogens, such as bacteria, viruses or parasites.

### CROSS REFERENCED APPLICATIONS

This application claims benefit under 35 U.S.C. 119(e) of U.S. Provisional Patent Application Ser. No. 60/921,404 filed on Apr. 2, 2007, and U.S. Provisional Patent Application 60/925,445 filed on Apr. 20, 2007, the contents of each are incorporated herein in their entity by reference.

### FIELD OF THE INVENTION

The present invention relates generally to bacterial lysis, and more particularly to methods for bacterial lysis using a microfluidic device. The present invention relates to a device and methods for their manufacture as well as isolation, purification and detection of biological molecules, such as nucleic acids and proteins. Specifically, the invention relates to the preparation of microfluidic device comprising a polymer embedded with carbon particles and methods for cell lysis using such microfluidic device. In particular, the methods relates to the lysis of bacteria using a microfluidic device. The device can also optionally comprise modules for solid-phase isolation, purification and detection of biological molecules from the lysed cells. The device can be used, for example, for diagnostic assays and detecting microorganisms, such as bacteria, viruses and parasites.

### BACKGROUND OF THE INVENTION

Microfluidics is a multi-disciplinary field that focuses on the study of micro scale flows and their behavior. Microfluidic systems generally utilize microliter, nanoliter or even picoliter volumes of fluid and take advantage of fluid behavior at those scales. Microfluidics has emerged in recent years as a viable technology for commercialization. Application driven research and development has begun to yield useful products, some of which have been successfully commercialized.

The applications of microfluidics are varied. Some areas that have received significant attention are accurate mass flow control, high performance electronics thermal management and ink jet printing. While these applications may represent significant markets, perhaps the largest potential use for microfluidics is in biotechnology. Micro biological analysis devices, known by several different names such as MicroTAS, (Micro Total Analysis Systems) and Lab-on-a-Chip have the potential to revolutionize the healthcare industry.

While many biochemical processes can be accomplished via Lab-on-a-chip based systems one area where high growth is expected is genetics-based testing and diagnostics. According to a 2005 market report prepared by Frost and Sullivan the genetic diagnostics market is expected to grow at an average rate of 16% per year through 2011.

Molecular biological diagnostic assays in which nucleic acids are manipulated and analyzed have begun to show their value as a tool of both researchers and clinicians. By analyzing a sample to determine its genetic make-up it is believed more specific and efficient diagnostic assays can be executed as compared to protein-binding based assays. As research continues to uncover the connections between various conditions and their genetic markers the value of genetics-based diagnostics will continue to increase A Lab-on-a-Chip system for genetics based diagnostics may be comprised of several different processes traditionally carried out on separate instrumentation within a laboratory. By integrating these processes into a single platform it is hoped that one can execute a genetics based diagnostic assay more quickly and cost effectively than is currently possible.

There are many characteristics of microfluidic Lab-on-a-Chip devices which make them attractive for both research and clinical use. Their scale allows biological assays to be carried out using miniscule amounts of both the sample being tested and the reagents that are needed for processing. Combined with the cost advantages of batch production resultant of their size and manufacturing methods, Lab-on-Chip devices promise to provide a significant economic benefit versus their macro-scale counterparts. Additionally, the size of Lab-on-Chip devices frequently result in superior assay processing speed due to the shorter travel lengths, lower thermal masses and smaller fluid volumes involved. Perhaps one of the most important benefits of Lab-on-Chip devices is the potential for automation and integration that they will bring to the assays that are conducted on them. Many labor intensive biological procedures that are typically conducted in a serial fashion using many pieces of equipment may be able to be replaced with a single device containing one or more Lab-on-Chip devices. While many individual processes have been demonstrated as Lab-on-Chip devices, the potential for the integration of these micro devices as part of a larger system for specific applications has only begun to be realized.

Most current microfluidic chip-based devices are made by photolithographic patterning of silicon or glass, or with polydimethylsiloxane (PDMS) using the methods of multilayer soft-lithography. Silicon and glass fabrication can be very expensive, while PDMS lacks dimensional stability and has limited shelf-life. These limitations necessitate the use of alternative materials to make disposable, point-of-care devices, for example, for diagnostic applications. Polymer-based microfluidic chips have been described in the art, for example, U.S. Patent Application No. 2004/0101442. This application described formation of surface-modified microfluidic devices, wherein the microchannel surfaces are physically altered to increase surface area and can be chemically altered to provide additional features. However, this device is not suitable for lysis of the cells, in particular lysis of bacteria cells.

Microfluidic approaches to DNA purification have been previously demonstrated in glass microchips fabricated by Deep Reactive Ion Etching (DRIE). Recovery of DNA molecules was achieved by packing microchannels with silica particles and immobilizing by a sol-gel method. Currently used methods use nickel alloy molds made with LIGA or electroforming in hot embossing micro scale features into polymeric substrates. These methods are very expensive.

The methods used for monolith formation and attaching the solid phase to the walls of the micro channels employ heating a slurry of tetraethylortho-silicate (TEOS), ethanol and silica particles, a monolith that is covalently attached to the walls of the glass microchip is achieved. However, the sol-gel chemistry involves high temperatures and is not suitable for in situ applications of the polymeric devices.

However, for a Lab-on-Chip device to be effective in genetics-based testing and/or diagnostics, the device needs to efficiently extract the genetic material from the tissue or cell sample. The extraction and detection of biomolecules, such as nucleic acids and proteins, from cells, including eukaryotic and prokaryotic cells, is a vital step in many biological and diagnostic applications. Hence, there has been a growing interest in integrating the cell lysis and purification processes of these biomolecules on chip-based microfluidic devices. Such a device would allow higher throughput, lower sample/reagent consumption and significant cost reduction. While mammalian cells can be lysed by a combination of lysis buffer and simple mixing, lysis of bacteria cells takes significantly more effort due to the nature of the cell wall. Gram positive bacteria are particularly resistant to lysis.

In existing Lab-on-a chip devices, cells are typically lysed outside the microchip with conventional methods before the on-chip experiment, and microliters of the cell lysate or purified DNA sample is loaded on the chip for subsequent processing, for example DNA isolation and/or analysis of biomolecules. Such methods are difficult to implement in other than full diagnostic laboratory settings. This prevents them from being used for, example critical bacterial strain detection when analyzing causative agents for infections, or when the sample is in limited supply. Further, the conventional methods of cell lysis, for example bacterial lysis require many labor intensive biological procedures that are typically conducted in a serial fashion using numerous different pieces of equipment and/or solutions.

Current approaches to cell lysis on a chip presently involve a combination of chemical means, such as lysis buffers and mechanical means such as ultrasonification or mechanical agitation such as use of a ultrasonic transducer. Such lysis methods have multiple limitations, for example chemicals as a means to lyse bacteria is not desirable for several reasons: Firstly, lysis buffers and enzymes can drive device cost, and thus their use should be minimized. Second, lysozyme which is typically required for lysis of bacteria, must be mixed fresh in distilled water before each use to maximize effectiveness. This makes for either additional logistical difficulty for the device user or additional device complexity, needing to add a chemical mixing module to the overall system. Third, an incubation period, often ten to thirty minutes with the lysis buffer is typically required to ensure that the detergents and enzymes are able to fully break down cellular walls. Finally, overuse of chemicals can complicate downstream processing by interfering with extraction, polymerase chain reaction or electrophoresis.

Other methods that involve mechanical means also have their limitations, for example additional design complexity and need for additional, more complex fabrication methods than are needed for most passive devices. The addition of potentially costly transducers and electrical interconnects to an otherwise very simple design may compromise the desire to have a device that is affordable to fabricate and is single use disposable. Compounding the issue is the need for external power supplies, heater elements or ultrasonic transducers, which would be burdensome and undermine the device use as a true point-of-care diagnostic instrument.

Approaches to lyse cells on Lab-on-a-chip devices have been attempted using a variety of techniques, with each approach dependant on the organism being lysed. Chemical/Enzymatic cell lysis, mechanical lysis, thermal cycling lysis, boiling lysis, electrochemical lysis, electroporation lysis, and ultrasonic lysis have all been demonstrated in Lab-on-a-Chip devices.

Lysis can be passive lysis, requiring no assistance from electrical, mechanical or thermal transducers that are typically driven by off-chip means, or active lysis methods, requiring actuation from an external source to drive a transducer.

Current passive lysis methods used on lab-on-a-chip devices have multiple limitations. For example, chemical/enzymatic lysis by Wang et al. demonstrated a system fabricated from polycarbonate for cell lysis and DNA isolation of Bacillus Cereus, a gram-positive anerobic bacterium, in which lysis was completed by incubating the sample in detergents and lysozyme within a chamber, but was highly time and energy-dependent, requiring a minimum of an incubation period of 15-30 minutes and necessitated the use of timed heaters and valves within the chip. El-Ali et al. presented a similar approach for lysing human Jurkat E6-1 cells in a cell signaling analysis device, where the sample was held at elevated temperature and detergents affected cell lysis as the sample traversed a serpentine channel with many passes⁸. This method required the use of an extensive amount of “chip real estate”, with the lysis portion of the chip driving overall chip size. Sethu et al. took a similar flow-through approach to the lysis of red blood cells⁹. Heo et al. immobilized Escherichia Coli (E. Coli) within a microfluidic channel and flowed lysis buffers through it to affect cell lysis¹⁰. This method required suspending the E. Coli in a hydrogel precursor and UV crosslinking the hydrogel, requiring the use of a UV crosslinker for each sample. Schilling et al. utilized lateral diffusion of a bacterial protein extraction reagent into a flowing sample of E. Coli bacteria to remove M-galactosidase, a large intracellular enzyme¹¹. Finally, Bhattacharyya and Klapperich demonstrated lysis of human dermal fibroblasts by mixing them with lysis buffers and guanidinum thiocyanate and flowing them through a serpentine channel at ambient temperature¹².

In general, the extensive use of chemicals as a means to lyse bacteria is not desirable for several reasons. Firstly, lysis buffers and enzymes can drive device cost, and thus their use should be minimized. Second, lysozyme which is typically required for lysis of bacteria, must be mixed fresh in distilled water before each use to maximize effectiveness. This makes for either additional logistical difficulty for the device user or additional device complexity, needing to add a chemical mixing module to the overall system. Third, ten to thirty minutes of dwell time are typically required to ensure that the detergents and enzymes are able to fully break down cellular walls. Finally, overuse of chemicals can complicate downstream processing by interfering with extraction, polymerase chain reaction or electrophoresis.

Current mechanical forces to drive cell lysis on lab-on-a-chip devices also have multiple limitations. Lee et al. demonstrated a reagent-less lysis device in which lysis of HL-60 cells from sheep blood were lysed via a filter with micromachined nanobarbs with tip diameters less than twenty-five nanometers¹³. However, Lee et al., do not demonstrate the device was effective in lysing bacterial cells. Furthermore, the construction method utilized to make the nanobarbs in silicon is not transferable to polymer based constructions due to limitations in the replica molding process used to create features.

Current active lysis methods used on lab-on-a-chip devices also have multiple limitations. Active lysis methods include ultrasonic, thermal and electrical transducers. Ultrasonic lysis has been demonstrated by several groups. Northrop, et al. demonstrated ultrasonic lysis of Bacullus Subtilis spores trapped in a 0.22 micron filter¹⁴. The use of ultrasonic energy resulted in significant lysis; however, the ultrasonic horn used to perform the lysis was significant in size and was not integrated in the microfluidic device. Northrop et al. also demonstrate integrated selective ultrasonic lysis of epithelial and sperm cells on a chip by incorporating a sonication module to the fluidic chip design¹⁵. While effective, this approach leads to a complex design and significantly more fabrication challenges than are present in the passive lysis methods.

Thermal lysis involves the use of thermal energy affect cellular lysis. Liu et al. demonstrated thermal lysis of trapped E. Coli cells within a fully integrated biochip utilizing an embedded heater to heat a chamber that is in turn used for PCR¹⁶. Wang et al. lysed E. Coli as part of a microfluidic pathogen detection system by trapping them in a chamber of microfluidic device and using an embedded heater to boil the media in which they were suspended¹⁷. Thermal energy is also used frequently to accelerate chemical and enzymatic approaches. Electrical lysis appears quite frequently in the literature as an option for both mammalian and bacterial cells. Hellmich et al. demonstrated electrical lysis of a single Sf9 insect cell by appling an electric field pulse of 1250V/cm across a channel in which the cell resided¹⁸. Poulsen et al. demonstrated single cell lysis of a human Jurkat cell by applying a 75 Hz AC field and forcing the cell through the area where the field is present¹⁹. Lu et al. utilized an electrical field across a microfluidic channel to perform selective cellular membrane lysis, also known as electroporation, on human carcinoma cells to create permanent holes in cell plasma membranes while leaving organelle membranes intact, (electroporation is typically used to create temporary holes in cell membranes to deliver genetic material or therapeutics)²⁰. McClain et al. utilized a DC electric field of 450 to 900V/cm to lyse human Jurkat cells within a microfluidic channel for downstream use in single cell analysis²¹. Some investigators utilized both chemical and electrical means to perform electrochemical cell lysis. An example of this is research conducted by Lagally et al. where lysis of E. Coli is performed by using electrically driven dielectrophoretic forces in conjunction with the chaiotropic salt guianidinium thiocyanate²².

While the active lysis methods have shown to be effective, the main drawbacks associated with utilizing these methods are the additional design complexity and need for additional, more complex fabrication methods than are needed for most passive devices. The addition of potentially costly transducers and electrical interconnects to an otherwise very simple design may compromise the desire to have a device that is affordable to fabricate and is single use disposable. Compounding the issue is the need for external power supplies, heater elements or ultrasonic transducers, which would be burdensome and undermine the device use as a true point-of-care diagnostic instrument.

Problems also exist with conventional lysis methods or current lysis methods utilized on Lab-on-a-chip devices. They often require long assay times, energy, require difficult fluid handling techniques and use of solvents that can interfere with subsequent processing such as DNA isolation and analysis of biomolecules, and also relatively tailoring the lysis method to the microorganism being lysed, as well as numerous different reagents. These problems again prevent these assays from becoming a point-of-care diagnostic technique.

For example, for many infectious diseases, effective treatments are available. Getting the correct treatment to a patient quickly is often hindered by the time necessary to confirm a preliminary diagnosis with a laboratory test. The benefits of a speedy diagnosis are obvious, as for example, in the case of a biological attack. More immediately, the ability to differentially diagnose patients in a hospital or nursing home setting will eliminate many unnecessary measures that are often taken to prevent the putative spread of an unspecified infection. In remote or low income areas, the ability to provide laboratory test results during the course of an office visit would greatly reduce the spread of infectious disease and the number of times a patient has to visit the clinic. In all of these cases, the impact on financial and public health costs is significant.

For example, current diagnostic methods for bacterial infections typically require time and a full scale diagnostic laboratory. For some infectious diarrheas, stool cultures have limited clinical utility. Instead, infection is established by a stool bioassay for cytotoxins that cause rounding of cultured fibroblasts (cells from a cell line) or immunoassays for the stool toxins themselves. The cytotoxicity bioassay is considered the gold standard against which other cytotoxin assays are compared, given its high sensitivity (94-100%) and specificity (99%). In this bioassay, stool is diluted with a buffer, filtered to remove bacteria and solids, and then placed in a cultured monolayer of fibroblasts. Toxins produced by the organisms disrupt the cyto skeleton and, when present at levels as low as a few molecules per cell, will cause rounding. The specificity of this cytopathic effect is confirmed by preincubating a control sample with antibodies that neutralize the toxins. Cell rounding not thus blocked is referred to as “nonspecific cytotoxicity” which occurs in only −1% of samples. The bioassay is reported as “positive” or “negative;” titers are not reported as they have no utility. Drawbacks of the cytotoxicity assay are its labor-intensive nature, attendant high cost, and the 48-72 hrs it typically takes to complete.

An example of a difficult to diagnose infectious agent is Clostridium difficile (C. Difficile). Clostridium difficile infection is one of the worst antibiotic resistant nosocomial infections in the developed world and significantly contributes to the length of hospitalization for patients. The spectrum of disease caused by C. difficile infection is broad, ranging from acute watery diarrhea with abdominal pain, low grade fever, and leukocytosis to the major complications of dehydration, hypotension, toxic megacolon, septicemia perforation, and death. Typically, C. difficile-associated diarrhea occurs in elderly hospitalized patients following antibiotic treatment; it is debilitating, and prolongs hospitalization. Recently, cases of the infection have been documented in patients outside of the usual affected groups: younger people and people not in a hospital or institutional environment. This development has been a great cause of concern in the medical community, as new strains appear to cause a more severe disease. Distinguishing C. difficile from other less serious infections with similar symptoms at onset is critical to effective patient care.

Current techniques for determining infection with C. difficile require 48-72 hrs to culture. Clostridium Difficile, also known as C. Difficile or C. Diff is a gram-positive anerobic spore-forming bacterium. It is responsible for colitis and hospital acquired diarrhea that may occur following antibiotic intake, causing approximately three million cases a year². The disease is caused by the alteration of beneficial bacteria typically found in the colon by antibiotic intake. Alterations of these beneficial bacteria then lead to colonization by C. Difficile or C. Difficile spores, which may be present in the environment. (Pothoulakis, M. D. 2001. “Clostridium Difficile Infection.” Participate, Retrieved February 2006, pp. 1-3.) Difficile attacks the human body by producing two toxins, Toxin A and Toxin B, (known to be 1000 times more potent than Toxin A3). A third toxin, CDT, is also created by some strains. The toxins then line the large intestine or colon causing inflammation and diarrhea. Difficile infections are extremely prevalent within hospitals and nursing homes where patients are frequently on antibiotic therapies and in close proximity to each other. Infections can be extremely dangerous to the elderly, significantly increasing the length of hospitalization and sometimes resulting in life threatening consequences. The condition Fulminant Colitis, which is a sudden severe inflammation of the colon, occurs in roughly three percent of patients, primarily the elderly that already ailing from unrelated diseases.4 Fulminant Colitis can be fatal if not treated in a timely fashion. Transmission of C. Difficile is common in the hospital environment as it can survive in harsh environments, (spores have been found to survive up to 56 days in temperatures of 4° C. and −20° C.5), and is frequently found on commonly touched objects and on the hands of health care workers. According to one study, twenty percent of patients either arrived with or acquired the bacterium during their hospital stay (Wheeldon, Laura. 2005. “Clostridium Difficile: Return of the Old Enemy.” Microbiologist pp. 33-35; Pothoulakis, M. D. 2001. “Clostridium Difficile Infection.” Participate, Retrieved February 2006, pp. 1-3; 5 Wheeldon, Laura. 2005. “Clostridium Difficile: Return of the Old Enemy.” Microbiologist pp. 33-35; Pothoulakis, M. D. 2001. “Clostridium Difficile Infection.” Participate, Retrieved February 2006, pp. 1-3.) The standard for diagnosis of C. Difficile involves either the detection of its toxins within the stool of suspected patients or faecal culture. Faecal culture testing involves the culture of C. Difficile within a stool sample on a cycloserine cefoxitin fructose agar, (CCFA), plate. This method is known for its sensitivity but lacks in specificity in that non-toxingenic strains may produce a false positive result. This standard cytotoxicity test method is considered to be both accurate and specific for the detection of both toxins A and B. It is completed by inoculating a cell culture with filtrate of a stool sample and observing any cytopathic effects. The primary drawback for these diagnostic assays is the time involved, (twenty-four to forty-eight hours) and the need for specialized personnel to complete them. Several other enzyme immunoassays have been developed that do not require specialized personnel to complete and take less than a few hours. Companies such as Meridian Bioscience, (Premier Toxin A&B Kit, ImmunoCard Toxins A&B Kit), and Biosite, (Triage C. Difficile panel), offer kits that are claimed to produce results as quickly as 15 minutes; however, the output of these products comes in a yes/no form and gives no indication as to the level of infection. They are also known to be relatively less sensitive and less accurate than laboratory-based tests. Another problematic scenario involves the presence of smaller amounts C. Difficile, as is the case in early stage infections, that are not able to create an appreciable amount of either Toxins A or B.

Accordingly, it would be highly desirable to develop a device and a method such as a microfluidic chip which would allow application of an untreated biological sample on the chip and result in isolated and purified nucleic acids in one step. Such chips would allow cell lysis on a so-called “Lab-on-a-Chip” system, i.e. to perform lysis of cells on such a device that can be optionally coupled with another “Lab-on-a-chip” device for subsequent processing, for example but not limited to purification, detection and analysis of biomolecules from the lysed cells, for example nucleic acid or protein biomolecules. Such a Lab-on-a-chip that allows complete processing of an untreated biological sample, through to biomolecule purification and analysis on one single disposable inexpensive microfluidic chip, which would require no additional sample preparation methods, no highly skilled laboratory personnel or expensive laboratory space, and which would use a very small amount of sample and reagent material and result in rapid detection and/or isolation of one or more biological molecules in a sample. Such a “lab-on-a-chip” lysis system would greatly improve current “lab-on-a-chip” devices for the diagnosis of microorganism infections, for example bacterial infections, in a simple and quick method to greatly improve current treatment and diagnostic techniques.

### SUMMARY OF THE INVENTION

The present invention is directed to methods of manufacture of microfluidic chips such as plastic microfluidic chips, which has channels packed with a polymer embedded with carbon particles and uses thereof. The chip of the present invention is designed for application of an untreated biological sample on the chip, where the chip is capable of lysis of cells within the untreated biological sample. The chip can optionally comprise other modules enabling further processing of the biological sample, for example isolation, purification and detection of biomolecules, such as nucleic acids or proteins or peptides from the lysed cells in one step. The invention also provides a microfluidic chip comprising a cell lysis module, where the cell lysis module is capable of cell lysis of microorganisms, for example but not limited to bacteria. The microfluidic chip can further comprise additional modules for subsequently processing of the biomolecules released from the lysed cells, for example modules for isolation, purification and detection of biomolecules, thus providing a complete Lab-on-a-Chip analysis system for purifying and analysis of biomolecules from unprocessed biological samples, for example biological samples comprising cells such as bacteria and other microorganism. Examples of modules for isolation, purification and detection of biomolecules include, but are not limited to modules that isolate and analyze nucleic acids and protein biomolecules. The chips as disclosed herein can be further adapted to isolate and/or purify biomolecules, and perform highly specific immunoassays and diagnostic test, for example, for diagnosis of disease causing and/or infectious agents, such as bacteria, viruses or parasites.

For example, the microfluidic immunoassay as described herein offers significant advantages, such as, improved reaction kinetics, multistage automation potential, possibility for parallel processing of multiple analytes, and improved detection limits due to high surface area-to-volume ratio. In some embodiments, the devices as disclosed herein comprise cell lysis “lab-on-a-chip” modules and such methods of their use are portable. Accordingly, the device as disclosed herein provides an ideal point-of-care diagnostic system.

The invention is based upon a discovery that one can use a porous polymer monolith to embed particles, such as carbon particles, for example carbon nanotubes, into a polymer matrix. In the U.S. Patent Application No. 2004/0101442, Stachowiak et al. demonstrated the formation of polymer monolith inside of a cyclic olefin polymer, wherein the channel walls are modified by a polymer photografting method to encourage formation of covalent bonds with the monolith and prevent formation of voids between the channel wall and the porous monolith. However, the use of the polymer monolith for use in cell lysis or to entrap carbon nanoparticles as shown by the present invention, has not been previously shown.

Here the inventors provide a method of trapping carbon particles, for example carbon nanotubes in a porous polymer monolith to form a rapid mechanical-based cell lysis system. The monolith was formed by in-situ UV polymerization of a monomer mixture impregnated with the silica particles. The high UV transmission of for example, ZEONOR makes it suitable for in-situ photopolymerization applications. The inventors used photoinitiated polymerization prior to the formation of the monolith. The grafted interlayer polymer covalently attaches to the monolith and prevents the formation of voids between the monolith and the channel surface. The interlayer also stops the monolith from migrating down the channel during separations. The porous monolithic columns embedded with carbon particles, for example carbon nanotubes were then used for cell lysis of a variety of different biological samples comprising bacteria, including both gram positive and gram negative bacteria and subsequent analysis of extracted cell lysates using analysis of yield and quality of nucleic acid extracted from the cell lysate.

In some embodiments, the devices as disclosed herein is a sample preparation device which is useful in lysing cells in a biological sample, for example cells such as microorganisms and bacteria using an on-chip cell-lysis column. The eluted cell lysate can then be subsequently processed, for example but not limited to isolating and detecting biomolecules from the cell lysate, such as nucleic acids, antibodies, other proteins or peptides, using additional on-chip modules, for example on-chip solid-phase extraction column as previously described in U.S. Patent 2007/0015179, which is specifically incorporated herein in its entirety by reference. In some embodiments, the eluted biomolecules can be subsequently stored on-chip for downstream separation and detection tasks. The device also allows rapid and successful cell lysis. In contrast to the methods in the prior art, which require separate cell lysis before processing the biological sample on lab-on-a-chip devices, the present device allows cell lysis using a cell-lysis column, thus enabling cell lysis, isolation and purification of biomolecules from an unprocessed biological sample in a single step.

Accordingly, in one embodiment, the invention provides a microfluidic device comprising: (a) a substrate that is not glass with at least one channel of less than 150 μm in diameter, wherein the channel has an inlet, an outlet, and an internal space with a surface between the inlet and the outlet; (b) a first porous polymer monolith comprising a first monomer within the internal space, wherein the porous polymer monolith comprises a second monomer, and is attached to said first polymer in at least one region of the internal space, wherein the first and the second monomers may be of the same or different material; and (c) a second porous polymer monolith impregnated with carbon particles within said internal space.

The closed chip minimizes the possibility of contamination of the sample by the environment or contamination of the environment by the sample, both important considerations in biological sample preparation and handling. The chip also allows cell lysis, isolation and purification of, for example, nucleic acids from real-world biological samples, and their injection into a holding reservoir, wherein they can be stored for further analysis.

The microfluidic chip as disclosed herein comprises a cell-lysis module that allows cell lysis of a variety of different cells, for example mammalian cells as well as microorganisms, for example bacteria, and plant cells. Exemplary microorganisms are bacteria, for example gram-positive and gram-negative bacteria.

In some embodiments, the microfluidic chip as disclosed herein can optionally further comprise a solid-phase extraction module, for example for extraction of a variety of biomolecules, for example but not limited to many kinds of nucleic acids, including naturally occurring, synthetic and modified, DNA and RNA. The microfluidic device as disclosed herein can optionally comprise modules comprising particles that are designed to bind other biomolecules, for example particle bind to antibodies, peptides, and proteins. For example, in isolation methods from cellular material, a subsequent digestion steps can be used to obtain pure sample of only DNA or RNA. Purified nucleic acids can be easily aspirated from this reservoir. Alternatively, nucleic acid amplification, digestion, sequencing and other detection enhancing methods can be used by providing sufficient reagents, such as enzymes, buffers, primers, and nucleotides, into the reservoir. The reservoir can also be fitted into a thermocycler, for amplification and/or quantification of the nucleic acids using, for example, the PCR technique. Additionally, a detection step may be added to the system allowing detection of the biomolecules, such as cellular or bacterial antigens.

In one embodiment, the invention provides a polymer microfluidic chip with polymer-embedded carbon particles, for example carbon nanotubes, comprising a polymer matrix with at least one channel

In another embodiment, the invention provides a method of making a microfluidic chip impregnated with porous polymer comprising carbon particles, comprising the steps of providing a polymer micro-chip with at least one channel, photografting the channel by filling the channel with a pre-polymer solution or grafting mix, for example but not limited to an aromatic ketone, for example benzophenone and diacrylate solution, or a pre-polymer solution comprising methyl methacryalate (MMA) and a photo-sensitizier, for example enzophenone. In some embodiments, the pre-polymer solution can optionally comprise, for example ethylene diacrylate (EDA). The photografting step also involves irradiating the micro-chip. The photografted channel the subsequently filled with polymer solution impregnated with carbon particles, irradiating the polymer-particle mixture thereby forming a microfluidic chip impregnated with porous polymer comprising the carbon particles. In some embodiments, the channel can be washed, for example with an alcohol such as methanol, prior to filling the channel with the polymer-particle mixture comprising carbon particles.

The plastic microchips or microfluidic devices described herein for cell lysis can optionally comprise a module for sample preparation module for extraction of nucleic acids from a subject's biological samples. Extraction/purification of nucleic acids is a vital step is a number of applications, such as in methods using of nucleic acid probes for genomic DNA in the detection of human pathogens. Thus the plastic chip can function as a portable disease surveillance device, where the unprocessed biological sample can be directly applied to the plastic microchip without the need of prior cell lysis. In embodiments where the cell lysis chip further comprises a sample preparation module, the chip is can also be used for isolation of mRNA, to measure gene expression in infected cells or to determine the relative toxicity of a bacterial infection. Such a chip also provides an ideal purification system for high-speed, high-throughput DNA sequence analysis or other genomic application.

In some embodiments, the biological sample can be combined with a cationic buffer in a mixing well or mixing reservoir that is present also on the device, or in alternative embodiments a cationic buffer is added to the biological sample prior to the biological sample being passed through the cell lysis column on the cell lysis device as disclosed herein.

The proposed microfluidic cell lysis method will have advantages over the existing technologies in that a chip-based sample preparation system will shrink the conventional “bench-top” “macroscale” procedure into a miniature, portable device. The cell lysis chips of the present invention also significantly reduce the need of reagents, and also minimize sample consumption. Such chips also minimize exposure of the practitioner to possible pathogenic microorganisms in a biological sample as well as potential harmful biomolecules released from the lysed cells, for example toxins and nucleic acids released from, for example bacterial cells and other pathogenic microorganisms. The chips allow for cell lysis from small numbers of mammalian or bacterial cells and thus allow one to process many different samples in parallel.

Sample contamination can be significantly minimized by carrying out the procedures in a closed system. Since the chips are made of plastic, they will be inexpensive to produce, and thus they can be used as disposable devices. Also, the sample preparation will take place in a completely closed system, and thus greatly reduce the risk of infecting clinicians and/or the environment. Moreover, the samples can be processed and undergo cell lysis at the point-of-care for diagnostic procedures.

Accordingly, in another embodiment, the invention provides a method of lysing cells using the microfluidic chip as disclosed herein. In particular, in one embodiment, the cells are microorganisms, for example bacteria, viruses and parasites. In alternative embodiments, the cells are mammalian cells, and in further embodiments the cells are plant cells. In some embodiments, the bacteria are gram-positive bacteria, and in alternative embodiments, the bacteria are gram-negative bacteria.

In yet another embodiment, the invention provides a method of lysing cells, and subsequent purification, isolation and detection of biomolecules, for example nucleic acids using the microfluidic chip of the present invention. Such purification, isolation and detection steps is preferably performed using a microarray technology device attached after or to the collection reservoir of the cell-lysis microfluidic chip of the present invention, thus enabling isolation and purification, and potential amplification and detection of the biomolecules from the lysate immediately following cell lysis.

In one embodiment, the present invention provides a diagnostic microfluidic chip kit for lysis of cells within a biological sample, which can be optionally combined with additional microfluidic chips of having a different functions, for example but not limited to microfluidic chips comprising modules for purification of biomolecules, and/or modules for detection of biomolecules and/or modules for analysis of biomolecules, wherein the biomolecules are released from the cells lysed by the cell lysis microfluidic chip of the present invention. The kit may be tailored to the particular need, for example for a particular diagnostic use, for example but without limitation, detection of nucleic acids and/or proteins of a particular pathogen, detection of level of infection by a particular pathogen etc. The kit may be reusable or disposable. A one time disposable diagnostic chip kit is also encompassed in the present invention.

The present invention further provides a cell lysis technique for lysis of cells present in a biological sample. The microfluidic format makes the procedure rapid and highly effective at cell lysis of a variety of cells, for example microorganisms, bacteria, plant cells and mammalian cells.

In one embodiment, one uses cyclic polyolefins as chip material. This material makes the device ideal for disposable point-of-case diagnostics.

The methods of the cell lysis of the present invention also allows for cell lysis of a variety of cells from a biological sample, in particular biological samples that have low concentration of cells and/or a variety of different cell types within the biological sample. For example, a biological sample may comprise a variety of different cells, for example samples may comprise a combination of different mammalian cells, microorganisms, and different types of bacteria, each having very distinct characteristics. For example, the biological sample may comprise both gram-positive and gram-negative bacteria, and bacteria being rod-shaped, cylindrical or spiral, or any bacteria shape, and a variety of different sizes, ranging from 0.1-2.0 μm in diameter and approximately 1-10 μm in length. In some embodiments, the device as disclosed herein can be used to lyse bacteria with diameters ranging from 0.1-0.5 μm, 1.0-1.2 μm, 0.5-2.0 μm, and in some embodiments, the device as disclosed herein can be used to lyse bacteria with lengths ranging from 1.0-5.0 μm, 3.0-5.0 μm or larger. A typical range of the dimensions and types of bacterial with respect to main characteristics such as (i) gram-negative or gram-positive, (ii) diameter, (iii) length, (iv) anaerobic properties (v) spore forming properties, and (iv) chain forming properties are outline in Table 3.

In some embodiments, the biological sample is obtained from a subject via non-invasive means, for example, a saliva sample, urine or stool sample. In alternative embodiments, the biological sample is a biopsy or other tissue sample. However, traditional methods prevent cell lysis of a variety of different cells in a biological sample because the lysis method is dependent on the cells requiring to be lysed. The mechanical based cell lysis method using the device as disclosed herein makes the system ideal for cell lysis of a variety of different cell types simultaneously, particularly wherein different types of cells are present within a single biological sample.

Accordingly, the present invention also provides a method for cell lysis, in particular bacterial cell lysis from biological samples. In one embodiment, the present invention provides a device and method for cell lysis, for example bacterial cell lysis of disease-causing and/or pathogenic bacteria in a point-of-care system. For example, diagnostic chip and methods for cell lysis of diarrhea caused by Clostridium difficile (C. difficile) are provided.

One aspect of the present invention relates to a microfluidic device comprising; (i) a substrate with at least one channel; wherein the channel has an inlet, an outlet and an internal space with a surface between the inlet and the outlet; and (ii) a porous monolith within the internal space of the channel, wherein the porous monolith is embedded with a plurality carbon nanotubes. In some embodiments, the channel can be of any geometrical pattern, for example it can be a straight line or a curve or in some embodiments a serpentine-shaped channel between the inlet and the outlet.

In some embodiments, the carbon particles embedded in the polymer monolith can be any carbon particle known by persons of ordinary skill in the art, such as but not limited to carbon nanotubes, such as single walled nanotubes (SWNT) or multiple (or multi-) walled nanotubes (MWNT). In some embodiments, the carbon nanotubes useful in the microfluidic device as disclosed herein can be between about 1-20 microns long, or between about 5-15 microns long, or longer than 20 microns. In some embodiments, the carbon nanotubes useful in the microfluidic device as disclosed herein can be of any diameter, for example less than 100 microns in diameter, or less than 90 μm in diameter or alternatively, greater than 100 microns in diameter. In some embodiments, the carbon nanotubes can be within the range of about 90-10 μm in diameter, for example, they can be about 90, or about 80, or about 70, or about 60, or about 50, or about 40, or about 30, or about 20, or about 10 microns in diameter.

Another aspect of the present invention relates to the use of the microfluidic device as disclosed herein in combination with a solid-phase extraction column, wherein the inlet of the solid-phase extraction column is connected to the outlet of the channel comprising the monolith embedded with carbon particles, and wherein a sample can be passed through channel comprising the carbon embedded monolith to the solid-phase extraction (SPE) column. Accordingly, in such an embodiment, the lysate from the biological sample which has been processed by the cell lysis microfluidic device as disclosed herein can be subsequently processed, for example, for extraction, purification and/or isolation of biomolecules released from the lysed cells. In some embodiments, the cell lysis microfluidic device as disclosed herein can be combined with a module enabling PCR of the biomolecules released from lysis of the cells using cell lysis device as disclosed herein, thus providing a complete lab-on-a-chip system for analysis of biomolecules from cells, such as bacteria, such as gram positive and gram negative bacteria. In some embodiments, the SPE comprises a silica bead and polymer composite as disclosed in U.S. Patent application 2007/0015179 which is incorporated herein by reference.

Another aspect of the present invention relates to the use of the microfluidic device as disclosed herein which further comprises a filter membrane, wherein a outlet of the filter membrane is connected to the inlet of the inlet of the channel comprising the monolith embedded with carbon particles, and wherein a sample can be passed through the filter membrane prior to the channel comprising the carbon embedded monolith. In such an embodiment, the microfluidic device can be used to enrich for microorganisms such as bacteria within a biological sample. In some embodiments, the elutant which has been through the filter membrane is passed through the channel comprising the carbon embedded monolith, which is useful for example for cell lysis of microorganisms which have not been filtered by the filter. Such an embodiment is useful to select out (i.e. exclude) specific microorganisms, such as large bacteria, and only pass through the cell lysis microfluidic device of the present invention bacteria within the biological sample that are of a smaller size to pass through the filter.

In an alternative embodiment, the microorganisms which have been collected on the filter membrane can be harvested and subsequently passed through the channel comprising the carbon embedded monolith of the microfluidic device as disclosed herein. Such an embodiment is useful to specifically select (i.e. include) specific microorganisms, such as microorganisms of a specific size or diameter, such as large bacteria to be pass through the cell lysis microfluidic device of the present invention, with the microorganisms such as bacteria which are in the biological sample that are of a smaller size to passing through the filter, and this not subjected to subsequent cell lysis using the microfluidic lysis device as disclosed herein.

In some embodiments, the microfluidic device as disclosed herein comprises a substrate which is glass or a variant thereof, and in some embodiments, the substrate is not glass, for example it can be selected from the group such as plastic, metal, silica or some other material which is known by persons of ordinary skill in the art as a substrate for microfluidic devices.

Another aspect of the present invention relates to a method for bacterial lysis, the method comprising: (i) suspending the bacteria in a suspension buffer; (ii) passing the bacteria through a plurality of carbon nanotubes; wherein the plurality of carbon nanotubes contact the bacteria and lyse the bacteria.

Another aspect of the present invention relates to a method for bacterial lysis and DNA extraction in a single step, the method comprising: (i) suspending the bacteria in a suspension buffer, (ii) passing the bacteria through a plurality of carbon nanotubes; and (iii) passing the bacteria from step (ii) through a solid-phase extraction (SPE) column, wherein the plurality of carbon nanotubes and the solid-phase extraction column are located on a solid support. In some embodiments, the suspension buffer is a chaotropic buffer, and in some embodiments the suspension buffer is B1 as disclosed herein in the Examples. In some embodiments, the suspension buffer further comprises at least one detergent. In some embodiments, the bacteria for example bacteria in a biological sample are passed through the carbon nanotubes under pressure.

In some embodiments, the devices and methods as disclosed herein comprises a plurality of carbon nanotubes embedded in a monolith, for example a polymer monolith embedded with carbon nanotubes. In some embodiments, the carbon particles embedded in the polymer monolith can be any carbon particle known by persons of ordinary skill in the art, such as but not limited to carbon nanotubes, such as single walled nanotubes (SWNT) or multiple (or multi-) walled nanotubes (MWNT). In some embodiments, the carbon nanotubes useful in the microfluidic device as disclosed herein can be between about 1-20 microns long, or between about 5-15 microns long, or longer than 20 microns. In some embodiments, the carbon nanotubes useful in the microfluidic device as disclosed herein can be of any diameter, for example less than 100 microns in diameter, or less than 90 μm in diameter or alternatively, greater than 100 microns in diameter. In some embodiments, the carbon nanotubes can be within the range of about 90-10 μm in diameter, for example, they can be about 90, or about 80, or about 70, or about 60, or about 50, or about 40, or about 30, or about 20, or about 10 microns in diameter.

In some embodiments, the methods comprises passing the lysate from the cell lysis microfluidic device as disclosed herein through a SPE column. In some embodiments, the SPE comprises a silica bead and polymer composite as disclosed in U.S. Patent application 2007/0015179 which is incorporated herein by reference.

In some embodiments, the methods and microfluidic devices as disclosed herein comprise a solid support, wherein the solid support can be of any material or substrate which is known by persons of ordinary skill in the art suitable for such a microfluidic device In some embodiment, the solid support is a chip, which comprises, for example glass or a variant thereof, and in some embodiments, the solid support is not glass, for example it can be selected from the group such as plastic, metal, silica or some other material which is known by persons of ordinary skill in the art as a substrate for microfluidic devices.

In some embodiments, the methods and microfluidic devices as disclosed herein are useful for the lysis of microorganisms, such as, for example bacteria, such as gram-negative bacteria. Any gram negative bacteria can be lysed by the methods and devices as disclosed herein, including but not limited to E. Coli. In some embodiments, gram-positive bacteria can be lysed by the methods and devices as disclosed herein, for example but not limited to B. subtillis or C. Difficile.

Another aspect of the present invention relates to a method for obtaining nucleic acids from a cell using the methods and microfluidic devices as disclosed herein. In some embodiments, the nucleic acid is any nucleic acid, for example but not limited to, DNA or RNA, including miRNAs, mRNA, tRNA and the like. In some embodiments, nucleic acids are obtained using the methods and devices as disclosed herein from bacterial such as gram negative and/or gram-positive bacteria. Nucleic acid can be obtained from any gram negative bacteria using the methods and devices as disclosed herein, including but not limited to E. Coli. In some embodiments, nucleic acid can be obtained from any gram-positive bacteria using the methods and devices as disclosed herein, for example but not limited to B. subtillis or C. Difficile.

In some embodiments, the sample is passed though the microfluidic devices as disclosed herein under pressure, for example but not limited to pressure applied by way of a syringe or other equipment useful to generate a pressure system.

In some embodiments a cell is suspended in a suspension buffer prior to use in the methods and devices as disclosed herein, for example the cell can be suspended in a lysis buffer such as, for example a chaotropic buffer. In some embodiments, the lysis buffer further comprises at least one detergent.

Another aspect of the present invention relates to the methods and use of the microfluidic device as disclosed herein for the lysis of cells, and in some embodiments, for methods to obtain biomolecule such as nucleic acid (such as DNA or RNA) from such cells. In some embodiments, the cell is a microorganism, such as, but not limited to bacteria, including gram-negative bacteria (such as E. Coli) and gram-positive bacteria (such as B. subtillis or C. Difficile).

### BRIEF DESCRIPTION OF FIGURES

FIG. 1 shows a schematic rendering of a Polymer Lab-on-a-Chip System for Diagnostics. As shown in FIG. 1, in one embodiment the present invention provides a Lab-on-a-Chip system which comprises a cell lysis module, and optionally a solid phase extraction and isolation module, and optionally a PCR amplification module. As shown in FIG. 1, in some embodiments sample is introduced into one of the inlet wells shown and a lysis buffer is introduced into the other inlet well and the two flows mix through the cell lysis module. The lysis module lyses the cells and outputs nucleic acids suspended in a complex mixture. The filtration module filters the complex mixture, leaving only small molecules such as proteins and nucleic acids to move on to the extraction and isolation module. The extraction and isolation module extract the nucleic acids from the complex mixture and the remainder of the sample goes into the waste well. A wash buffer is introduced into the system to wash any nucleic acids stuck upstream of the extraction and isolation module and to clean out the remainder of non-nucleic acid material stuck upstream of the waste well. The nucleic acids are then resuspended in an elution buffer injected from another inlet and enter the polymerase chain reaction module. The polymerase chain reaction module repeatedly heats and cools the nucleic acids in the presence of the enzyme polymerase and denatures the dsDNA into single stranded DNA, (ssDNA). Additional complimentary based pairs are added via an inlet well and join with the denatured ssDNA to recreate dsDNA. This results in the exponential replication of dsDNA. The sample arrives at the device outlet well as a pure, high concentration suspension of dsDNA suitable for analysis via electrophoresis.

FIG. 2 shows a solid model of Microfluidic Lysis Device. As shown in FIG. 2, the microfluidic device consists of three basic parts, the base, including channels and feedthrus, the cover and the ports. The device was fabricated by thermally pressing Zeonex 690R (which comes in pellet form), to create a base and cover disk and then using Ni-plated steel wires ˜430 um in diameter to and 2 cm long to thermally emboss microchannels within the substrate. A hand drill was then used to drill from the embossed side of the chip at each end of the channel to create a feed-thru. The cover disc is then thermally bonded to the base disk by the hot press controlled to roughly 276-278° F. (just higher than the glass transition temperature of the material) to the base disk enclosing the microfluidic channels and creating a fluid seal. Several repeated bonding steps were used in order to reach a total seal of the cover plate with the base plate, and depending on the differing numbers of bonding steps for each chip, the channel diameter tended to vary anywhere from 100-400 um after bonding, and sometimes varying in diameter along the length. Nanoport assemblies, (P/N N-333), manufactured by Upchurch Scientific of Oak Harbor, Wash. were then epoxied to the chip with JB Weld Epoxy creating a threaded connection to the feed-thrus. Usually attachment of the Nanoport assemblies followed in-situ polymerization of the porous polymer monolith.

FIG. 3 shows a depiction of a Single-Walled Nanotube (SWNT).

FIG. 4 shows a transmission Electron Micrograph, (TEM) of multi-walled nanotubes (MWNT), from NanoLabs (lot PD15L15-00405 MWNT).

FIG. 5 shows an example of a scanning Electron Micrograph image of a Butyl Methacrylate (BUMA) based porous polymer monolith.

FIG. 6 shows an example of a an optical Microscope Inage of a Microfluidic Channel containing a BUMA porous Polymer Monolith

FIG. 7 shows an example of a Scanning Electron micrograph (SEM) Image of Microfluidic Channel containing a BUMA Porous Polymer Monolith.

FIG. 8 shows an example of a SEM Image of a Carbon Nanotube Clump in a BUMA Porous Polymer Monolith.

FIG. 9 shows an example of a SEM of Carbon Nanotube “Barbed Wire” Structure within a BUMA Porous Polymer Monolith, demonstrating the disorganized and random orientation of the carbon nanotubes on the surface of the polymer monolith.

FIG. 10 shows an example of a SEM Image of Microfluidic Channel containing a GMA Porous Polymer Monolith.

FIG. 11 shows an example of an optical Microscope image of GMA based porous polymer monolith.

FIG. 12 shows an example of a SEM Image of Suspected Polymer Wrapped Carbon Nanotubes imbedded in a GMA Porous Polymer Monolith.

FIG. 13 shows an example of an optical Microscope Images of “Nanotube Wall” Device Design.

FIG. 14 shows an example of a SEM of Carbon Nanotubes within a BUMA Porous Polymer Monolith Fabricated with Polar Solvents.

FIG. 15 shows an example of a sample Full Range Quant-it Picogreen Standard Curve.

FIG. 16 shows an example of an experimental Apparatus for Testing Microfluidic Devices.

FIG. 17 shows an example of the results from BUMA Device Testing with E. Coli Suspended in 0.85% NaCl. dsDNA Extracted from E. coli was run through BUMA/CNT (Non-Polar) PPM microfluidic Device. The sample cell concentration was ˜1-3×10⁹ cells/ml. 0.85% NaCl Media was used and fluorescence was detected at 480 nm and emitted at 520 nm.

FIG. 18 shows an example of the results of BUMA Device Testing at Varying Sample Flowrates. dsDNA Extracted from E. coli was run through BUMA/CNT (Non-Polar) PPM microfluidic Device at various flow rates. The sample cell concentration was ˜3−9×10⁹ cells/ml. 0.85% NaCl Media was used and the fluorescence was detected at 480 nm and emitted at 520 nm.

FIG. 19 shows an example of the results from BUMA device testing with Buffer B1 suspended E. Coli. dsDNA Extracted from E. coli was run through BUMA/CNT (Non-Polar) PPM microfluidic Device. The sample cell concentration was ˜5×10⁷ to 1×10⁸ cells/ml. Buffer B1 was used and fluorescence was detected at 488 nm and emitted at 525 nm.

FIG. 20 shows an example of the results from GMA Device Testing with E. Coli Suspended in 0.85% NaCl. dsDNA Extracted from E. coli was run through GMA/CNT (Polar) PPM microfluidic Device. The sample cell concentration was about 2×10⁸ to 1×10⁹ cells/ml. 0.85% NaCl Media was used and the flow rate was 300 μl/hr. Fluorescence was detected at 488 nm (excite) and emitted at 525 nm.

FIG. 21 shows an example of the results from GMA device testing with Buffer B1 suspended E. Coli. dsDNA Extracted from E. coli was run through GMA/CNT (Polar) PPM microfluidic Device. The sample cell concentration was ˜6×10⁸ to 1×10⁹ cells/ml. Buffer B1 was used and the flow rate was 500 μl/hr. Fluorescence was detected at 488 nm (excite) and emitted at 525 nm.

FIG. 22 shows an example of the results from GMA device testing with B. Subtilis suspended in 0.85% NaCl. dsDNA Extracted from B. Subtillis with GMA/CNT (Polar) PPM microfluidic Device. The sample cell concentration was about 1×10⁹ cells/ml. 0.85% NaCl Media was used and the flow rate was 500 μl/hr. Fluorescence was detected at 488 nm (excite) and emitted at 525 nm.

FIG. 23 shows an example of the results from GMA device testing with B. Subtilis suspended in buffer B1. dsDNA Extracted from B. Subtillis with GMA/CNT (Polar) PPM microfluidic Device. The sample cell concentration was about 1×10⁹ cells/ml. Buffer B1 was used and the flow rate was 500 μl/hr. Fluorescence was detected at 488 nm (excite) and emitted at 525 nm.

FIG. 24 shows the generation of the monolith embedded with carbon nanotubules (CTNs). FIG. 24A shows a schematic representation of cross section through a channel, showing steps for generating of a channel comprising the carbon nanotubule embedded monolith FIG. 24B shows a schematic representation of the a solid support comprising a channel (back line) which comprises the monolith embedded with carbon nanotubules (CTNs), where a sample to be lysed, for example a sample comprising cells or bacteria is passed along the channel comprising the monolith embedded with carbon nanotubules (CTNs). The sample, for example a sample comprising bacteria, is passed along such a channel under pressure, for example using a syringe, and the lysed bacteria is collected at the opposite end of the channel to which it was inserted. The lysed bacteria can then undergo further processing, for example nucleic acid extraction according to the methods of the present invention. FIG. 24C shows a scanning electronic microscope (SEM) photograph of the monolith embedded with carbon nanotubules (CTNs).

FIG. 25 shows bacterial dsDNA quantified with PicoGreen (488/525 nm) after suspension in 0.85% NaCl+4% Proteinase K and run through CNT lysis column. The bacterial samples (100 μl) were run through the lysis column at 450 μl/hr, filtered with a 0.2 μm filter, precipitated with ethanol, and quantified with PicoGreen at an excitation of 488 nm and emission of 525 nm. The positive control was lysed and the DNA was isolated using a Qiagen kit.

FIG. 26 shows an example of a comparison of bacterial DNA isolated using Qiagen lysis kit and isolated using the Qiagen kit, with the Delta Rn vs. Cycle # shown. Experimental samples were lysed on-chip and extracted using the SPE column.

FIG. 27 shows an example of a SEM Image of a Carbon Nanotube Clump in a BUMA Porous Polymer Monolith, where the polymer is of the non-polar BUMA/CNT formulation.

FIG. 28 shows an example of a SEM Image of Suspected Polymer Wrapped Carbon Nanotubes imbedded in a GMA Porous Polymer Monolith.

FIG. 29 shows an example of a SEM of Carbon Nanotubes within a BUMA Porous Polymer Monolith Fabricated with Polar Solvents.

FIG. 30 shows a bacterial dsDNA quantified with PicoGreen (488/525 nm) after suspension in 0.85% NaCl+0.8 mg/mL Proteinase K and run through microfluidic lysis column. The bacterial samples (100 μl at a concentration of 10⁵ colony forming units (CFUs) per milliliter) were run through the lysis column at 450 μl/hr, filtered with a 0.22 μm filter (to remove intact bacteria and bacteria cell walls), precipitated with ethanol, and quantified with PicoGreen at an excitation of 488 nm and emission of 525 nm. The positive control was lysed and the DNA was isolated using a Qiagen kit. The negative control was resuspended in the same NaCl suspension and was not run through a lysis column, instead it was only filtered and ethanol precipitated. The microfluidic lysis columns perform to with the same efficiency as compared with the Qiagen kit for both gram-positive and gram-negative test species.

FIG. 31 shows RT-PCR amplification threshold (C_{T}) values for GFP transfected E. coli with GFP primers. A higher C_{T} value means less recovery. The plot is data from integrated 4 cm channels with the lysis column streamlined (in line) with the extraction column. The experimental samples (˜10⁵ CFU/ml) were resuspended in chaotropic buffer with 0.8 mg/mL of proteinase K with 0.01% SDS, lysed with the lysis column and extracted using the SPE column. The positive controls were lysed and extracted using the Qiagen kit. The negative control shown here is the bacteria sample suspended in the same chaotropic buffer, filtered with a 0.22 μm filter and ethanol precipitated. The PCR no template control did not amplify. The combined columns perform with the same efficiency as compared with the Qiagen kit.

FIG. 32 shows a Simulated Sepsis Amplification Threshold (C_{T}) Values for GFP transfected E. coli with GFP primers at two concentrations, 10³ and 10² CFU/mL resuspended in human whole blood. The positive controls were lysed and extracted using the Qiagen lysis kit. The negative controls did not amplify. This demonstrates that the device of the present invention was better than the Qiagen kit for purifying and extracting DNA at concentrations of bacteria between the range of 10³ and 10² CFU/mL.

FIG. 33 shows a comparison of the relative recovery of E. coli DNA from human urine samples between the micro extraction columns to the positive control (Qiagen kit) for bacteria concentrations 1×10⁴−1×10¹ CFU/mL. As demonstrated here, a higher ratio indicates better recovery of DNA. The device of the present invention also outperforms and is better than the Qiagen kit for recovery of bacterial DNA from human samples in the range of 10⁴-10¹ CFU/mL. The negative controls (no template) did not amplify during the experiment.

FIG. 34 shows schematics of production of some embodiments of the device described herein. FIG. 24A shows a schematic diagram of hot embossing of the chips after microfabrication of the mold, and FIG. 24B shows a schematic diagram of the micro total analysis chip showing sample introduction, filtration, lysis, and nucleic acid separation and PCR amplification steps. The box shaded strip is of the right of the coil represents the heating element. FIG. 24C shows computational simulation results for the temperature distribution in a single-heater on-chip PCR reaction chamber. The plot shows the temperatures at the top and bottom of each turn in the serpentine channel indicating that sufficient temperature difference is achieved for thermal cycling. The photograph is the channel module as built in the lab, and the three-dimensional model shows the temperature gradients through the chip indicating good temperature control. The simulations were carried out using materials properties of the plastic microfluidic chips and a heater temp of 103° C. and a flow rate of 2 microliters/min.

FIG. 35 shows an example of the microfluidic channel for the use as a filter for bacterial enrichment instead of, or prior to bacteria lysis. This picture shows bacteria collected in front of the monolith filter, with the bacteria on the left, which can then be recovered and collected from the filter to enrich the concentration of the bacteria in a sample.

FIG. 36 shows microscale filters in microfluidic channels for filtering the bacteria and bacterial enrichment. FIG. 36 shows that increased concentrations of the bacteria can be achieved from 1×10⁴ CFU/ml to 1×10⁷ CFU/ml concentrations.

### DETAILED DESCRIPTION

### General

The present invention relates generally to lysis of cells, in particular bacterial lysis, and more particularly to methods for bacterial lysis using a microfluidic device. In some embodiments, the cell lysis microfluidic device further comprises other modules for processing and analysis of biomolecules present in the cell lysate, for example the extraction of bacterial DNA using a single device. The inventors have developed a microfluidic platform for rapid on-chip lysis of bacteria for point-of-care diagnostics. The ability to diagnose bacterial infections simply and quickly will greatly improve current treatment and diagnostic techniques. The present invention provides a device which functions as a microfluidic system which is useful for disposable diagnostic applications obviating the need for full laboratories to diagnosis infections.

The present invention relates to a method to lyse cells from a biological sample, for example bacterial cells in a biological sample. The present invention provides methods to lyse cells by passing them through a column or channel comprising carbon particles, for example carbon nanotubes. In some embodiments, the column or channel comprising carbon particles, for example carbon nanotubes is a channel comprises carbon particles embedded on a polymer or monolith.

In some embodiments, the present invention provides methods to lyse cells and obtain nucleic acid, for example DNA or RNA, from the bacteria in a single step. In such embodiments the method comprises lysing the bacteria using carbon nanotubes followed by passing the sample through a solid-phase extraction column. In some embodiments, the carbon nanotubes and the solid-phase extraction column are present on a solid support.

In another aspect, the present invention relates to a device to lyse cells, and in some embodiments, the present invention relates to a device to lyse cells and obtain nucleic acids from such cells.

In some embodiments, the cells are microorganisms, for example bacterial cells. In some embodiments, the bacterial cells are gram-negative cells and in alternative embodiments, the bacterial cells are gram-positive cells. In alternative embodiments, the cells can be any cell, for example mammalian cells, plant cells and chimeric cells. In some embodiments, the cells can be from any organism or multi-cell organism. In some embodiments, the cell is a microorganism. In some embodiments, the microorganism is from any genus. In some embodiments, the microorganism is a pathogenic microorganism.

In some embodiments, the carbon particles, for example nanotubes are embedded in a monolith. In some embodiments the monolith is a polymer monolith. In some embodiments, the carbon nanotubes are between 1-20 microns in length, and in some embodiments, the carbon nanotubes are between 5-15 microns in length, however, carbon nanotubes of longer than 20 microns are encompassed for use in the present invention. In some embodiments, the carbon nanotubes are less than 100 microns in diameter, and in some embodiments, the carbon nanotubes are greater than 100 microns in diameter. Carbon nanotubes of about 90, or about 80, or about 70, or about 60, or about 50, or about 40, or about 30, or about 20 and or about 10 microns in diameter are encompassed for use in the present invention. In some embodiments, the carbon nanotubes are single walled nanotubes (SWNT) and in some embodiments, the carbon nanotubes are multi-walled nanotubes (MWNT). In some embodiments, where MWNT are used, the carbon nanotubes have about 8-10 walls, and the walls are about 3 nm in thickness.

In some embodiments, the present invention encompasses use of a solid-phase extraction (SPE) column to isolate the nucleic acids from the lysed cell, for example the lysed bacterial cell. In some embodiments, the solid-phase extraction column comprises a silica bead and polymer composite. In alternative embodiments, any solid-phase extraction column is useful in the methods of the present intervention, and such solid-phase extraction columns and nucleic acid extraction methods are commonly known by persons of ordinary skill in the art and are encompassed for use in the present invention. For example but not limited to, the following examples are useful for nucleic acid extraction according to the methods of the present invention; silica bead packed solid phase extraction column, silica membranes, high surface area pillar chip modules, Leukosorb filters and Nano-gap channel arrays.

Several methods have been analyzed at the bench scale to break apart bacteria, however, the inventors have discovered that a combination of mechanical and chemical methods is the most efficient, especially when complex patient samples (like feces) are used. The inventors have invented a device that that uses mechanical shear induced by flow disruption in addition to mixing with a buffer to break apart gram positive bacteria. In some embodiments, the present invention relates to a device comprising a lysis column that contains the polymeric solid phase impregnated with a slurry of carbon particles, for example carbon nanotubes. In some embodiments, the carbon nanotubes are about 5 to 15 microns long and less than 100 nm in diameter. The tubes are very stiff and mechanically tear open the bacterial cell walls in the presence of a chemical lysis solution.

In traditional laboratory protocols cell lysis is usually accomplished by enzymatic/chemical means, sometimes assisted by mechanical, electrical or thermal transducers. The most common approach utilizes a combination of detergents, also known as lysis buffers, frequently in conjunction with enzymes, to weaken and rupture walls or membranes of target organisms. Ultrasonication, or mechanical agitation via an ultrasonic transducer, is frequently used in addition to chemicals to exert force on cell walls and assist in lysis of plant cells and bacteria. Bead milling is a method of lysing particularly hearty organisms by placing them in a suspension with metallic, glass or polymeric beads and then agitating them to mechanically disrupt the cells. Lysis on a chip has been accomplished via a variety of techniques, however each method is dependant on the organism being lysed. Chemical/Enzymatic cell lysis, mechanical lysis, thermal cycling lysis, boiling lysis, electrochemical lysis, electroporation lysis, and ultrasonic lysis have all been demonstrated in Lab-on-a-Chip devices. In general, the extensive use of chemicals as a means to lyse bacteria is not desirable for several reasons. Firstly, lysis buffers and enzymes can drive device cost, and thus their use should be minimized. Second, lysozyme which is typically required for lysis of bacteria, must be mixed fresh in distilled water before each use to maximize effectiveness. This makes for either additional logistical difficulty for the device user or additional device complexity, needing to add a chemical mixing module to the overall system. Third, ten to thirty minutes of dwell time are typically required to ensure that the detergents and enzymes are able to fully break down cell walls. Finally, overuse of chemicals can complicate downstream processing by interfering with extraction, polymerase chain reaction or electrophoresis.

The methods of the present invention does not require an external power source. In some embodiments, the bacteria can be passed through the device using pressure applied via a syringe or other pressure generating device. In some embodiments, the device of the present invention does not require the addition of lysis chemicals or enzymes, which cuts back on the cost of lyzing the bacteria. In some embodiments, the present invention allows the composite monoliths to be prepared inside of plastic microfluidic chips without the use of frits to keep it in place. In some embodiments, the monolith is covalently linked to the inside of the channel by surface grafting techniques.

### DEFINITIONS

The term “carbon particle” a particle as used herein is intended to encompass all carbon particles, as well as structures comprising carbon and other molecules. In some embodiments, the carbon particle is comprised of carbon atoms or molecules several atoms thick for example a carbon nanotube, and in alternative embodiments, the carbon particle may be one atom or more thick, for example graphene.

The term “Lab-on-a-chip” as used herein refers to a platform to perform laboratory reactions and processes on a single microfluidic chip on a micro-scale level. Typically, lab-on-a-chip are inexpensive disposable chips that do not require highly skilled personnel or expensive laboratory space, and which allows processing of a small amount of sample material. In some embodiments, the lab-on-a-chip enable processing of a sample sequentially through multiple reactions and/or processes using a single device. Lab-on-a-chip devices are typically designed to perform a particular laboratory reaction, for example extraction and isolation of biomolecules from a biological sample.

The term “microorganism” as used herein includes ay microscopic organism or taxonomically related organisms within the categories of bacteria, algae, fungi, yeast, protozoa and the like. The microorganisms targeted can be pathogenic microorganisms.

The term “bacteria” as used herein is intended to encompass all variants of bacteria, for example, prokaryotic organisms and cyanobacteria. Bacteria are small (typical linear dimensions of around 1 m), non-compartmentalized, with circular DNA and ribosomes of 70 S. The term bacteria also includes bacteria subdivisions of Eubacteria and Archaebacteria. Eubacteria can be further subdivided on the basis of their staining using Gram stain, and both gram-positive and gram-negative eubacteria, which depends upon a difference in cell wall structure are also included, as well as classified based on gross morphology alone (into cocci, bacilli, etc.).

The term “pathogen” as used herein refers to any disease producing microorganism.

The term “pathology” as used herein, refers to symptoms, for example, structural and functional changes in a cell, tissue, or organs, which contribute to a disease or disorder.

For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the term “polymer” refers to a macromolecule made of repeating (monomer) units or protomers. The term “polymer monolith” as used herein refers to a structure, such as a, for example a column, made from the polymer.

The term “polar” as used herein, refers to a molecule that has a permanent electric dipole.

The term “microfluidics” or “microfluidics” as used here refers to the manipulation of microliter and nanoliter volumes of fluids and the design of systems in which such small volumes of fluids will be used.

The term “biomolecule” is any molecule, by itself, or in a complex with other molecules which is obtained from a cell. The term biomolecule also encompasses heterologous molecules and recombinant molecules obtained from a cell.

The term “nucleic acid” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term nucleic acid is used interchangeably with gene, cDNA, and mRNA encoded by a gene. The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyedenosine, deoxycytidine, deoxyguanosine, and deoxythymidine. For purposes of clarity, when referring herein to a nucleotide of a nucleic acid, which can be DNA or RNA, the terms “adenosine”, “cytosine”, “guanosine”, and thymidine” are used. It is understood that if the nucleic acid is RNA, a nucleotide having a uracil base is uridine. The term “nucleotide” or nucleic acid as used herein is intended to refer to ribonucleotides, deoxyribonucleotides, acylic derivatives of nucleotides, and functional equivalents thereof, of any phosphorylation state. Functional equivalents of nucleotides are those that act as substrates for a polymerase as, for example, in an amplification method and artificial types of nucleic acids such as peptide nucleic acid (PNA) and locked nucleic acid (LNA) can be used. Functional equivalents of nucleotides are also those that can be formed into a polynucleotide that retains the ability to hybridize in a sequence specific manner to a target polynucleotide. As used herein, the term “polynucleotide” includes nucleotides of any number. A polynucleotide includes a nucleic acid molecule of any number of nucleotides including single-stranded RNA, DNA or complements thereof, double-stranded DNA or RNA, and the like.

The terms “protein”, “polypeptide” and “peptide” are used interchangeably herein refer to a gene product.

The term “gene product(s)” as used herein refers to include RNA transcribed from a gene, or a polypeptide encoded by a gene or translated from RNA.

The term “Solid-phase extraction” or “SPE” is a separation method that uses a solid phase and a liquid phase to isolate one, or one type, of analyte from a solution. SPE is based on the preferential affinity of desired or undesired solutes for the solid material. In some cases, SPE is usually used to “clean up” a sample before using a chromatographic or other analytical method to quantitate the amount of analyte(s) in the sample.

The term “embedded” refers to one object contained within another object, for example a larger object comprising smaller objects or particles.

The term “lysis” as used herein refers to the rupturing of a cell membranes or cell wall and release of the cytoplasm from the cell. As used herein, the term “lysate” refers to the material produced by the destructive process of lysis.

As used herein, a “device” refers to a tool or piece of equipment which typically is used for a particular function, mechanical task or use, for example, in some embodiments of the present invention, the device is used as a tool for cell lysis.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The term “isolated” as used herein refers to the state of being substantially free of other material which is not the intended material. Stated another way, if the intended isolated product is a nucleic acid, the isolated nucleic acid is substantially free of other materials and/or contaminants such as proteins, lipids, carbohydrates, or other materials such as cellular debris or growth media. Typically, the term “isolated” is not intended to refer to a complete absence of these materials. Neither is the term “isolated” intended to refer the material is free from water, buffers, or salts, unless they are present in amounts that substantially interfere with the methods of the present invention. The term “isolated” as used herein when used with respect to nucleic acids, such as DNA or RNA, or proteins refers nucleic acids or peptides that are substantially free of cellular material, viral material, culture or suspension medium or chemical precursors or other chemical when isolated by the methods as disclosed herein. Moreover, an “isolated nucleic acid” is meant to include nucleic acid fragments which are not necessarily naturally occurring as fragments and can not typically be found in the natural state. Accordingly, an isolated nucleic acid encompass both an isolated heterologous and/or isolated recombinant nucleic acids. The term “isolated” as used herein can also refer to polypeptides which are isolated from other cellular materials and/or other proteins and is meant to encompass both purified and recombinant polypeptides.

The term “heterologous” as used herein when used with respect to heterologous nucleic acid or heterologous protein refers to nucleic acid or protein from a different species from which it is derived. By way of a non-limiting example, a heterologous nucleic acid would be a viral nucleic acid sequence derived from a mammalian cell. Conversely, the term “homologous” when used with respect to a homologous nucleic acid or heterologous protein refers to nucleic acid or protein from the same species from which it is derived

The term “cells,” “host cells” or “recombinant host cells” are terms used interchangeably herein. It is understood that such terms refer not only to a particular cell type, but to the progeny or potential progeny of such a cell. Because certain modifications can occur in succeeding generations due to either mutation or environmental influences, such progeny can not, in fact, be identical to the parent cell, but are still included within the scope of the term as used herein

The term “subject” refers to any living organism from which a biological sample can be obtained. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “untreated biological sample” refers to a biological sample that has not had any prior sample pre-treatment except for dilution and/or suspension in a solution.

The term “elutant” or “eluted sample” as used herein refers to a sample that is collected after processing with at least one module of the microfluidic device.

The term “microchannel” as used herein, refers to a channel that is sized for passing through microvolumes of liquid.

The term “channel” as used herein means any capillary, channel, tube or grove that is deposed within or upon a substrate.

The terms “photografting” or “photoinitiated grafting” are used interchangeably herein to refer to a process wherein ultra-violet light is used to initiate a polymerization reaction that originates from the surface of the substrate that is grafter upon.

The term “o.d.” is used to refer to the outer diameter.

The term “i.d.” is used to refer to the inner diameter.

The term “Tg” as used herein refers to the glass transition temperature of a given polymer.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “enriching” is used synonymously with “isolating” cells such as, but not limited to, bacterial cells, and means that the yield (fraction) of cells of one type is increased over the fraction of cells of that type in the starting culture or preparation.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not cardiovascular stem cells or cardiovascular stem cell progeny as described herein.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the present invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Cell Lysis Device

In some embodiments, the present invention is directed to a method of manufacture of a microfluidic chip, which has channels packed with polymer-embedded particles and uses thereof. The chip of the present invention is designed for application of an untreated biological sample on the chip thus allowing lysis of cells in an untreated biological sample. In some embodiments, the chip also comprises modules for subsequent processing of the cell lysate, for example isolation, purification, and detection of biomolecules released from lysed cells that are present in the cell lysate, such as, for example nucleic acids and proteins.

In some embodiments of the present invention, the microfluidic chip allows for complete cell lysis and isolation, purification, detection and analysis of biomolecules from cells from an unprocessed biological sample in one step.

In some embodiments, the chip is a plastic-like material such as silicon. The present invention also provides a microfluidic chip for cell lysis, in particular lysis of microorganism such as bacteria, as well as plant cells and mammalian cells and microorganisms. In some embodiments, the cell lysis microfluidic chip can be combined with a microfluidic chip for isolation, purification and detection of biomolecules in the cell lysate, for example biomolecules such as nucleic acids and proteins, thus providing a complete Lab-on-a-Chip analysis system for obtaining biomolecules from cells, and their subsequent isolation, purification, detection and analysis. In some embodiments, such a Lab-on-a-chip system that can be used with the cell lysis microfluidic chip as disclosed herein is disclosed in U.S. Patent Application 2007/0015179, which is specifically incorporated herein in its entirety by reference. In other embodiments, alternative Lab-on-a-Chip systems for obtaining biomolecules from cells, and their subsequent isolation, purification, detection and analysis can be used with the cell lysis microfluidic chip of the present invention, and are commonly known in the art and are encompassed for use in the methods of the present invention.

In some embodiments, the microchip disclosed herein can be used for lysis of cells from biological samples, for example but in no way a limitation, the microchip as disclosed herein can be used for lysis of bacteria, such as gram positive bacteria and other microorganisms and plant cells present in a biological sample. In some embodiments, the microchip as disclosed herein can also comprise additional microchip modules, for example microchip modules for extraction and detection of biomolecules released from the lysed cells. For example, the microchip disclosed herein can also comprise microchips modules enabling biomolecule isolation, purification and detection from biological samples, for example where the biomolecules are, for example, nucleic acids and proteins released from the cells lysed using the microchip of the present invention.

Without being bound by theory, cell lysis is a vital step in order to extract the biomolecules contained within the cells. Lysis of a cell enables subsequent extraction, purification, and detection of the biomolecules obtained from the lysed cells, such as, nucleic acids. Nucleic acids, are for example commonly used in a number of applications for diagnostic purposes, for example nucleic acid probes can be used to detect human pathogens, food/water contaminating pathogens, plant pathogens, human or animal or plant diagnostic applications to detect polymorphisms or disease-causing polymorphisms, and pharmacogenetic applications, as well as to detect a number of genetic markers to allow development of personalized medicine. The microchip as disclosed herein can further be used to detect nucleic acids for the purpose of identifying individuals, for criminal investigations or paternity analysis. Isolation of proteins, such as antibodies or small peptides or a protein that have therapeutic value from cells, for example proteins from bacterial or plant cells is also of great importance and is also encompassed as a use for the device as disclosed herein. In some embodiments, where the microfluidic chip also comprises modules for purification and isolation of mRNA, the microfluidic chip is useful to measure gene expression or to construct a cDNA library from the lysed cells.

Accordingly, in some embodiments the present invention allows a number of different diagnostic tests to be performed from untreated biological samples, where the untreated biological samples contain cells, for example but not limited to bacterial cells, microorganisms, viruses, plant cells, mammalian cells and the like. In some embodiments, the microfluidic chip as disclosed herein can function, for example, as a portable disease surveillance device, a portable device to allow design of personalized medical interventions or identification of individuals.

In some embodiments, the microfluidic chip can also be used for simple lysis of cells in a biological sample, for example in order to harvest a biomolecule from a cell lysate, for example a protein expressed by a cell in the biological sample or alternatively for other reasons for cell lysis, such as to kill the cell.

In some embodiments, the microfluidic chip can also be used for simple enrichment of bacterial in a biological sample, for example in order to increase and/or harvest bacteria from a less concentrated sample comprising bacteria, for example bacteria in a biological sample can be collected for subsequent processing such as for cell lysis using the methods as disclosed herein.

Due to its small size, the microfluidic chip of the present invention can also provide high-speed and high-throughput cell lysis. In some embodiments where the microfluidic chip of the present invention further comprises modules for biomolecule isolation, purification, analysis, (for example, such as nucleic acid sequence detection and/or analysis), the microfluidic chip can provide high-speed and high-throughput diagnostic tests of the cells present in the biological sample.

The current commercially available cell lysis systems are macroscale systems, often requiring multiple reagents and/or depend on specific apparatus. For example the commonly used commercial Qiagen® Mini and Maxi Prep DNA extraction methods, and other commercially available DNA extraction methods. The cell lysis system on a chip of the present invention as disclosed herein is advantageous of other cell commercially available cell lysis systems in that it shrinks the conventional “bench-top” procedure into a miniature, portable device.

The microscale system of the present invention as disclosed herein is advantageous because it enables cell lysis with significantly reduced reagent consumption and also allows for lysis of a variety of different cell types present in a biological sample, for example a biological sample comprising for example a plurality of microorganisms, bacteria cells, mammalian cells, plant cells etc. In addition in some embodiments, the microscale system of the present invention enables cell lysis of a significantly reduced sample, as well as minimal exposure of the practitioner to the cells present in the bacterial sample and as well as the cell lysate. In some embodiments, the cell lysate eluted from such a cell lysis on a chip system can be further processed, for example using isolation, purification and analysis on a chip systems, enabling isolation, purification and analysis of biomolecules present in the cell lysate, such as, for example nucleic acids. In addition, the microfluidic chips of the present invention as disclosed herein are be capable of processing different biological samples in parallel, as well as biological samples comprising a variety of different cells, for example different types of bacterial cells, mammalian cells, plant cells, microorganisms and parasites etc.

As disclosed herein, the present invention is directed to a method for cell lysis using a miniature portable device. In some embodiments, the device can be used to lyse bacteria, for example gram negative and gram positive bacteria. In some embodiments, the device is capable of efficiently causing the lysis of gram positive bacteria. In particular embodiments, the device comprises a lysis column comprising a monolith which comprises carbon particles such as carbon nanotubes. In some embodiments, the carbon particles are embedded in the polymer monolith in irregular angles and in a disorganized conformation so that the carbon nanoparticles form a barbed or otherwise sharp edge or pointed edges to the surface of the polymer forming a jagged surface are for efficient cell lysis.

Previous use of monolith columns have been used on a microfluidic chip, for example, porous polymer monoliths embedded with silica particles of Klapperich et al., (U.S. Patent Application 2007/0015179) has been used as a solid-phase extraction (SPE) system to isolate nucleic acids from biological samples. However, unlike the methods and the device as disclosed herein, the microfluidic chip of Klapperich et al. in 2007/0015179 demonstrates use of crude cell lysates, where the bacterial have been chemically lysed with a lysis agent prior to applying using the polymer monolith embedded with silica particles, for the isolation of nucleic acids. The device of Klapperich et al. in 2007/0015179 is not designed for cell lysis, in particular it is not designed for the lysis of bacteria cells such as gram positive or gram negative bacteria. Other existing lab-on-a-chip devices also typically lyse the cells outside the microchip with conventional methods before the on-chip experiment, and microlitres of the lysate or purified DNA sample are loaded for the purpose of the specific chip, i.e. DNA or protein isolations chip modules. The present methods and devices as disclosed herein differs from existing lab-on-a-chip devices in that in the methods and devices as disclosed herein, cell lysis, in particular cell lysis of bacterial cells, is done on the chip without the need to pre-treat the biological samples (i.e. biological samples comprising bacterial or plant cells) for lysis, such as use of a chemical lysis agent or mechanical force lysis techniques. While some devices encompass lysis on the chip, for example Mathie et al., (International Patent Application WO04/061085) allow a method for cell lysis of bacteria on a microfluidinc analysis device using small concentrations of chemical lysis solutions and heating and cooling cycles is necessary to achieve efficient cell lysis. Unlike the device of the present invention, the device in Mathie et al., cannot achieve cell lysis without the use of extreme temperature variations and/or the use of chemical agents. Furthermore, Mathie et al., d in particular the use of carbon particles to aid cell lysis.

Furthermore, the use of a polymer monolith to entrap carbon particles as disclosed in the present invention has not been previously shown. Carbon nanotubes have been discussed as useful as a filter. Ajayan et al., (U.S. Patent Application 2006/0027499) and Srivastava et al., (Nature, 2004; 610-614) have discussed the feasibility of nanoporus nanotube filters for elimination of components from petroleum or for the use to eliminate bacterial contaminants from drinking water. However, the device of Ajayan et al. and Srivastava et al., discuss uniform dense packing of radically aligned carbon nanotubes on the walls of a channel as a filtering and separation device for eliminating bacterial and viruses from a sample, and resulting in the elutant being contaminant free. The filtering devices of Ajayan et al., and Srivastava et al., are not designed for the lysis of the bacterial contaminants, nor for the release bacterial biomolecules such as DNA and proteins into the elutant. Furthermore, the Ajayan and Srivastava approaches are essentially the opposite of the present invention, because in the Ajayan and Srivastava approaches, the biological sample is made free from the bacterial cell contamination, whereas in some embodiments of the present invention, the device is used to concentrate and/or enrich the biological sample with bacterial cells, or in alternative embodiment, release the biomolecules from bacterial cells into the elutant. Carbon nanotubes have also been used in microfluidic devices for the purpose of increasing the surface:volume (S/V) ratio in a microfluidic device (see Ricoul et al., International Patent Application WO2006/122697). In Ricoul et al. the carbon nanotubes are arranged in a substantially perpendicular way to the surface they are attached to, and are grown by a process known as PECVD (Plasma Enhanced Chemical Vapor Deposition). It is stated that in some instances, the perpendicular carbon nanotubes can have their surface functionalized by grafting chemical molecules or charged molecules to function as a catalyst, such as for digesting proteins before their analysis. However, unlike the present invention, the carbon nanotubes Ricoul et al are not barbed or otherwise positioned in a random configuration which results in sharp edged, pointed or otherwise jagged carbon particles suitable for cell lysis.

Polymer monoliths have also been discussed in microfluidic devices, for example, Frechet et al., (U.S. Patent Application 2004/0101442), however unlike the methods and devices of the present invention, the polymer monoliths in Frechet et al., are not designed to be used to lyse cells, or for embedding carbon particles. Previous uses of monolith polymers have been used to embed particles, such as monolith polymers of Oleschuck et al., (U.S. Patent Application 2006/0214099), which have particles embedded which interact with the biological sample for use in mass spectrometry or stationary phases chromatographic applications. In contrast to the present invention, the monolith polymer embedded particles in Oleschuck et al., are not used for lysis of cells but as an electrospray emitter (i.e. for emitting a sample in a spray) for mass spectral analysis and/or acting as stationary phase in chromatographic applications. Furthermore, the particles embedded within the monolith polymers in Oleschuck et al., are designed to increase the surface area which can interact with components of the sample, and are also amenable for chemical modification. Further, the monolith polymers in Oleschuck et al do not comprise carbon particles, nor any barbed or otherwise sharp edged or pointed particles or otherwise jagged particles designed for cell lysis.

One aspect of the present invention provides a microfluidic device comprising: (a) a substrate that is not glass with at least one channel of less than 150 μm in diameter, wherein the channel has an inlet, an outlet, and an internal space with a surface between the inlet and the outlet; (b) a porous polymer monolith comprising a monomer within the internal space, wherein the porous polymer monolith impregnated with carbon particles within said internal space, and optionally (c) were the interior of the channel is grafted or surface-modified to improve adhesion of the porous polymer of (b).

The channels of the microfluidic device of the present invention are typically about 50-300, and in some embodiments about 100-150 μm in diameter, and in some embodiments about 100 μm in diameter. The channels can be arranged in any manner or geometry that the skilled artisan desires. Fir example, wedge shaped, varying sizes of channels and rows. All sorts of geometric patterns are permissible. The pattern depends on the purpose to which the chip of microfluidic device is being used. The diameter may vary depend on the desired use of the product and can be easily adjusted during the process of making of the device by the skilled artisan.

In one embodiment, the microfluidic device is used for cell lysis. In an alternative embodiment, the microfluidic device is used for cell lysis and subsequent processing of the cell lysate, for example isolation and purification of biomolecules from the cell lysate (for example nucleic acids) and recovery without prior pre-treatment of the biological sample. Such an embodiment will significantly reduce the processing time and also minimize contamination of sample. Furthermore, such an embodiment where cell lysis is combined with subsequent cell lysate will take place in a completely closed environment, and thus reduce the risk of infecting the clinicians or practitioners running the process.

In some embodiments, the microfluidic chips as disclosed herein are made of plastic, and as such will be much cheaper than other microfluidic chips available in market which are made of glass or quartz.

Most currently available microfluidic devices are made of silicon and/or glass. Use of silicon and glass is relatively expensive because of high material and manufacturing costs. Polymeric materials would be less expensive. Therefore, microfluidic devices made from polymeric materials are more suitable for mass-production of disposable devices. In one embodiment, the microfluidic devices disclosed herein are made using cyclic polyolefin, such as ZEONEX® (ZEONEX 690R, Zeon Chemicals Inc. Louisville, Ky., USA).

For example, the inventors demonstrated herein that the mechanical and optical properties of cyclic polyolefins, such as ZEONEX are suitable for on-chip cell lysis.

In some embodiments, the microfluidic device disclosed herein is made of thermoplastic polymer that includes a channel or a multiplicity of channels whose surfaces can be modified by photografting. The device further includes a porous polymer monolith impregnated with carbon particles, prepared via UV initiated polymerization of a porous polymer solution embedded with the particles, within the channel.

In some embodiments, the monolith is formed by in-situ UV polymerization of a monomer mixture impregnated with for example, carbon particles. For example, one can use cyclic polyolefins. In one embodiment, the inventors demonstrated use of ZEONOR® or ZEONEX® (Zeon Chemicals, Louisville, Ky., USA), medical grade cyclic polyolefins, to manufacture a plastic microfluidic device. The inventors used ZEONEX® the primary chip material, because of its excellent mechanical properties, low auto-fluorescence and high UV transmission. However, one can use any other material with suitable optical properties can be used. The optical properties necessary for both photoinitiated polymerization during manufacturing and the integration of on-chip detection in the future include good mechanical properties, low auto-fluorescence and high UV transmission.

In one embodiment, one forms the microchannels by hot embossing with a master at about 100° C. (about 30° C. above the Tg of ZEONEX or ZEONOR) and about 250 psi for about minutes using, for example, a hot press, such as Heated Press 4386, Carver, Wabash, Ind. The master and the substrate can be manually separated at the de-embossing temperature, 60° C. Aluminum (Al) coating on the master facilitates easier removal of the master from the substrate after the embossing is completed. To seal the channels, another piece of ZEONEX or ZEONOR of the same dimensions can be thermally bonded on top, for example using 68° C., 250 psi, for 2 minutes.

In an alternative embodiment, one can prepare the microfluidic device as disclosed herein by hot embossing using wire embedded in the base plate of ZEONEX or ZEONOR substrate, as disclosed in the Examples or by using a SU-8 master. Channels of about 100 μm and about 165 μm depths can be fabricated by this method. The width of the channels can vary from about 2 μm to at least about 500 μm. The width of the channels preferably vary from about 50 μm to about 250 μm or any width between, such as about 51 μm, or about 52 μm, or about 53 μm, about 54 μm, or about 55 μm, or about 60 μm, or about 65 μm, or about 70 μm, or about 75 μm, or about 80 μm, or about 85 μm, or about 90 μm, or about 100 μm, or about 115 μm, or about 125 μm, or about 150 μm, or about 200 μm, or about 249 μm. One can drill wells of any depth. In one preferred embodiment, one drills wells of about 1.5 mm diameter at the end of the channels for sample introduction and collection.

In some embodiments, where SU-8 master is used in fabrication of the device, the SU-8 masters can be fabricated, for example, on piranha-cleaned silicon wafers by spinning SU-850 photoepoxy (Microchem, Newton, Mass.) or any other comparable method. In one preferred embodiment, one uses thickness of about 100 μm and about 165 μm onto the wafers. One then pre-bakes the wafers as is known to one skilled in the art. For example, in one preferred embodiment, one pre-baked the wafers for 30 min at 95° C. After baking, the pattern is transferred through a mask preferably, by using contact lithography. Other applicable methods may be used as is known to one skilled in the art. One follows the transfer of the pattern by development, for example with SU-8 developer (Microchem) and post-baking the wafers for, for example, 1.5 h at 175° C. In one embodiment, after the fabrication process, the SU-8 molds exhibit glass-like mechanical properties and have the negative pattern of the microfluidic channels.

In some embodiments, the wafers are sputter coated with about 500 Angstroms (Å) of titanium (Ti) for adhesion, followed by about 1000 Å of A1.

In another embodiment, one forms the microchannels by hot embossing with a master at about 100° C. (about 30° C. above the Tg of ZEONEX or ZEONOR) and about 250 psi for about minutes using, for example, a hot press, such as Heated Press 4386, Carver, Wabash, Ind. The master with and the substrate can be manually separated at the de-embossing temperature, 60° C. Aluminum (Al) coating on the master facilitates easier removal of the master from the substrate after the embossing is completed. To seal the channels, another piece of ZEONEX or ZEONOR of the same dimensions can be thermally bonded on top, for example using 68° C., 250 psi, for 2 minutes.

In one embodiment, the fabricated channels are surface-modified prior to the formation of the porous monolith to improve the adhesion of the monolith to the plastic device. This can be achieved by, for example, photografting the inner surface with a grafting monomer solution, for example comprising Methyl methacrylate (MM) and a photo-sensitizer, for example enzophenone. In some embodiments, the grafting monomer solution also comprises phethylene diacrylate (EDA). In some embodiments, the UV-initiated reaction is mediated by benzophenone. For example, one can fill the microchannels with a pre-polymer solution, for example but not limited to a mixture of MM and a hydrogen abstracting photoinitiator, such as 3% benzophenone or enzophenone. The chip can then be UV-irradiated for suitable time, for example, about 1-5 minutes, preferably 3 minutes. The grafting step can be carried out such that it leads to very low conversion and preferably also avoids the formation of crosslinked polymer within the channels. The excess monomer is preferably removed from the channels by rinsing. Rinsing can be performed, for example, with methanol at a flow rate of about 0.1 mL/min for 1 h.

In one embodiment, one forms the monolith by polymerization of a mixture a non-polar monomer, for example BUMA, and an appropriate cross linker, for example EDMA. Without wishing to be bound by theory, the permeability of the polymer monolith typically depends on its porosity. Porogenic solvents are therefore an essential part of the polymerization mixture. The porogenic solvents dissolve all the monomers and initiator to a form a homogeneous solution and control the phase separation process during the polymerization in order to achieve the desired pore structure. For example, for use with a non-polar monomer such as BUMA, a porogenic mixture of 1-dodecanol and cyclohexanol has been shown to be suitable for the preparation of non-polar porous monolithic columns. In one embodiments, one uses 2,2-Dimethyl-2-phenylacetophenone (DMPAP) as the UV initiator for non-polar porous monolithic columns.

In an alternative embodiment, one forms the monolith by polymerization of a mixture a polar monomer, for example GMA, and an appropriate cross linker, for example EDMA. As disclosed above the permeability of the polymer monolith typically depends on its porosity. Porogenic solvents are therefore an essential part of the polymerization mixture. The porogenic solvents dissolve all the monomers and initiator to a form a homogeneous solution and control the phase separation process during the polymerization in order to achieve the desired pore structure. For example, a for use with a polar monomer such as GMA, a porogenic mixture of methanol has been shown to be suitable for the preparation of polar porous monolithic columns. In one embodiment, one uses 2,2-Dimethyl-2-phenylacetophenone (DMPAP) as the UV initiator for polar porous monolithic columns.

Porous polymer monoliths are an evolution of macroporous polymers consising of beads of polymeric material connected by a cross-linking polymer resulting in gaps or pores that occur throughout the material. Pore formation is caused by the presence of a solvent system as part of the overall aqueous pre-polymer solution from which the monolith is fabricated. In recent years they have garnered interest from those interested in utilizing them in applications where it is desirable to maximize interaction between molecules flowing through the porous polymer and molecules embedded within that polymer. The porous polymer monolith provides an extremely large surface area to volume ratio, affording the opportunity to maximize molecular interaction.

Porous polymer monoliths are typically fabricated by ultra-violet, (UV), free radical cross-linking in which thermally induced decomposition of a photo-initiator causes polymerization to occur.

One can use a selection porous polymer monoliths as listed in Table 4.

One can fabricate porous polymer monoliths from a variety of pre-polymer solutions; however without being bound by theory, typically the pre-polymer solutions contain four main constituent parts; 1) The monovinyl or divinyl monomer, 2) The crosslinker, 3) The photo-initiator, 4) The solvent system.

The monomer, upon the onset of polymerization, forms the globules that comprise the bulk of the porous polymer structure. Monomers used in formulating the pre-polymer solution range from highly non-polar and hydrophobic to highly polar and hydrophilic and include butyl methacrylate, (BUMA), lauryl methacrylate, (LMA), glycidyl methacrylate, (GMA) and hydroxyethyl methacrylate, (HEMA). Monomer selection is dependant upon a variety of parameters, including desired surface chemistry and application, compatability with chosen solvents, other constituent parts and desired pore size. In one embodiment, butyl methacrylate, (BUMA) and glycidyl methacrylate, (GMA) are used in the methods of the present invention as exemplary examples of both a non-polar and polar monomers respectively.

The cross-linker is responsible for interconnecting the polymerized monomer microglobules and providing structural integrity to the monolith. In one embodiment, the cross-linker useful in the methods of the present invention is Ethylene Dimethacrylate, (EDMA) which is commonly used in the art, although other cross-linkers known in the art are encompassed for use in the methods of the present invention.

The free-radical initiator is used to initiate the polymerization within the prepolymer solution. In free-radical polymerization the initiator works by creating free radicals, (unbound electron pairs), within the monomer molecules that proceed to react with the other monomers to form chains. Eventually termination occurs, most often when one free radical reacts with another free radical to form a stable molecule. In some embodiments, the initiators useful in the present invention decompose and begin to create radicals when exposed to ultraviolet light, also referred to as photo-initiators. Examples of such photo-initiations useful in the methods of the present invention are, for example but not limited to azobisisobutyronitrile (AIBN), Benzoin methyl ether, and 2,2-dimethyl-2-phenylacetophenone, (DMPAP). One can select any photoinitiator known to one of ordinary skill in the art, although one should consider its compatibility with the selected monomer and crosslinker and the required time and energy that would be used for the photo-initiator to affect the required degree of crosslinking. In one embodiment, azobisisobutyronitrile (AIBN) is used as a photoinitiator, and in another embodiment, DMPAP is the photoinitiator useful in the methods of the present invention, particularly with the use with non-polar monomer systems³⁹.

The solvent system consists of one or more solvents and important in ensuring that pore formation occurs and determines the size and frequency of pores within the porous polymer monolith. One can use any solvent commonly known by persons or ordinary skill in the art, for example but not limited to solvent systems for use with BUMA, GMA and HEMA monomers⁴⁰. Solvents useful in the methods of the present invention can be non-polar or polar solvents, for example but not limited to cyclohexanol, dodecanol, hexane, ethylene glycol, acetic acid, propanol, ethanol and methanol. In some embodiments, the solvent system is cyclohexanol and dodecanol or ethanol and methanol, for example non-polar solvents, such as cyclohexanol/dodecanol are useful for use with non-polar monomers, for example BUMA⁴¹. In some embodiments, polar solvents, such as for example ethanol/methanol are also useful for use with polar monomers, for example GMA⁴² and BUMA.

One can then fill the surface modified chips with pre-polymer solution comprising suspended carbon particles. For example the pre-polymer solution comprising carbon particles comprises, a mixture consisting of BUMA (18% wt), EDMA (14% wt), 1-dodecanol (42% wt), cyclohexanol (10% wt), 2.27M cyclohexanol with carbon particles (10% wt) and DMPAP (1% wt with respect to monomers) is flowed through the channel.

In an alternative embodiment, the pre-polymer solution comprising carbon particles comprises, a mixture consisting of GMA (18% wt), EDMA (14% wt), methanol (40% wt) 0.033M ethanol with carbon particles (27% wt) and DMPAP (1% wt with respect to monomers) is flowed through the channel.

In an alternative embodiment, the pre-polymer solution comprising carbon particles comprises, a mixture consisting of BUMA (18% wt), EDMA (14% wt), methanol (40% wt), cyclohexanol (10% wt), 0.033M ethanol with carbon particles (27% wt) and DMPAP (1% wt with respect to monomers) is flowed through the channel.

The microchip is then preferably irradiated with UV, for example, for about 0.7 minutes per side at 1000 J and washed with, for example, methanol for 12 h at a flow rate of 0.1 mL/min.

### Types of Thermoplastic Materials for Substrates

The photografting method used in preparing the microfluidic chips of the present invention can be used for the surface modification of a wide range of thermoplastic polymers. The preferred substrates, i.e. for forming channel or tube surfaces, are selected from the group consisting of poly(methyl methacrylate), poly(butyl methacrylate), poly(dimethylsiloxane), poly(ethylene terephthalate), poly(butylene terephthalate), hydrogenated polystyrene, polyolefins such as, cyclic olefin copolymer, polyethylene, polypropylene, and polyimide. Polycarbonates and polystyrenes may not be transparent enough for efficient UV transmission and therefore may not be suitable for use as substrates.

Optical properties such as light transparency at the desired wavelength range and low background fluorescence are important characteristics of substrate materials that show potential for use in the microfluidic devices as disclosed herein. Since the photografting reactions must occur within the channels on all sides, the light must first pass through a layer of this polymer. Therefore, the substrate materials should be transparent in a wavelength range of about 200 to about 350 nm, preferably at any point in the range between about 230-330 nm such as about 250 to about 300 nm, or about 260 to about 295, etc.

In addition, the chemical properties and solubility of substrates can be taken into consideration. For instance, substrates that dissolve only in solvents, such as toluene and hexane, that are less likely to be used in standard microfluidic applications, make more desirable candidate substrate materials for photografting.

One important consideration in choosing substrate material for grafting is the grafting efficiency, expressed as Neff, of the monomer to the substrate, which depends on properties such as the chemistry and transparency for light at the desired wavelength range. Grafting efficiency values of substrates correlate well with the irradiation power, the measured values of contact angles and the transparency of the substrate. An opaque substrate with a grafting efficiency value of 0 would reflect a sample, wherein no transmitted light can be detected using the material as a filter and no grafting is achieved even after 30 minutes of irradiation.

Thickness of only a few micrometers of a UV absorbing material or solution could decrease the intensity of the UV light and, consequently, the grafting efficiency. The depth of features in typical microfluidic devices may reach several tens of micrometers. Therefore, it is important to assess the effect of UV transparency of the grafting monomer mixtures during the grafting more exactly in order to determine the depth of the channel through which sufficient grafting can be safely achieved with the chosen monomer mixture.

In general, the channel depth should be about 10-500 μm, preferably any range between about 10-250 μm including about 50-250 μm, most preferably about 10-50 μm. The thickness or width of the channel can be varied depending on the biomolecule one is looking at. For example, from about 35 μm to about 300 μm, and all ranges in between. In some embodiments, the channel ranges from about 50 μm to about 250 μm. In some embodiments, a channel can be about 100 μm depth and between about 100 μm and about 150 μm in width.

In some embodiments, wells can be prepared to introduce and collect samples at the ends of the channels. These can range from about 0.5 mm to about 2.0 mm, and all ranges in between, such as about 1.5 mm.

### Compositions of First Monomer and its Mixtures: Mixtures Used for Photografting to the Substrate to Form a Binding Surface or a Thin Interlayer Polymer

Compositions of the grafting monomer mixtures useful for photografting are generally comprised of a bulk polyvinyl monomer, a bulk monovinyl monomer, or solutions of both a polyvinyl and monovinyl monomer, in a solvent and in the presence of 0.1 to 5% photoinitiator, preferably with 10 to 30% of monomer in the solution and 0.1 to 1% of photoinitiator, even more preferably about 10-20% monomer and 0.2-0.3% photoinitiator. For example, mixtures, such as those used in the U.S. Patent Application No. US2004/0101442 can be used, which is specifically incorporated in its entirety herein by reference.

In some embodiments, the thin interlayer polymer contains unreacted double bonds, which are consequently used to covalently attach the monolith containing the carbon particles to the microchannel surface.

Suitable polyvinyl monomers for photografting the substrate include, for example but are not limited to alkylene diacrylates and dimethacrylates, alkylene diacrylamides and dimethacrylamides, hydroxyalkylene diacrylates and dimethacrylates, oligoethylene glycol dimethacrylates and diacrylates, alkylene vinyl esters of polycarboxylic acids, wherein each of the aforementioned alkylene groups consists of 1-6 carbon atoms, divinyl ethers, pentaerythritol di-, tri-, or tetramethacrylates or acrylates, trimethylopropane trimethacrylates or acrylates, alkylene bis acrylamides or methacrylamides, and mixtures thereof.

Monovinyl monomers suitable for grafting the microfluidic chips as disclosed herein include but are not limited to acrylic and methacrylic acids, acrylamides, methacrylamides and their alkyl derivatives, alkyl acrylates and methacrylates, perfluorinated alkyl acrylates and methacrylates, hydroxyalkyl acrylates and methacrylates, wherein the alkyl group consists of 1-10 carbon atoms, oligoethyleneoxide acrylates and methacrylates, acrylate and methacrylate derivatives including primary, secondary, tertiary and quarternary amine and zwitterionic functionalities, and vinylazlactones, and mixtures thereof.

In some embodiments, the monomers are selected for photografting a thermoplastic substrate selected from the group consisting of methyl acrylate and methacrylate, butyl acrylate and methacrylate, tert-butyl acrylate and methacrylate, 2-hydroxyethyl acrylate and methacrylate, acrylic and methacrylic acid, glycidyl acrylate and methacrylate, 3-sulfopropyl acrylate and methacrylate, pentafluorophenyl acrylate and methacrylate, 2,2,3,3,4,4,4-heptafluorobut-yl acrylate and methacrylate, 1H,1H-perfluorooctyl acrylate and methacrylate, acrylamide, methacrylamide, N-ethylacrylamide, N-isopropylacrylamide, N-[3-(dimethylamino)propyl]methacrylamide, 2-acrylamido-2-methyl-1-propanesulfonic acid, 2-acrylamidoglycolic acid, [2-(methacryloyloxy)ethyl]-trimethylammonium chloride, [2-(methacryloyloxy)ethyl]dimethyl(3-sulfopropyl)ammonium hydroxide, and 2-vinyl4,4-dimethyl-azlactone.

A variety of different chemistries can be used in microfluidic devices. The grafting conditions optimized for a number of monomers including perfluorinated, hydrophobic, hydrophilic, reactive, acidic, basic, and zwitterionic monomers, which cover a broad range of properties, can be used as described in the U.S. Patent Application No. US2004/0101442, which is specifically incorporated in its entirety herein by reference. Monomer groups in which the hydrogen abstraction readily occurs are also encompassed.

In some embodiments, the monomers used for grafting exhibit a grafting efficiency of 1 or close to 1. However, since the goal is to photograft the surface with the desirable chemistry, it may be preferable to use monomers that are available despite their lower grafting efficiencies to produce the desired result.

A photomask can be attached prior to photoinitiation to permit grafting only in desired areas. However, a microfluidic chip prepared using no photomasks is also encompassed.

Solubility of some photoinitiators may be poor. Its higher concentration in solution can be achieved by adding a surfactant. However, while such surfactants may be used, their use is not highly recommended for grafting the first monomer to substrates. A drawback of the addition of surfactants is that mixtures may become turbid and affect grafting. Therefore, solutions containing the initiator and the surfactant should be closely monitored for clarity and transparency. Suitable surfactants include, but are not limited to, a block copolymer surfactant such as PLURONIC®, random copolymers of ethylene oxide and propylene oxide such as UCONTM, and a polyoxyethylene sorbitan monooleate such as TWEEN®. All mixtures can be deoxygenated by purging prior to use in photografting.

Photoinitiator molecules for use in grafting monomers to thermoplastics are preferably aromatic ketones, including but not limited to, benzophenone, 2,2-dimethoxy-2-phenylacetophenone, dimethoxyacetophenone, xanthone, thioxanthone, their derivatives, and mixtures thereof.

In general, the extent of grafting can be controlled by irradiation time. Photoinitiated grafting should occur for all substrates to a low conversion. The irradiation time may vary but typically it is from 0.5 to 10 minutes, and in some embodiments the time ranges from about 2 to 5 minutes.

During photoinitiated grafting, an increase in viscosity of the monomer or its solution is observed which indicates the concomitant formation of a considerable amount of polymer in the solution. The extent of this polymerization can be reduced by diluting the monomer with a suitable solvent. Suitable solvents should be capable of solubilizing the grafted monomer. Dilution with a solvent that has lower absorbency in the UV range than the monomer itself also helps to reduce the negative self-screening effect of the monomer. Examples of suitable solvents include water, alcohols, such as tert-butyl alcohol (tBuOH), and their mixtures.

One can also use a very short, such as about 3 minutes, irradiation and reaction time to avoid the rapid crosslinking if a pure divinyl monomer is used for photografting. However, if the reaction time is not sufficient to achieve the desired extent of surface modification, the grafting time can be extended or the monomer mixture can be changed, for example, by using a 1:1 mixture of divinyl and monovinyl monomer. A monovinyl monomer used in the grafting monomer solution decreases the crosslinking density of the grafted surface layer enabling it to swell in the polymerization mixture used later for the preparation of the monolith.

### Preparation of Porous Polymer Monoliths Through Photopolymerization of Second Monomer Mixture

In some embodiments, a porous polymer monolith useful for the methods and device of the present invention is a solid polymer body containing a sufficient amount of pores of sufficient size that enable convective flow. In some embodiments, the monoliths are those as disclosed in U.S. Pat. Nos. 5,334,310; 5,453,185; and 5,929,214, the subject matters of which are hereby incorporated by reference for purposes of describing monoliths. In some embodiments, the polymer monolith is prepared by polymerizing a polyvinyl monomer or a mixture of a polyvinyl and monovinyl monomer, in the presence of an initiator, and a porogen. The polymerization mixture is added to the channel and polymerization is initiated by UV irradiation therein so as to form the polymer monolith. The polymer monolith is then washed with a suitable liquid to remove the porogen.

In some embodiments, the polymerization mixture is comprised of about 18 wt % of a polar monovinyl monomer, or 18% or a non-polar monomer, about 14 wt % polyvinyl monomer, and about 60 wt % porogens, whereby the photopolymerizations are carried out at room temperature. The ranges of each of the monomer, crosslinker and porogens can be varied according to the methods described in U.S. Pat. Nos. 5,334,310; 5,453,185; and 5,929,214, which are incorporated in their entirety herein by reference.

The polyvinyl monomer is generally present in the polymerization mixture in an amount of from about 10 to 60 wt %, and more preferably in an amount of from about 20 to 40 wt %. Suitable polyvinyl monomers include alkylene diacrylates and dimethacrylates, hydroxyalkylene diacrylates and dimethacrylates, alkylene bisacrylamides and bismethacrylamides, wherein the alkylene group consists of 1-6 carbon atoms, oligoethylene glycol diacrylates and dimethacrylates, diallyl esters of polycarboxylic acids, divinyl ethers, pentaerythritol di-, tri-, or tetraacrylates and methacrylates, trimethylopropane triacrylates and trimethacrylates, and mixtures thereof.

In some embodiments, one can use monovinyl monomers, for example but not limited to, acrylic and methacrylic acids, acrylamides, methacrylamides and their alkyl derivatives, alkyl acrylates and methacrylates, perfluorinated alkyl acrylates and methacrylates, hydroxyalkyl acrylates and methacrylates, wherein the alkyl group consists of 1-10 carbon atoms, oligoethyleneoxide acrylates and methacrylates, vinylazlactones, acrylate and methacrylate derivatives including primary, secondary, tertiary, and quarternary amine functionalities and zwitterionic functionalities, and mixtures thereof.

In some embodiments, the porogen used to prepare the monolith may be selected from a variety of different types of materials. For example, suitable liquid porogens include aliphatic hydrocarbons, esters, alcohols, ketones, ethers, solutions of soluble polymers, and mixtures thereof. The porogen is generally present in the polymerization mixture in an amount of from about 40 to 90 wt %, more preferably from about 60 to 80 wt %.

In further embodiments, the composition of porogenic solvent is used to control porous properties. The percentage of decanol in the porogenic solvent mixture with a co-porogen, such as cyclohexanol or butanediol, affects both pore size and pore volume of the resulting monoliths. A broad range of pore sizes can easily be achieved by simple adjustments in the composition of porogenic solvent. In contrast to the pore size, the type of porogen has only a little effect on the pore volume since, at the end of the polymerization, the fraction of pores within the final porous polymer is close to the volume fraction of the porogenic solvent in the initial polymerization mixture because the porogen remains trapped in the voids of the monolith.

In some embodiments, the pore size would depend on the ultimate use of the porous polymer monolith, for example the type of biological sample and/or the cell being lysed. In some embodiments, the pore size is greater than about 600 nm because this size enables flow through at a useful velocity and reasonable back pressure. However, smaller pores also may be useful and suitable for lysis of cells that are less than 1 μm in diameter.

Efficient polymerization of the porous polymer monolith is achieved by using free radical photoinitiators. In one embodiment, about 0.1 to 5 wt % with respect to the monomers of hydrogen abstracting photoinitiator can be used to create the porous polymer monolith. Typically, 1 wt % with respect to monomers of a hydrogen abstracting photoinitiator including, but not limited to, benzophenone, 2,2-dimethoxy-2-phenylacetophenone, dimethoxyacetophenone, xanthone, thioxanthone, their derivatives and mixtures thereof is used.

Surfactants, such as PLURONIC F-68, can be added to improve the solubility of photoinitiators. Suitable surfactants include, but are not limited to, a block copolymer surfactant such as PLURONIC®, random copolymers of ethylene oxide and propylene oxide such as UCONTM, and a polyoxyethylene sorbitan monooleate such as TWEEN®. All mixtures can be deoxygenated by purging prior to use in photografting.

### Carbon Particles

The solid phase of the microfluidic chip as disclosed herein can be made by in-situ by UV polymerization of the monolith column impregnated by particles, such as carbon particles. The carbon particles as disclosed herein are arranged within the polymer monolith in a barbed or substantially random and disorganized configuration, resulting in sharp or pointed or jagged edges which are suitable for cell lysis. The carbon particles are not substantially organized in a perpendicular fashion to the surface they are attached, nor are they in a radical perpendicular conformation which is useful for filtering purposes, but the carbon particles in the methods and devices of the present invention are disorganized and randomly arranged, and suitable for cell lysis.

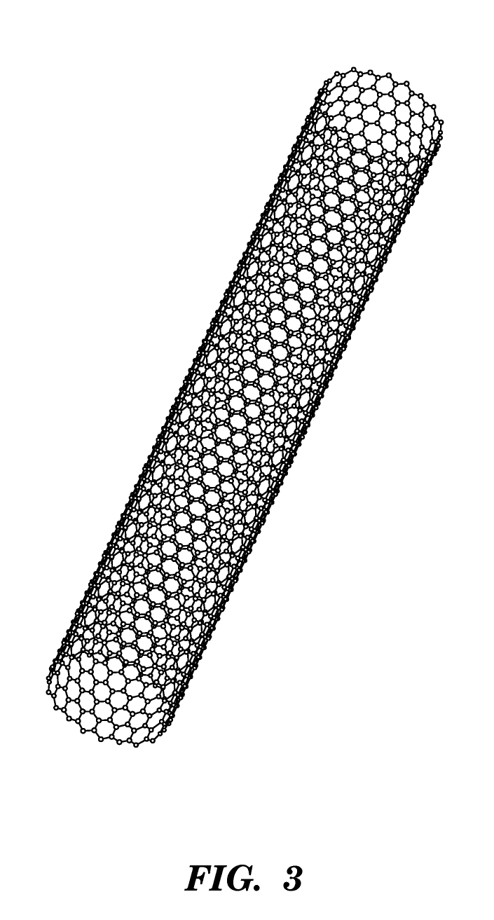

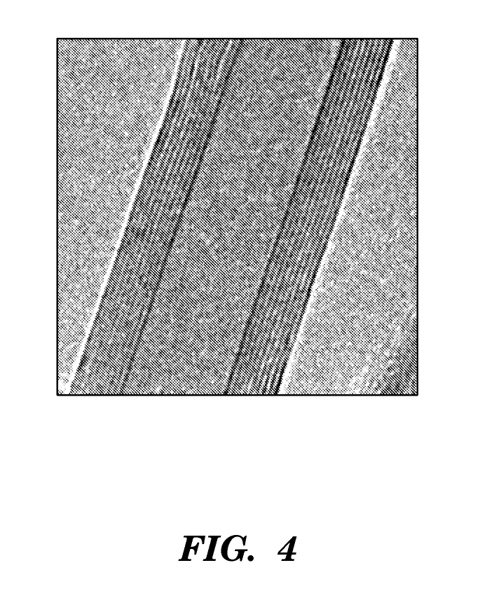

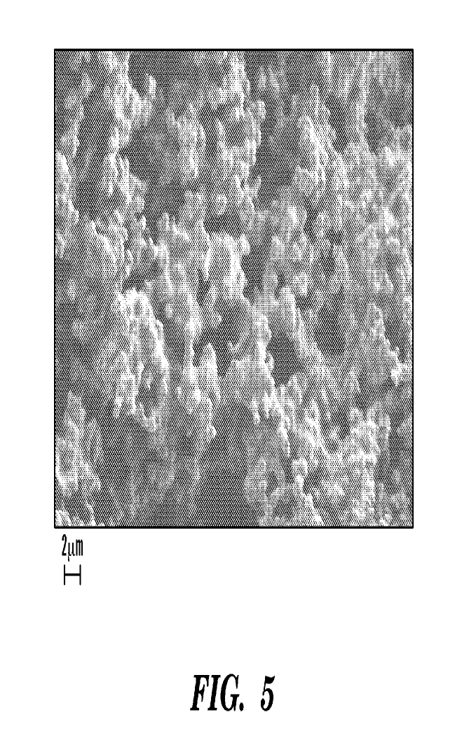

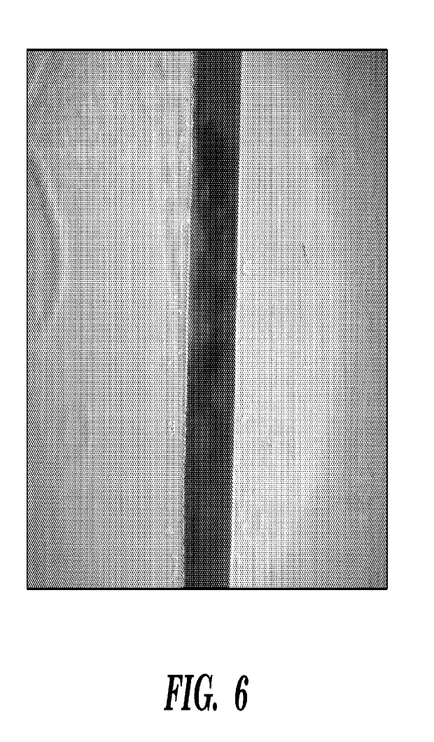

In some embodiments, carbon particles useful for the methods and devices as disclosed herein include, for example by no way a limitation, carbon nanoparticles, for example carbon nanotubes. In some embodiments, the carbon nanotubes are single walled carbon nanotubes (SWNT) as shown in FIG. 3, and in alternative embodiments, the carbon nanotubes are multi-walled carbon nanotubes (MWNT) as shown in FIG. 4.

In some embodiments, the carbon particles are carbon nanotubes. As used herein, carbon nanotubes refer to typically carbon nanotubes of about 1-50 microns, preferably about 1-20 microns, or 1-10 microns long and about 10-300 nm in diameter, preferably about 30-150 nm, alternatively about 50-150 nm in diameter.

Carbon nanotubes can be readily synthesized in gram quantities by methods commonly known in the art. Carbon nanotubes are essentially single graphite layers wrapped into tubes, either single walled (SWNT), as shown in FIG. 3, or multi walled (MWNT) as shown in FIG. 4 wrapped in several concentric layers. SWNTs are composed of a single wall of a hexagonally-bonded graphene sheet. Like the archetypal fullerene, C60, they divide space into two volumes, an inside and an outside, separated by a chemically robust, one-atom thick, impermeable membrane. The perfection of the bonding of this graphene membrane gives such fullerene carbon nanotubes outstanding properties, for example electrical conduction equivalent to metals like copper and gold, thermal conductivity along the tube axis equal to or better than that of any other material, tensile strength expected to be higher than any other material, 30-100 times higher strength than steel at one-sixth the weight, extreme stiffness combined with ability to withstand repeated bending, buckling, twisting, and/or compression at high rates with complete elasticity.

SW carbon nanotubes (SWCNT) can be produced by methods commonly known in the art, for example as disclosed in U.S. patent application Ser. No. 09/932,986, and U.S. Pat. No. 6,898,864, which are incorporated herein by reference in their entirety. In some embodiments the SWCNT can be coated with dispersal agents, for example include synthetic and naturally occurring detergents or any other compositions capable of encapsulating and solubilizing hydrophobic compounds in aqueous solutions, as described in U.S. Pat. No. 6,898,864 which is incorporated herein by reference.

One can use a variety of carbon particles based on carbon nanotubes. For example, but not limited to, in some embodiments, the carbon particles are carbon nanotubes that comprise both carbon and iron, as disclosed in U.S. Pat. No. 6,835,330, or reinforced carbon nanotubes, for example U.S. Pat. No. 6,911,260, or filled carbon nanotubes, for example U.S. Pat. No. 6,916,434 and in some embodiments the carbon particles are carbon nanotubes of varying sizes, as disclosed in U.S. Pat. No. 6,875,274, which is incorporated herein by reference in its entirety.

In some embodiments, carbon particle can be formed in-situ on the polymer monolith surface. Methods for controlled growth of carbon nanotubes are known by persons of ordinary skill in the art, such as for example but not limited to controlled growth by means of a metallic catalyst. The growth process is called PECVD (Plasma Enhanced Chemical vapor deposition) type process, where a catalyst such as nickel, cobalt or iron is used to initiate the carbon nanotube growth. For instance, such method is disclosed in U.S. Patent Application 2004/0173506, which is incorporated herein in its entirety by reference.

In some embodiments, the carbon particles is graphene. Essentially, but not being bound by theory, a carbon nanotube comprises a graphene sheet (sheet-like structure of hexagonal network of carbon atoms) rounded in a hollow form. Since a carbon nanotube shows a high electrical conductivity despite its diameter as small as 1 to 50 nm in addition to its chemical stability, it has been under extensive study for its application to devices ranging from macroscale device such as discharge electrode to nanoscale electronic device. Since a carbon nanotube itself is tough besides being electrically conductive, it has been under study for application to support for reinforcing material or structure and hydrogen storing material utilizing the action of hollow structure.

In some embodiments, the carbon particle is graphene or a graphene sheet. In alternative embodiments, the carbon particle is a “hollow graphene sheet material”, which as used herein is meant to indicate a hollowly rounded structure of graphene sheet, generally including hollow graphene sheet materials having a diameter on the order of nanometer such as straw-like carbon nanotube, conical carbon nanohorn, carbon nanobeads having bead-like carbon structures attached to straw-like carbon nanotube and helical carbon nanocoil. Such hollow graphene sheet materials are disclosed in U.S. Pat. No. 6,869,581, which is incorporated herein in its entirety by reference.

In some embodiments, the carbon nanotubes can be shortened using methods commonly known in the art, such as for example ultrasonics, and acid treatments to disperse and cleave carbon nanotubes.

In some embodiments, the carbon particle is graphite. Graphite is the closest carbon substance to carbon nanotubes that has been used in clinical studies. The development of new carbons based on graphite to considerably improve its physical properties has enabled the manufacture of endoprostheses made from carbon.

In some embodiments, the carbon particles are surface treated, also termed herein as “funtionalization” prior to adding to the monolith mixture. For example, some carbon particles, for example carbon nanotubes or multi-walled carbon nanotubes are partly immiscible within the non-polar solvents and BUMA monomer pre-polymer solution, resulting in clumping and aggregation of the carbon nanotubes and falling out of suspension. Accordingly, in some embodiments, one can perform a surface treatment of the carbon particles, for example carbon nanotubes along with ultrasonication of the carbon particles in the solvent mixture, for example ultrasonication of nanotube/solvent mixture to facilitate the generation of an effective and stable suspension.

In some embodiments, surface treatment of the carbon particles can be performed using chemical oxidation via refluxing the carbon particles, for example carbon nanotubes in 1:3 nitric and sulfuric acid⁴³. In some embodiments, surface treatment of the carbon particles, for example the carbon nanotubes is performed by refluxing the carbon particles at 140° C. for 1 hr in 1:3 nitric and sulfuric acid, followed by mix-cooled for 10 minutes, after which the carbon particles for example carbon nanotubes are extracted, for example using a sintered glass filter. In some embodiments, following surface treatment, the carbon particles for example carbon nanotubes are washed with purified water until a pH of about pH 7. One can also dry the carbon particles, for example carbon nanotubes using a vacuum dried and recollected in powder form prior to adding to the monolith pre-polymer solution.

In some embodiments, the surface treatment carbon particles, for example carbon nanotubes are further suspended in cyclohexanol and ultrasonicated. In some embodiments, the ultrasonification is for approximately 30 minutes, or up to 60 minutes or longer at a 50% duty cycle. In some embodiments, the carbon nanotubes are suspended at a concentration of 0.0025M to 0.25M prior to ultrasonicating the cyclohexanol/MWNT suspensions.

In some embodiments, functionalization can be performed using small percentages of detergents and polyols commonly found in consumer products such as glycerol, PEG-60, benzyl alcohol etc. to the nanotube suspensions. Another approach consists of using functionalized SWNT as these are readily soluble in polar solvents.

Polymerization of the Channel-Filling Porous Polymer with Particles

One can prepare the monolith as disclosed herein, and in addition, mix the monolith mixture with carbon particles. Such carbon particles, for example carbon nanotubes can easily be used to impregnate the internal space of at least a part of a channel of the microfluidic device of the present invention.

One can then fill the surface modified (i.e. grafted channel) microfluidic channel with a polymer monolith comprising carbon particles. In some embodiments, the pre-polymer solution comprising carbon particles is a mixture comprising, for example but not limited to, BUMA (18% wt), EDMA (14% wt), 1-dodecanol (42% wt), cyclohexanol (10% wt), 2.27M cyclohexanol with carbon particles (10% wt) and DMPAP (1% wt with respect to monomers) is flowed through the channel. In an alternative embodiment, the mixture comprises GMA (18% wt), EDMA (14% wt), methanol (40% wt) 0.033M ethanol with carbon particles (27% wt) and DMPAP (1% wt with respect to monomers) is flowed through the channel. In a further embodiment, the mixture comprises BUMA (18% wt), EDMA (14% wt), methanol (40% wt), cyclohexanol (10% wt), 0.033M ethanol with carbon particles (27% wt) and DMPAP (1% wt with respect to monomers) is flowed through the channel. Alternatively, the monomer mixture may further comprise a solvent.

The microchip is then preferably irradiated with UV for about 0.75-2 minutes and washed with, for example, methanol for 12 h at a flow rate of 0.1 mL/min.

The skilled artisan can readily alter the composition of the mixture used for generating a porous monolith comprising carbon particles of the device as disclosed herein based upon the present description and examples. Accordingly, any suitable modification of the mixture can be used according to the teachings of the present invention.

The skilled artisan can also readily alter the way and pattern the microfluidic channel is filled with the porous monolith embedded with carbon particles. For example, one can fill the interior space of the microfluidic channel of the device 100% with the porous polymer embedded with carbon particles and in alternative embodiments, the internal space of the microfluidic channel of the device is not completely filled porous polymer embedded with carbon particles, for example the internal space of the microfluidic channel is filled, for example, about 90%, or about 80%, or about 70%, or about 60%, or about 50% or less than 50% with the porous polymer embedded with carbon particles. In the regions not comprising the porous monolith embedded with carbon particles, the regions can comprise a porous polymer without carbon particles, or can comprise any other material or the regions can not comprise any material (i.e. the regions are void, or not filled with anything). The regions not comprising the polymer monolith comprising carbon particles can be continuous or non-continuous, for example the polymer monolith comprising carbon particles may be sandwiched or flanked between regions of polymer monolith without carbon particles, or alternatively, vice versa, a polymer monolith without carbon particles may be sandwiched or flanked between regions of polymer monoliths comprising carbon particles. Accordingly, any suitable filling of the microfluidic channel with the porous polymer monolith comprising carbon particles can be used according to the teachings of the present invention

In some embodiments, the polymer monolith comprising carbon particles may comprise more than one type, for example but not limited to a combination of carbon nanotubes, SWNT, MWNT, filled nanotubes, graphene, hollowed tube graphene and the like, and the carbon particles can be of heterogeneous sizes or the same size. Such embodiments are useful for cell lysis of cells within a biological sample when the biological sample comprises cells of varying sizes and resistance to lysis. The skilled artisan can also readily alter the type and number of different types of the various carbon particles embedded the porous polymer monolith as disclosed herein based upon the present description and examples. Accordingly, any suitable geometric format can be used according to the teachings of the present invention.

The monomer mixture is deaerated and then pumped to fill the pores of the monolith. The mixture is generally comprised of a bulk monomer or its 10 to 50% solution in a solvent and 0.1 to 5% photoinitiator, preferably 10 to 30% of monomer in the solution and 0.1 to 1% of photoinitiator.

Grafting is preferably achieved by UV irradiation of a stationary porous monolith filled with the monomer/particle solution through a mask from a sufficient distance for a sufficient period of time to graft polymer chains having functional groups to the monolith. When the irradiation step is complete, the capillary is then washed to remove residual monomer solution. Any solvent that dissolves the residual polymer can be used to wash the capillary.

Suitable monomers for photografting porous polymer monoliths impregnated with particles, possess a variety of functionalities, but are in no way limited to, hydrophilic, hydrophobic, ionizable, and reactive functionalities.

Examples of suitable monomers for photografting porous polymer monoliths include, but are not limited to, methyl acrylate and methacrylate, butyl acrylate and methacrylate, tert-butyl acrylate and methacrylate, 2-hydroxyethyl acrylate and methacrylate, acrylic and methacrylic acid, glycidyl acrylate and methacrylate, 3-sulfopropyl acrylate and methacrylate, pentafluorophenyl acrylate and methacrylate, 2,2,3,3,4,4,4-heptafluorobutyl acrylate and methacrylate, 1H,1H-perfluorooctyl acrylate and methacrylate, acrylamide, methacrylamide, N-ethylacrylamide, N-isopropylacrylamide, N-[3-(dimethylamino)propyl]methacrylamide, 2-acrylamido-2-methyl-1-propan-esulfonic acid, 2-acrylamidoglycolic acid, [2-(methacryloyloxy)ethyl]-trim-ethylammonium chloride, [2-(methacryloyloxy)ethyl]dimethyl(3-sulfopropyl)ammonium hydroxide, and 2-vinyl-4,4-dimethyl-azlactone.

Solubility of some photoinitiators may be poor. Its higher concentration in solution can be achieved by adding a surfactant. However, use of surfactants is not highly recommended. A drawback of the addition of surfactants is that mixtures may become turbid, and thus not allow irradiation with UV light and affect grafting. Therefore, solutions containing the initiator and the surfactant should be closely monitored for clarity and transparency.

In some embodiments, the desirable solvent for use in photografting polymer monoliths (i) should not absorb excessively in the UV range to exert minimum self-screening effect, (ii) should not allow hydrogen abstraction, thereby being incorporated into the polymer layer by termination reactions and/or initiate undesired homopolymerization, and (iii) must dissolve all components of the third monomer mixture (monomer and initiator). In one embodiment, the solvent is water, t-butanol (tBuOH) and its mixtures with water, that all meet these criteria.

In some embodiments, functionalization by photoinitiated grafting of porous materials located within capillaries, microfluidic channels, and other suitable devices is performed. Functionalization permits porous polymer monoliths within the capillaries and channels of microfluidic and other devices to be used for various procedures such as mixing, concentrating, cell lysis, collecting and separation reactions. Thus, in some embodiments, the design and preparation of numerous functional elements that are instrumental to the development of a cell lysis system, and optionally downstream complex microanalytical elements and systems is also done.

Furthermore, a major advantage of the microfluidic chips and methods described herein is the ability to pattern grafted areas thus facilitating preparation of materials with different spatially segregated chemistries within a single porous polymer monolith for cell lysis. Functionalization of several areas can be controlled in terms of placement and extent as simultaneous or sequential functionalizations are possible.

The additional benefit of photoinitated grafting is the ability to create patterns differing in properties such as surface coverage or type of the grafted chemistry. By placing masks over certain areas of the porous polymer monolith, patterns of different functionalities can be created. The sharp edges of the patterned features enable placing different functionalities within a porous polymer monolith next to each other with no dead volume between the functionalities, thereby allowing different elements to be placed directly adjacent to each other. In contrast to the typical “homogenous” grafting, the preparation of monoliths with longitudinal gradients of surface coverage or combining different chemistries using masks with a gradient of transparency for UV light is also contemplated by the invention.

Photografting also facilitates the preparation of layers of functionalities in a porous polymer monolith in both axial and radial direction with respect to the direction of flow.

The qualitative effect of the intensity of the UV light on the grafting efficiency is different polymers can be used as filters to modulate intensity. The use of a photomask, such as a multi density resolution mask (Series I, Ditric Optics, Hudson, Mass.), that includes several fields differing in UV light transmittance enables creation of creation of gradients. Grafting through masks with a gradient of absorbency enables the fabrication of layers with both stepwise and continuous gradients of hydrophilicity, polarity, acidity, or combinations thereof, along the channel by simply using multidensity, continuous gray-scale photomasks, a moving shutter or the like.

One of the reasons for the photografting surfaces of thermoplastic substrates is to modify the walls of channels in microfluidic devices to hold porous polymer monoliths. Any known photografting methods can be used. The channel walls in a microfluidic chip are preferably photografted as described in the U.S. Patent Application No. US2004/0101442, which is specifically incorporated in its entirety herein by reference, to achieve a firm covalent bond between the channel wall and porous polymer monoliths. This method described herein prevents the formation of voids at the monolith-wall interface.

In some embodiments, the chip can be prepared by hot embossing with an SU-8 master as described in U.S. Patent Application US2007/0015179, which is incorporated herein by reference. Prior work in hot embossing microscale features into polymeric substrates used nickel alloy molds made with LIGA or electroforming, which can be very cost intensive. In some embodiments, a rapid prototyping process is used which involves embossing directly from the SU-8 master. In such an embodiment, the chips fabricated by the hot embossing process were then used for on-chip cell lysis of cells in a biological sample. In some embodiments, the chip is made by hot embossing with a mold under high temperature and pressure. The mold itself can be made by LIGA, metal electroform made by electroplating, etching glass or silicon, epoxy based photoresists such as SU-8, and CNC milling of a metal piece. In one embodiment, one uses SU-8 molds and etched silicon molds, since they are the most inexpensive techniques. For large production of the device, other methods such as metal electroform or LIGA is more applicable. The device can also be made by injection molding of the same polymer material.

Surface modifying the interior of the walls of the microchannels for attachment of polymer monolith.

Due to the relatively inert properties of the polymeric channel surfaces, it is difficult to achieve good bonding of the solid phase with the native walls of the plastic devices. Silane primer reagents, such as 3-(trimethoxysilyl)propyl methacrylate, can be easily used to functionalize the walls of the channels made in glass or silicon. However, no such surface primers are readily available for pretreatment of polymer surfaces, so other surface modification methods, such as polymer grafting have to be applied. In our case, the grafting was done via photoinitiated polymerization prior to the formation of the monolith. The grafted interlayer polymer covalently attaches to the monolith and prevents the formation of voids between the monolith and the channel surface. The interlayer also stops the monolith from migrating down the channel during separations. The high UV transmission of ZEONOR makes it suitable for in-situ photopolymerization applications. Photopolymerization of monolith embedded with silica particles is an easy alternative to the widely-used silica bead/sol-gel approach. Stachowiak et al. demonstrated the formation of polymer monolith inside of a cyclic olefin polymer.

Material selection: Any engineering polymer that satisfies the following criteria can be used to make the device. The polymer should be compression moldable, it should not be excessively autofluorescent, and it should be transparent to UV light for easy curing of the solid phase and transparent at 488 nm and 530 nm for conventional detection methods

There are several commercial engineering polymers that meet these criteria such as polymethyl methacrylate (PMMA), polycarbonate (PC), and several proprietary cyclic olefin materials (such as ZEONOR and ZEONEX). Cyclododecatriene A high-purity, liquid cyclic polyolefin, DuPont; Cyclododectriene (CDDT), a high purity, liquid cyclic Polyolefin, CAS Number: 4904-61-4; (poly(methyl methacrylate)), or cyclic polyolefin; cyclic polyolefin polymer (ZEONEX), ZEON corporation.

### Uses of the Microfluidic Device

### Lysis of Cells

The inventors have developed a methods of lysing cells, for example lysing mammalian cells, microorganisms, plant cells and bacteria, for example gram-positive and gram-negative bacteria. The method involves using a disposable plastic microfluidic device as disclosed herein comprising a microfluidic channel comprising a polymer monolith embedded with carbon particles, for example carbon nanotubes. The carbon-particle embedded column of the microfluidic device of the present invention is capable of lysing cells present in an untreated biological sample of about 100 microliters or less. This technological development, when combined with parallel progress in chip-based biomolecular isolation and analysis, for example nucleic acid isolation and subsequent polymerase chain reaction and fluorescence detection provides a superior differential diagnosis of numerous micro-organism and/or bacterial infections at the point of care.

Sample solution volumes vary, but are in the range of about 50 mL to about 1000 microliters (μl) or greater than 1000 μl. In some embodiments, the sample volume is in the range of about 10 μl to about 1000 μl and most preferably in the range of about 50 μl to about 100 μL. Components (such as chemicals or bacteria) within the sample solution can be in the concentration of about 1.0×10⁻¹⁸ M to about 11.0×10⁻² M, and in some embodiments from about 1.0×10⁻¹⁶ M to about 1.0×10⁻⁴ M. In some embodiments, the sample can comprise bacteria within the sample of about 1.0×10 CFU/ml to about 1.0×10¹⁰ CFU/ml. In some embodiments, the concentration of bacteria in a sample is, about 1.0×10 CFU/ml, or about 1.0×10²CFU/ml, or about 1.0×10³CFU/ml, or about 1.0×10⁴CFU/ml, or about 1.0×10⁵CFU/ml, or about 1.0×10⁶CFU/ml, or about 1.0×10⁷ CFU/ml, or about 1.0×10⁸CFU/ml, or about 1.0×10⁹CFU/ml, or about 1.0×10¹⁶CFU/ml or greater than or about 1.0×10¹⁰CFU/ml. The loading flow rate can range from about 1 μl/min to about 500 μl/min.

Suitable flow rates of the methods and device as disclosed herein include rates in the range of about 1 μl/min to about 500 μl/min, more preferably in the range of about 2 μl/min to about 100 μl/min, and in some embodiments the flow rate can be about 500 μl/hour, for example, between the range of about 5 μl/min to about 10 μl/min.

Pressure can also be applied to cell lysis column of the device as disclosed herein to aid the flow of the sample through the column. In some embodiments, the pressure applied can be by a syringe, or alternatively, a mechanical pump can be used. In some embodiments, pressures are applied sufficient to force the biological sample through the column to result in cell lysis. In some embodiments, the pressures in the range of about 20 to about 8000 psi, or in the range about 100 to about 4000 psi, or in the range about 300 to about 1500 psi.

The amount of pressure used to pump the biological sample through the monolith with entrapped carbon particles is proportional to the length of the path through the composition, i.e., the amount of monolith with entrapped carbon particles through which the sample passes. Generally speaking, the longer the path, the higher the pressure.

Stachowiak et al. (Electrophoresis 24, 3689-93, 2003) demonstrated the formation of a polymer monolith within a cyclic olefin polymer. However, the use of the polymer monolith to entrap carbon particles has not been previously shown. The channel walls are modified by a polymer photografting method to encourage formation of covalent bonds with the monolith. The technique allows successful lysis of cells within an unprocessed biological sample, for example a crude biological sample.

Existing on-chip diagnostic devices typically lyse cells outside the microchip with conventional methods before the on-chip experiment, and microliters of the cell lysate or purified DNA sample were loaded onto the chip for DNA isolation the present invention differs from the existing methods. The present invention provides a method for cells lysis on the chip without the need for pretreating the sample. Typically, only samples that are not fluid enough to be applied through the inlet of the channel of the cell lysis channel device as disclosed herein may need to be mixed with a buffer before application of the sample into the channel. For example, one can use chaotropic agents followed by addition of biological sample suspended in a chaotrophic buffer to the device as disclosed herein. In alternative embodiments, the biological sample can be optionally mixed with a chaotrophic buffer in a mixing well or reservoir on the device prior to or during passing of the biological sample through the channel comprising polymer embedded carbon particles.

The inventors have demonstrated they can successfully lyse cells from a variety of bacterial strains with distinct characteristics, for example gram-positive and gram-negative bacteria, for example E. coli and C. Difficile and B. Subtillis, and that the resulting cell lysates can be used for isolation of nucleic acids and other biomolecules.

In the unlikely event that cell walls, for example bacterial cell walls or plant cell walls plug an carbon particle embedded polymer monolith column that has pores that are too small, it is also envisioned to fabricate a range of columns using different amounts of porogen, and thus different sizes of pores.

Typical cell lysis procedure using the device comprises the following steps: 1) Obtain a biological sample; 2) Optionally, culture cells in the biological sample at appropriate temperature, for example at 37° C., in an appropriate culture medium; 3) Optionally, suspend the cells from the biological sample, for example bacterial cells in an appropriate buffer system; 4) Run the bacterial sample through the microfluidic polymer monolith embedded with carbon particles; 5) collect the cell lysate from the end of the column in a collection well or reservoir. In some embodiments, the cell lysate can be immediately further processed, where the cell-lysis device also comprises additional modules, for example biomolecule isolation and analysis, the method comprising passing the cell lysate from the cell lysate module of the device over 6) a SPE-column as disclosed in U.S. Patent Application 2007/0015179, and 7) washing such a SPE column, 8) extracting isolated nucleic acids; 9) Remove isolated and concentrated nucleic acids from chips; 10) Run polymerase chain reactions using primers designed to detect a nucleic acids present in the bacteria to be detected.

In some embodiments, the bacterial sample is passed over the microfluidic polymer monolith embedded with carbon particles under slight pressure. One can use any means to apply pressure to force the biological sample over the lysis column comprising polymer monolith embedded with carbon particles, for example using mechanical force such as, for example use of a syringe as disclosed in the Examples, or using a electrical system to apply pressure. Accordingly, any suitable means to apply pressure can be used according to the teachings of the present invention.

One can lyse cells present in a biological sample, for example eukaryotic and prokaryotic cells and bacterial cells using the device of the present invention. In some embodiments, the cells in the biological sample are suspended in an appropriate buffer, for example, but not limited to a buffer comprising a detergent. Examples of detergents include, but are not limited to synthetic or natural detergents. Such detergents are commonly known by persons of ordinary skill in the art, and include, for example but not limited to detergents or any other composition capable of encapsulating and suitably solubilizing hydrophobic compounds in aqueous solutions. Exemplary detergents are, for example without limitation, synthetic or naturally occurring detergents having high surfactant activities such as detergents having a hydrophilic-lipophilic balance value no greater than about 13.2, octyl-phenoxypolyethoxyethanol (commonly referred to as Nonidet P-40 or (NP-40), polyoxyethylene sorbitol esters (e.g., TWEEN® and EMASOL™ series detergents), poloxamers (e.g., the Pluronic™ series of detergents and Poloxamer 188, which is defined as HO(C₂H₄O)(a) (C₃H₆O)(b)(C₂H₄O)αH, with the ratio of a to b being 80 to 27 and the molecular weight being in the range of 7680 to 9510) and ammonium bromides and chlorides (e.g., cetyltrimethylammonium bromide, tetradecylammonium bromide and dodecylpyrimidinium chloride), naturally occurring emulsifying agents such as deoxycholates and deoxycholate-type detergents (e.g., taurocholic acid), sapogenin glycosides (e.g., saponin) and cyclodextrins (e.g., α-,x3b2- or γ-cyclodextrin), chaotropic salts such as urea and guanidine, and ion pairing agents such as sulfonic acids (e.g., 1-heptane-sulfonic acid and 1-octane-sulfonic acid).

In some embodiments, the buffer is a chaotropic buffer, and in some embodiments, it comprises, for example guanidinium thiocyanate.

In some embodiments, the cell lysis device of the present invention comprises a mixing well, and in some embodiments, the cell lysis comprises a mixing channel, for example but not limited to a mixing channel in the form of a serpentine mixing channel that can adequately mix the sample with the lysis agent. It is specifically noted that the present invention is not limited to any particular shape of the device or the channels. The skilled artisan can readily alter the geometrics of the device based upon the present description and examples. For example, the cell lysis device may have an input for the biological sample and an input for a lysis buffer, such that they mix in the mixing channel or mixing well. Accordingly, any suitable geometric format can be used according to the teachings of the present invention.

### Use of the Device for Enrichment Purposes

In some embodiments, the methods and cell lysis device of the present invention can be used for enrichment of bacterial cells within a biological sample. For example, the methods and cell lysis device as disclosed herein can be used to increase and/or harvest bacteria from a less concentrated sample comprising bacteria, for example bacteria in a biological sample can be collected for subsequent processing such as for cell lysis using the methods as disclosed herein.

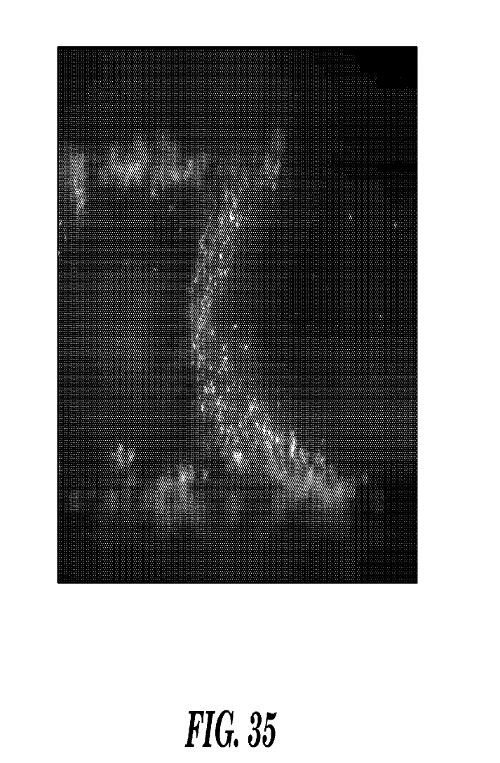

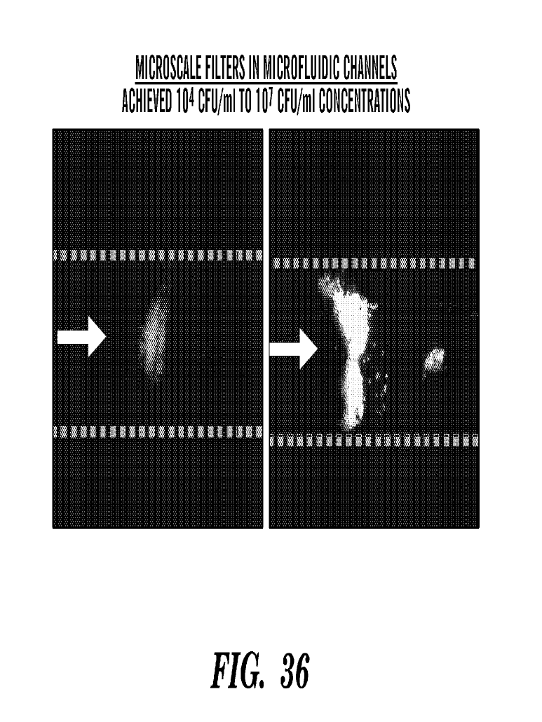

As shown in FIGS. 35 and 36, bacterial cells can be collected in front of the monolith filter, with the bacteria on the left, which can then be recovered and collected from the filter to enrich the concentration of the bacteria in a sample. Recovery of the biological sample can be collected from the filter and then, in some embodiments, optionally processed via the microfluidic channel comprising the carbon particle embedded monolith as disclosed herein, for bacteria lysis. In some embodiments, the concentration of bacteria can be enriched, for example from about 10⁴ CFU/ml to at least about 10⁷ CFU/ml of bacteria. In some embodiments, the bacteria can be enriched from about 10² CFU/ml to about 10⁹ CFU/ml or more as compared to the starting bacteria concentration in the biological sample.

Accordingly, one can enrich for bacteria of a specific type, for example based on the filter composition and pore size diameter, one can enrich for bacteria of a certain size, while allowing bacteria cells of a smaller size to go through the filter and be subsequently processed through the cell lysis column as disclosed herein. Alternatively, in another embodiment, the collected bacterial can be collected from the filter and subsequently processed by the cell lysis column as disclosed herein. In such embodiments, the elutant from the lysis step using the cell lysis column as disclosed herein would comprise a substantially pure population of biomolecules from the cells collected and harvested by the filtration step proceeding cell lysis step.

In some embodiment, filters which can be used in the methods and devices as disclosed herein are known by persons of ordinary skill in the art, such as microporus membranes available from Milipore Corporation, or membrane filters as described in U.S. Pat. No. 4,203,848 for polyvinylidine fluoride (PDVF) and U.S. Pat. No. 4,340,479 for polyamide membranes, which are both incorporated herein in their entirety by reference. Variations on such filters can be used, for example but are not limited to; asymmetric membranes, as disclosed in U.S. Pat. No. 4,629,563; membranes with a large pore surface area such as in U.S. Pat. No. 4,261,834, which are both incorporated herein in their entirety by reference; multiporus multilayered membranes structures as disclosed in the following U.S. Pat. Nos. 5,228,994; 4,770,777; 5,550,167; 5,620,790 and 5,620,790 which are both incorporated herein in their entirety by reference. Other filters which can be used for enriching the bacteria prior to cell lysis using the methods and device as disclosed herein are filters as disclosed in U.S. Pat. Nos. 7,229,665 and 5,444,097 which are incorporated herein in its entirety by reference.

In some embodiments, the filter for enriching the bacteria prior to cell lysis using the methods and device as disclosed herein is an asymmetrical membrane with a pore gradient from about 2:1 to about 1000:1, preferably from about 2:1 to about 100:1. This asymmetry is measured by comparing the average pore size on one major surface of the layer with the average pore size of the other major surface of that layer. In some embodiments, the filter membrane useful in the enriching step as disclosed herein has two or more asymmetrical layers, each having a different or if desired, similar asymmetry.

Additionally, one can vary the thickness of each layer within a wide range and still contain a self-supporting integral multilayered structure. In some embodiments, the membrane filters as disclosed herein has a thickness of between about 50 and 200 microns for good filtration and support, however, in some embodiments, the thickness of one layer can be as thin as 10 microns. In some embodiments, one can use a 150 micron thick membrane that can have a first layer that is from about 10 to about 140 microns thick, while the other is correspondingly from about 140 microns to about 10 microns in thickness.

### Use of the Device for Sterilization Purposes

In some embodiments, the methods and cell lysis device of the present invention can be used for sterilization of a biological sample, for example the cell lysis device can be used to lyse microorganisms, for example bacterial cells contaminating a biological sample. Accordingly, the cell lysis results decreasing viable microorganisms, for example bacteria, in a biological sample and thus functions a sterilization method. In some embodiments, the cell lysis device of the present invention is useful for sterilization of a small sample of a biological sample, effectively the methods and cell lysis device as disclosed herein can be used for micro-sterilization or sterilization on a micro-scale.

### Addition of Other Modules to the Cell Lysis Device

As disclosed herein, the cell lysis device of the present invention can be used on its own, or in some embodiments it can optionally comprise additional modules for biomolecule isolation, purification and analysis. In one embodiment, the biomolecules from cell lysate eluted from the cell lysis module are nucleic acids, for example DNA. One such module that can be added to the cell lysis module is, for example but not limited to, a microfluidic device for isolating DNA comprising a solid phase extraction (SPE) column that is capable of binding, concentrating and eluting nucleic acids from cell lysate samples of about 100 microliters or less. Such a microfluidic device is disclosed in U.S. Patent Application 2007/0015179 which is specifically incorporated herein in its entirety by reference. In some embodiments, the device can optionally comprise additional mixing wells and inlet areas for addition of a buffers required for the methods for biomolecule isolation, purification and analysis.

The isolation of nucleic acids can be done with a solid-phase extraction system formed by trapping silica particles in a porous polymer monolith. After the lysate flows over the solid-phase, wash buffer (2-propanol/water) will be passed through the device to remove the proteins that adsorb onto the silica. Finally, the nucleic acids will be eluted in a low stringency buffer.

Solid phase extraction (SPE) is an important and widely used sample preparation technique, which allows both the purification and preconcentration of biological samples. The purification of nucleic acids is usually done with solid-phase extraction on silica resins. Extraction is achieved because nucleic acids have the tendency to bind to silica in the presence of a high concentration of chaotropic salt. The extracted nucleic acids are subsequently eluted in an aqueous low-salt buffer and concentrated into a very small volume. The time necessary for nucleic acid purification was greatly reduced when the original phenol extraction method was replaced by silica based solid-phase extraction systems. SPE methods for DNA extraction have since been successfully miniaturized and incorporated in microfluidic chips. The sol-gel/silica bead mixtures have been shown to have very good extraction efficiencies and reproducibility in microfluidic systems. However, the sol-gel process involves high temperatures and is not suitable for use in polymeric devices.

The method of immobilizing silica particles in a porous polymer monolith to form a microscale solid-phase extraction system is described, supra. Monolithic materials have been successfully used in a wide variety of applications, including capillary electrochromatography, micro-mixers and electroosmotic pumps. The monolithic column was formed by in situ UV polymerization of a monomer mixture impregnated with silica particles. The solid-phase was covalently attached to the walls of the microchannels to prevent its displacement when samples were flowed through the channels.

Solid phase extraction (SPE) allows both the purification and preconcentration of biological samples (Weeks, B. L., et al., Scanning, 2003. 25(6): p. 297-9). The purification of nucleic acids is usually done on silica resins (Breadmore, M. C., et al., Electrophoresis, 2002. 23(20): p. 3487-95). Extraction is achieved because nucleic acids will bind to silica in the presence of a high concentration of chaotropic salt. The extracted nucleic acids are subsequently eluted in an aqueous low-salt buffer and concentrated into a very small volume. SPE methods for DNA extraction have been successfully miniaturized and incorporated in microfluidic chips. The sol-gel/silica bead mixtures have good extraction efficiencies and reproducibility in microfluidic systems (Breadmore, M. C., et al., Towards a microchip-based chromatographic platform. Part 1: Evaluation of sol-gel phases for capillary electrochromatography. Electrophoresis, 2002. 23(20): p. 3487-95; Breadmore, M. C., et al., Anal Chem, 2003. 75(8): p. 1880-6). However, the sol-gel process involves high temperatures and is not suitable for use in polymeric devices.

In another embodiment, another module that can be added to the cell lysis module is, for example but not limited to, an analysis module, for example an immunoassay module for analysis of proteins and peptide present in the cell lysate eluted from the cell lysis module. In one embodiment, such a module is, for example but not limited to a microfluidic device comprising an immunoassay, for example as disclosed U.S. Patent Application 2007/0015179 which is specifically incorporated herein in its entirety by reference.

### Uses of the Cell Lysis Device for Diagnostic Purposes

Since 1983, PCR has allowed not only for the detection of an infectious agent but also for its identification through the amplification of specific molecular markers. The advent of microfluidic technology in the early 1990's held the promise of easy to use, minimally invasive, point-of-care diagnostic devices that exploit molecular techniques. In fact, many biochemical methods including separations of proteins, nucleic acids, and performance of PCR have been miniaturized in the research lab as successful proofs of concept. By obviating the need for a full diagnostic laboratory, advanced, specialized laboratory tests once thought impractical or too costly to perform in remote areas, field hospitals, and small clinics will become routine.

Simple, inexpensive diagnostics will have an impact in several broad areas of general interest, such as homeland security, differential diagnosis in nursing homes and hospitals, in remote low income areas and the developing world. Many agents considered likely for use in a biological attack against military or civilians present with common symptoms in the clinic. Only after close observation of the first few “beacon” cases will clinicians be able to conclusively diagnose the presence and nature of a biological attack. The time lost in making these distinctions using traditional diagnostic techniques that require a full scale laboratory and skilled labor will likely lead to spread of an outbreak before containment procedures can be initiated. In addition, many antibiotic treatments are most effective if they are initiated before the onset of major symptoms.

Common difficult-to-diagnose infections are responsible for hundreds of thousands of deaths in the U.S. each year. For example, based solely on the symptoms, it is virtually impossible to know whether a diarrheal illness will have a progressive and/or fulminant course. Thus, the availability of a simple, rapid, low-cost, sensitive and specific diagnostic test would permit the delivery of directed treatment for many acute diarrheas. A case in point is colitis due to Clostridium difficile. C. difficile is the most common cause of diarrhea spread in hospitals and nursing homes in the United States and is increasingly a major cause of morbidity and mortality among the elderly in acute and chronic healthcare facilities. Ideally, additional antimicrobial therapy should be initiated early, but no sensitive, specific, and reliable test exists for making a diagnosis of C. difficile associated diarrhea at the initial point of care. Current testing, including cytotoxicity and immunoassays require hours to days to complete, a time frame where treatment delay could extend disease complications. Even small improvements in the speed of diagnosis of treatable infectious disease could have major impacts on all hospital and nursing home populations but would be especially important in low-income or remote areas. The inventors as disclosed herein used C. difficile as a model organism (a non-infectious strain) to test the microfluidic device of the present invention. Naturally, our results are applicable for diagnosis of any bacterial, viral, or parasite presence in a biological sample.

The biological sample as used in the present invention can be any material that either contains or is suspected to contain cells. The sample may be blood, serum, sputum, saliva, urine, stool, bone marrow, consumable food/drink stuff, soil, water, or any other material that can be either directly added to the channel of the microfluidic device as disclosed herein or mixed with a small amount of buffer reagent to make the sample liquid enough to enter the channel.

The device of the present invention can be adapted to diagnose one or more, preferably multiple disease causing agents. For example, the microfluidic platform of the present invention allows one to create rapid, disposable, and inexpensive testing system for multiple infectious diseases.

The inventors have fabricated microfluidic devices as described, supra, that lyse cell. For example the microfluidic devices as disclosed herein can lyse microorganisms, for example bacteria cells, as well as viruses, pathogens and mammalian cells. In particular, the inventors have fabricated microfluidic devices as disclosed herein that can lyse gram-negative and gram-positive bacteria.

Examples of such microorganisms that can be lysed by the cell lysis device as disclosed herein is, for example a pathogens. Pathogens are microorganisms that potentially lead to infections and infectious diseases.

In some embodiments, the cell lysis device as disclosed herein can be used to lyse bacteria cells. In some embodiments, the bacteria are pathogens that lead to infection. Bacteremia refers to the presence of bacteria in the bloodstream, and where there are too many bacteria to be removed easily sepsis develops, causing severe symptoms. In some cases, sepsis leads to a life-threatening condition called septic shock. Bacilli are a type of bacteria classified according to their distinctive rod-like shape. Bacteria are either spherical (coccal), rod-like (bacillary), or spiral/helical (spirochetal) in shape. Gram-positive or gram-negative bacilli are distinguished Examples of gram-positive bacillary infections are are erysipelothricosis (caused by Erysipelothrix rhusiopathiae), listeriosis (caused by Listeria monocytogenes), and anthrax (caused by Bacillus anthracis). Within anthrax, pulmonary anthrax, gastrointestinal anthrax and anthrax skin sores can be distinguished. Examples of gram-negative bacillary infections are Hemophilus infections, Hemophilus influenzas infections, Hemophilus ducreyi (causes chancroid), Brucellosis (undulant, Malta, Mediterranean, or Gibraltar fever, caused by Brucella bacteria), tularemia (rabbit fever, deer fly fever, caused by Francisella tularensis), plague (black death, caused by Yersinia pestis, bubonic plaque, pneumonic plague, septicemic plague and pestis minor are distinguished), cat-scratch disease (caused by the bacterium Bartonella henselae), Pseudomonas infections (especially Pseudomonas aeruginosa), infections of the gastrointestinal tract or blood caused by Campylobacter bacteria (e.g. Campylobacter wlori [Helicobacter pylori]), cholera (infection of the small intestine caused by Vibrio cholerae), infections with other Vibrio spp., Enterobacteriaceae infections (cause e.g. infections of the gastrointestinal tract, members of the group are Salmonella, Shigella, Escherichia, Klebsiella, Enterobacter, Serratia, Proteus, Morganella, Providencia, and Yersinia), Klebsella pneumonia infections (severe lung infection), typhoid fever (caused by Salmonella typhi), nontyphoidal Salmonella infections, or Shigellosis (bacillary dysentery, an intestinal infection caused by Shigella bacteria). Bacteria that have a spherical shape are called cocci. Cocci that can cause infection in humans include staphylococci, streptococci (group A streptococci, group B streptococci, groups C and G streptococci, group D streptococci and enterocooci), pneumococci (cause e.g pneumonia, thoracic empyema, bacterial meningitis, bacteremia, pneumococcal endocarditis, peritonitis, pneumococcal arthritis or otitis media), and meningococci. Toxic shock syndrome is an infection usually caused by staphylococci, which may rapidly worsen to severe, untreatable shock.

In some embodiments, bacteria are Meningococci (Neisseria meningitidis), which can cause infection of the layers covering the brain and spinal cord (meningitis). Neisseria gonorrhoeae cause gonorrhea, a sexually transmitted disease. Spirochetal Infections are infections with spirochetes, corkscrew-shaped bacteria. Examples include infections with Treponema, Borrelia, Leptospira, and Spirillum. Treponematoses (e.g. yaws, pinta) are caused by a spirochete that is indistinguishable from Treponema pallidum (causes syphilis). Relapsing fever (tick fever, recurrent fever, or famine fever) is a disease caused by several strains of Borrelia bacteria.

In further embodiments, the device as disclosed herein is used for cell lysis of C. Difficile.

In another embodiment, the device as disclosed herein is used for cell lysis of other pathogens, for example but not limited to such a pathogen is Lyme disease (transmitted by deer ticks) is caused by the spirochete Borrelia burgdorferi. Other examples for infections with spirochetes are Leptospirosis (a group of infections including Weil's syndrome, infectious (spirochetal) jaundice, and canicola fever), or rat-bite fever).

In further embodiments, the device as disclosed herein is used for cell lysis of disease-causing anaerobic bacteria include clostridia, peptococci, and peptostreptococci. Other examples are Bacteroides fragilis, Prevotella melaminogenica and Fusobacterium. Infections with anaerobic bacteria include dental abscesses, jawbone infections, periodontal disease, chronic sinusitis and middle ear infection, and abscesses in the brain, spinal cord, lung, abdominal cavity, liver, uterus, genitals, skin, and blood vessels. Examples for Clostridial infections tetanus (lockjaw, caused by the bacterium Clostridium tetani), or Actinomycosis (a chronic infection caused mainly by Actinomyces israelii).

In yet further embodiments, the device as disclosed herein is used for cell lysis of Mycobacteria which causes Tuberculosis and leprosy, in particular by the airborne pathogen Mycobacterium tuberculosis, M. bovis, or M. africanum. Leprosy (Hansen's disease) is caused by the bacterium Mycobacterium leprae. Rickettsial infections are also known. Examples of diseases caused by Rickettsiae or Ehrlichieae are murine typhus (caused by Rickettsia typhi), Rocky Mountain spotted fever (caused by Rickettsia rickettsii), epidemic typhus (Rickettsia prowazekii), scrub typhus (Rickettsia-62 tsutsugamushi), Ehrlichiosis (Ehrlichia cants or closely related species), Rickettsial-pox, (Rickettsia akari), Q fever (Coxiella burnetii), or trench fever (Bartonella quintana).

Infections of the skin and underlying tissue are due to pathogens, for example, cellulitis, necrotizing fasciitis, skin gangrene, Iymphadenitis, acute Iymphangitis, impetigo, skin abscesses, folliculitis, boils (furuncles), erysipelas, carbuncles (clusters of boils and skin abscesses), staphylococcal scalded skin syndrome, erythrasma or paronychia (can be caused by many bacteria and fungi). Most of these are bacterial infections. The most common bacterial skin infections are caused by Staphylococcus and Streptococcus. Also encompassed is microorganism that cause skin infections via, for example fungi, for example ringworm, Athlete's foot (foot ringworm, caused by either Trichophyton or Epidermophyton), jock itch (groin ringworm), scalp ringworm, caused by Trichophyton or Microsporum), nail ringworm and body ringworm (caused by Trichophyton). Candidiasis (yeast infection, moniliasis) is an infection by the yeast Candida. The following types of candida infections can be distinguished: Infections in skinfolds (intertriginous infections), vaginal and penile candida infections (vulvovaginitis), thrush, Perleche (candida infection at the corners of the mouth), candidal paronychia (candida growing in the nail beds, produces painful swelling and pus). Candida can also lead to generalized systemic infections especially in the immunocompromised host. Tinea versicolor is a fungal infection that causes white to light brown patches on the skin. The skin can also be affected by parasites, mainly tiny insects or worms. Examples are scabies (mite infestation), lice infestation (pediculosis, head lice and pubic lice are two different species), or creeping eruption (cutaneous larva migrans, a hookworm infection). Many types of viruses invade the skin. Examples are papillomavirusses (causing warts), herpes simplex virus (causing e.g. cold sores), or members of the poxvirus family (molluscum contagiosum (infection of the skin, causing skin-colored, smooth, waxy bumps).

In further embodiments, the device as disclosed herein is used for cell lysis of cells comprising a parasite, such as a single-celled animal (protozoan) or worm, that survives by living inside another, usually much larger, organism. Examples for parasitic infections are—Amebiasis (caused by Entamoeba histolytica), Giardiasis (Giardia lamblia), Malaria (Plasmodium), Toxoplasmosis (Toxoplasma gondii), Babesiosis (Babesia parasites), Trichuriasis (Trichuris trichiura, an intestinal roundworm), Ascariasis (Ascaris lumbricoides), Hookworm Infection (Ancylostoma duodenale or Necator americanus), Trichinosis (Trichinella spiralis), Toxocariasis (visceral larva migrans, caused by the invasion of organs by roundworm larvae, such as Toxocara canis and Toxocara cati)), Pork tapeworm infection (Taenia solium), or Fish tapeworm infection (Diphyllobothrium latum).

In further embodiments, the device as disclosed herein is used for cell lysis of cells infected by fungi. Fungi tend to cause infections in people with a compromised immune system. Examples for fungal infections are Histoplasmosis (caused by Histoplasma capsulatum), Coccidioidomycosis (Coccidioides immitis), Blastomycosis (Blastomyces dermatitidis), Candidiasis (caused by strains of Candida, especially Candida albicans), or Sporotrichosis (Sporothrix schenckii).

In further embodiments, the device as disclosed herein is used for cell lysis of cells infected by viruses. Non-limiting examples of viral infections are as follows; respiratory viral infections are, for example, common cold (caused by Picornaviruses [e.g. rhinoviruses], Influenza viruses or respiratory syncytial viruses), Influenza (caused by influenza A or influenza B virus), Herpesvirus Infections (herpes simplex, herpes zoster, Epstein-Ban virus, cytomegalovirus, herpesvirus 6, human herpesvirus 7, or herpesvirus 8 (cause of Kaposi's sarcoma in people with AIDS), central nervous system viral infections (e.g. Rabies, Creutzfeldt-Jakob disease (subacute spongiform encephalopathy), progressive multifocal leukoencephalopathy (rare manifestation of polyomavirus infection of the brain caused by the JC virus), Tropical spastic paraparesis (HTLV-I), Arbovirus infections (e.g. Arbovirus encephalitis, yellow fever, or dengue fever), Arenavirus Infections (e.g Lymphocytic choriomeningitis), hemorrhagic fevers (e.g. Bolivian and Argentinean hemorrhagic fever and Lassa fever, Hantavirus infection, Ebola and Marburg viruses).

One example of a common virus is Human immunodeficiency virus (HIV) infection is an infection caused by HIV-1 or HIV-II virus, which results in progressive destruction of Lymphocytes. This leads to acquired immunodefciency syndrome (AIDS). Other viruses include for example Hepatitis A, hepatitis B, hepatitis C, SARS, avian flu etc.

Other pathogen viruses include sexually transmitted (venereal) diseases, for example syphilis (caused by Treponema pallidum), gonorrhea (Neisseria gonorrhoeae), ehaneroid (Hemophilus duereyi), lymphogranuloma venereum (Chlamydia trachomatis), granuloma inguinale (Calymmatobaeterium granulomatis), nongonoeoeeal urethritis and ehlamydial eervieitis (caused by Chlamydia trachomatis, Ureaplasma urealytieum, Trichomonas vaginalis or herpes simplex virus), triehomoniasis (Trichomonas vaginalis), genital candidiasis, genital herpes, genital warts (caused by papillomaviruses), or HIV infection.

In another embodiment, the pathogen is an infection with opportunistic pathogens, often infecting people with impaired immune system, such as for example but are not limited to nocardiosis (caused by Nocardia asteroides), aspergillosis, mucormyeosis, and eytomegalovirus infection.

The inventors have fabricated a method that is completely scalable, with a microfabrication design that is applicable to lysis of a wide variety of cells in unprocessed non-treated biological samples. Such a microfabrication design of the microfluidinic devices as disclosed herein comprise materials and processes used in mass production.

Lysing bacterial cells in the microfluidic platform has posed a significant challenge in the art. While mammalian cells can be lysed by a combination of lysis buffer and simple mixing, lysing bacteria cells takes significantly more effort due to the nature of the cell wall. The inventors have demonstrated herein that mechanical shear induced by flow disruption, and in some instances, optionally with the addition to mixing with a lysis buffer can break apart bacteria, such as C. Difficile.

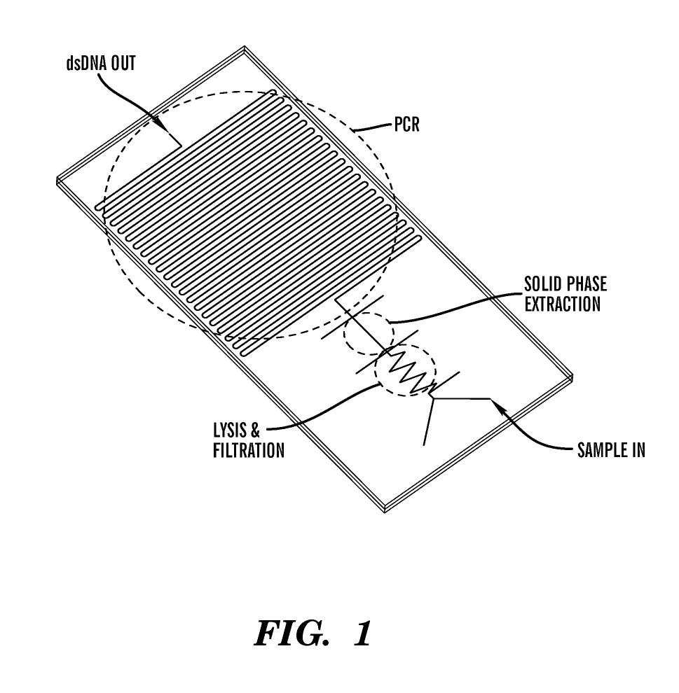

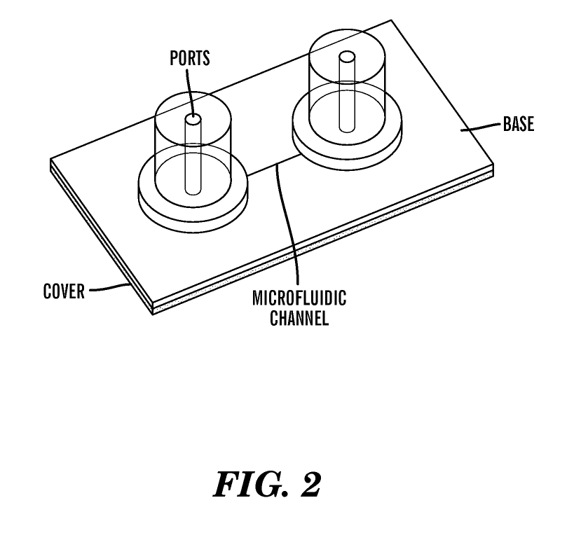

The modified microfluidic mixing channels as described herein, and shown for example in FIG. 1 shows a sample preparation devices comprising a for cell lysis module and an optional biomolecule extraction module, for example a nucleic acids extraction module, and a module for biomolecule analysis, for example PCR module. In such an embodiment, biological samples from a patient can be completely processed using a single device and diagnosis performed at the point of care.

The cell lysis device as disclosed herein significantly improves diagnostic of infections, for example, diagnostic bacterial infections or bacterial cells present in a biological samples from a patient, enabling rapid point-of-care diagnostics to be performed. While such point of care methods often require on extraction/purification of biomolecules from a cell present in a biological sample, an essential step prior to this in molecular diagnostics that use nucleic acid probes is cell lysis of human pathogens. The methods as disclosed herein enable lysis of cells, for example microorganisms, bacteria, plant cells and pathogens, using a microfluidic device as disclosed herein, which can be optionally combined with microfluidic devices for extraction/purification of biomolecules for point of care diagnostics. Such microfluidic devices also may comprise a real time PCR step, enabling fast, highly specific detection of microorganisms in a biological sample from a subject or patient. Sample and reagent consumption will be greatly reduced. In some embodiments, all processes will be carried out on a single chip with little sample pretreatment, significantly reducing processing time and minimizing the potential for cross contamination. The plastic chips are easily prototyped for rapid testing of new layouts. The devices are inexpensive and disposable.

The method disclosed herein permits immobilizing carbon particles in a porous polymer monolith to form a microscale on-chip cell-lysis system. The monolithic column is formed by in situ UV polymerization of a monomer mixture impregnated with silica particles. The porous polymer monolith comprising carbon particles is covalently attached to the walls of the microchannels to prevent its displacement when samples are flowed through the channels. The inventors have demonstrated the ability of these monoliths to lyse both gram-positive and gram-negative bacteria from simulated biological samples.

In addition to mechanical force and obstacles to lyse the cells, one may also use a chatotrophic buffer.

It will be understood by one skilled in the relevant arts that not all carbon particles will be suitable with all polymer monoliths. For example, carbon nanotubes may be more useful some embodiments than carbon graphine. However, it would not cause a skilled person to undertake undue experimentation to learn that using monomers and solvent conditions that are more hydrophilic can increase the number of embedded carbon particles to the desired about. Embodiments of the present invention will now be described by way of the Examples. It will be understood that the scope of the present invention, and the methods and devices as disclosed herein by specific embodiments exemplified herein.

### EXAMPLES

The examples presented herein relate to a method of bacterial lysis. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Pre-Polymerformations. Protocol for Making Zeonex 690R Wire-Imprinted Microchannels

Production of the cover plate. 1) Clean Stainless Steel press plates using first Acetone, (clean with Kim wipes if necessary), second methanol, and third isopropenol. Place clean plates on sheet metal “clean table”. 2) Heat up hot press, and 3) set temperature to 351° C., (Zeonox 690R specific). 4) Place 1″ circle of Zeonex beads with a depth of about 2 layers in the center of one of the press plates sitting on the “clean table”. 5) Once the press has reached 351±1° C., carefully place press plate with Zeonex on the Platen 2. 6) Place the second press plate on top of the pile of Zeonex beads as centered as possible, being careful to minimally disturb them. 7) Bring Platen 1 and Platen 2 together until the top press plate is in contact with Platen 1. 8) If the temperature of the top press plate drops significantly (more than 5° C.), wait until temperature rises to within 351±1° C. again. 9) Once temperature is stable around 351° C., press plates to 1000±10% psi. Maintain 1000 psi for the full five minutes. 10) After the five minute duration remove the press plates from the press and place on the “clean table”. Allow the press plates to cool for 2 minutes before separating.

11) After 2 minutes separate plates and carefully pry loose Zeonex disk with tweezers, shims or bending the press plates. (In some instances, if sticking occurs, immediately after removing plates from hot press, run cold water over both sides of the plate to facilitate rapid cooling). 12) Rinse both sides of Zeonex disk with methanol then isoproponol and drip dry in hood. 13) Cover plate is completed.

Production of the base plate with microchannels. 14) Use razor blade to scrape press plates if Zeonex sticking occurs. Repeat steps 4-8 above. 16) Once temperature is stable around 351° C., press plates to 250±10% psi, 250 psi for the full five minutes. 17) repeat steps 10-11 above. 18) Reduce press temperature to 315° C. 19) Place wires on base plate in desired pattern on the smoother side of the plate as it will be used for bonding. 20) Once the press has reached 315±1° C., carefully place press plate with Zeonex disk/wires on the Platen 2. 21) Place the second press plate on top of the Zeonex disk as centered as possible, being careful to minimally disturb the wire arrangements on the disk. 22) Bring Platen 1 and Platen 2 together until the top press plate is in contact with Platen 1. 23) If the temperature of the top press plate drops significantly (more than 5° C.), wait until temperature rises to within 315±1° C. again. 24) Once temperature is stable around 315° C., start timer and press plates to 250±10% psi and maintain 250 psi for the full 1 min 40 s. 25) After the 1 min 40 seconds duration remove the press plates from the press and place on the “clean table”. Allow the press plates to cool for 1 minute before separating.

26) After 1 minute separate plates and carefully remove the Zeonex disk using tweezers, shims or bending the press plates. 27) Remove the wires by gently bending the disk to get the wires to pop out, creating the channels and then prying them out with tweezers. 28) Use drill with 1/16^{th} inch drill bit to drill entrance and exit ports on bottom plate, ensuring the exit ports are on the channel imprinted side, and clean out the channels to remove shavings created by drilling. 29) Rinse both sides of the Zeonex disk with methanol then isoproponol. Let drip dry in hood. 30) Base plate is completed

Bond the base plate with channels with the cover plate. 31) Reduce press temperature to 276° C. 32) Place cleaned cover plate and base plate on the press plate, with the smoother surface of the cover plate facing the base plate, and the base plate with the imprinted channels facing the cover plate. 33) Place top press plate on top of cover and base plates and place assembly in hot press. 34) Bring Platen 1 and Platen 2 together until the top press plate contacts Platen. 35) Allow temperatures to stabilize at 276° C. 36) Once temperatures are stable at 276° C., start timer and press plates to 1000±10% psi, and maintain 1000 psi for 1 min 45 s. Increase pressure to 2000 psi for the last 15 seconds. 37) After the 2 minute duration remove the press plates from the press and place on the “clean table”. Allow the press plates to cool for 1 minute before separating. 38) After 1 minute separate plates and carefully remove Zeonex disk. 39) Repeat steps 31 to 38 as many times as necessary to get a good bond between the base plate and cover plate, which can be done for 3 to 6 times, changing orientation and flipping disk each time.

One can also formulate multiple substrates, comprising the base plate and cover with microfluidic channel by slight modification of the above protocol by someone of ordinary skill in the art.

Testing Protocol: Quantification of Extracted dsDNA from Bacteria Run Through a MWNT Packed Porous Polymer Monolith in a Microfluidic Channel

1) Epoxy coned ports from Upchurch Scientific N-333 Nanoport assemblies to the inlet and outlet ports of the channel to be tested with JB Weld epoxy. Use of the Nanoport gasket is not required. Ensure thorough coverage of the base of the Nanoport outside of the gasket groove. Utilize the gasket groove as a sort of epoxy reservoir to protect the chip inlet port from being clogged with epoxy. Utilize binder clips to provide pressure and hold ports in place. Allow epoxied ports to dry overnight.

Culture 5 ml of bacteria for between 14 and 24 hours in appropriate media, and measure the Optical Density (OD) @ 600 nm and determine the mean dilution ratio to ensure that the calculated OD will be close to, but not exceed 0.3, (i.e. if the measured value is 1.5, the minimum dilution ratio will be 4:1. Dilute the remainder of the culture in accordance with the dilution ratio, (i.e. if the OD value measured was 1.5 the minimum dilution ratio is 4:1 and 4 parts media should be mixed with 1 part liquid culture. Separate out 1.2 ml of the diluted culture into an eppendorf tube for use as the positive control. Follow Qiagen Blood and Cell Culture Mini Kit Protocols for Bacteria Lysis and DNA Extraction to prepare positive control in parallel with testing. 9) Centrifuge remainder of culture at 14000×g rcf for 10 minutes. Aspirate media and discard and resuspend the pellet in an equivalent volume of 0.85% NaCl/(RNAse and DNAse free filtered H2O), or Qiagen Buffer B1 to create the bulk test sample. Set aside 1 ml of the test sample for a Colony Forming Unit (CFU) count and as a negative control. Add 45 ul/ml Proteinase K to the bulk test sample. Attach 1/16th″ capillary tubing to a clean syringe capable of holding at least 3 ml of volume by using Upchurch Scientific P-659 and MicroTight 10-32 coned fitting. 15) Load the syringe with the remainder of the bulk test sample (approximately 3 ml). Deaerate the syringe by inverting and pushing the plunger until the bulk test sample begins to come out of the capillary tubing. Set the syringe pump to between 250 and 500 ul/hr, with the desired volume to 100 μl at a minimum. Load the syringe on the syringe pump. Attach the capillary tubing to channel inlet port with the 10-32 nut and ferrule that came with the N-333 Nanoport assay. Attach a length of 1/16th″ capillary tubing to the outlet port and place free end of the capillary tubing in an eppendorf tube. Active syringe pump and allow a minimum of 100 ul of the bulk test sample to pass through the chip. If testing multiple channels, detach syringe pump at channel Nanoport and attach to second, third, fourth, etc. test channel. If using capillary tubing on the channel output, clean the tubing with 70% EtOH and then Millipore H20 before using for the next channel.

For a positive control, use Qiagen Protocol for efficient test completion. Also complete the Colony Forming Unit (CFU) protocol to prepare the CFU plate for incubation: Take 100 ul from each tested sample or the negative control and load a disposable syringe equipped with a filter capable of a 0.2 micron filtration and a minimum volume of 500 ul or less. Add 400 ul of additional 0.85% NaCl or buffer B1, (whichever the cells were suspended in for testing), to the syringe prior to filtering. Deaerate by inverting the syringe, removing the filter, utilizing the plunger to force the sample to the top of the syringe without spilling. 26) Reattach the filter. 27) Push with reasonable force on the plunger to filter the test sample, (no greater than ˜1.0 lbs maximum. Collect the filtered sample in a separate clean eppendorf. 28) Repeat steps 22-27 as many times as necessary to filter samples from each tested channel.

Follow Novagen Pellet Paint Protocol to isolate and purify the DsDNA from each tested channel. Once the pellet paint protocol is complete for each tested sample, resuspend the resultant nucleic acid pellet in 100 ul of 1×TE, Ph 8.0. Finish any of the remaining

Extracted nucleic acids from the original 1.2 ml sample should re-suspended in 1.0 ml of 1×TE, Ph 8.0), Make Quant-it Picogreen according to the Quant-it Picogreen dilution ratio (200:1) in 1×TE with, Ph 8.0. Pipet 50 ul of each purified nucleic acid mixture from each test sample, positive control and negative control 3× into a black/Clear bottomed Costar 96 well plate. Pipet 50 ul of the Picogreen/TE mixture into each of the wells where sample resides. Use the microplate reader to measure fluorescence of each tested sample by exciting at 488 nm and emitting at 525 nm. Compare the fluorescence values with the applicable low or high range standard curve generated by following the Picogreen standard curve generation protocol to determine the concentration of DsDNA extracted from each sample.

Experimental Methods and Apparatus

Experimental methods were developed to evaluate device performance. The chosen approach was to evaluate the concentration of dsDNA that could be extracted by using the microfluidic device given an input cell concentration and comparing it to both negative and positive controls to determine the relative effectiveness of the device.

Preparation of Positive Control A. Qiagen Blood and Cell Culture Mini-Kit and Qiagen Genomic DNA Buffer Set were purchased from Qiagen Inc, of Valencia, Calif. The Qiagen kit represents a standard approach to the isolation, extraction and purification of dsDNA and serves as an excellent positive control. The Qiagen protocol includes many manually executed steps in order to extract purified dsDNA from a sample culture of bacteria. First, an optical density measurement is taken of the culture at a wavelength of 600 nm to determine approximate cell concentration within the sample. The bacterial culture is then diluted in its growth media to an acceptable concentration as defined by the Qiagen protocol to avoid clogging of the Genomic tips provided with the kit. Secondly, the bacteria is centrifuged and re-suspended in a detergent and TE based Qiagen lysis buffer where RNAse, Proteinase K and freshly prepared lysozyme are added to affect lysis. The sample is then mixed and incubated for at least thirty minutes to allow lysis to occur. Following lysis, another Qiagen buffer is added to denature any remaining DNA binding proteins in conjunction with remaining Proteinase K. Third, the sample is loaded into a Qiagen Genomic Tip, (already equilibrated with another detergent based buffer), and allowed to flow through into a waste receptacle by gravity. During this process the dsDNA binds to the Genomic tip filter. Fourth, after a wash step to remove contaminants, the dsDNA is eluted into a clean container with an elution buffer. Finally, the dsDNA is precipitated by the addition of isopropanol and centrifuging, followed by a 70% ethanol wash and re-suspended in 1×TE at a Ph of 8.0. The final product is a suspension of dsDNA in TE.

Fluorescence Based Detection of dsDNA Concentrations. In order to detect the concentration of dsDNA extracted by both the positive control and test samples, (inc. negative control), fluorescence-based detection was chosen. The fluorescence based detection method is accomplished by adding a fluorescent dye to the dsDNA suspension in TE and measuring resultant fluorescence at a particular wavelength when excited with light at a different wavelength. The equipment used to perform these measurements was a Molecular Device Spectramax M5 Microplate Reader. The suspensions of dsDNA were pipetted into Costar Black walled/clear bottomed 96 well plates and then fluorescence was measured with the Spectramax M5 using an endpoint protocol. The fluorescent dye chosen for dsDNA quantification was Invitrogen's Quant-it Picogreen. Early experimentation was also conducted using Hoechst 33258; although Picogreen was ultimately used exclusively due to its improved sensitivity, specificity to dsDNA, and universal concentration, (one concentration in TE could be used to detect many orders of magnitude different concentrations of dsDNA, as opposed to Hoescht 33258, which required different dye concentrations to detect different dsDNA concentrations)

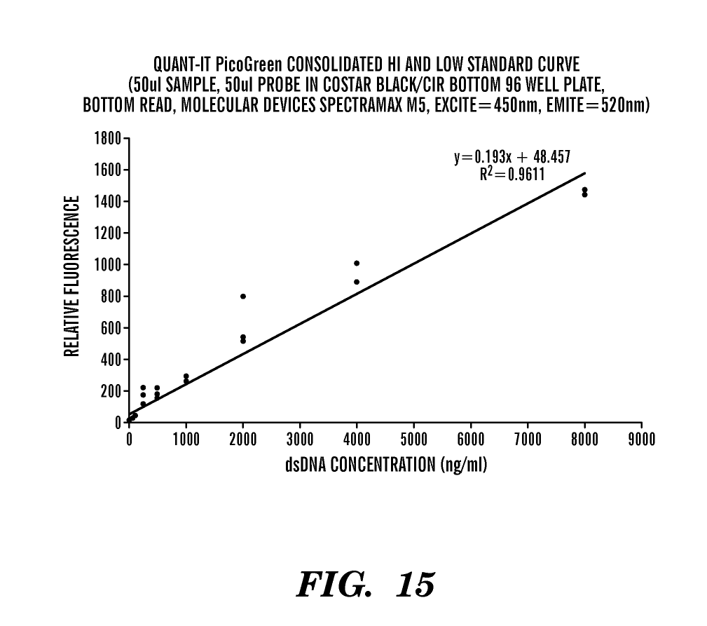

In order to correlate the relative fluorescence units to dsDNA concentrations standard curves were generated at each emission and excitation wavelength used with lambda-phage dsDNA purchased from Sigma Aldrich in accordance with the Picogreen Standard curve protocols, as shown in see FIG. 15.

Two different excitation and emission wavelengths were used throughout testing. 480 nm exitation/520 nm emission, (Novagen specification), and 488 nm exitation/525 nm emission, (determined to produce larger fluorescence signals).

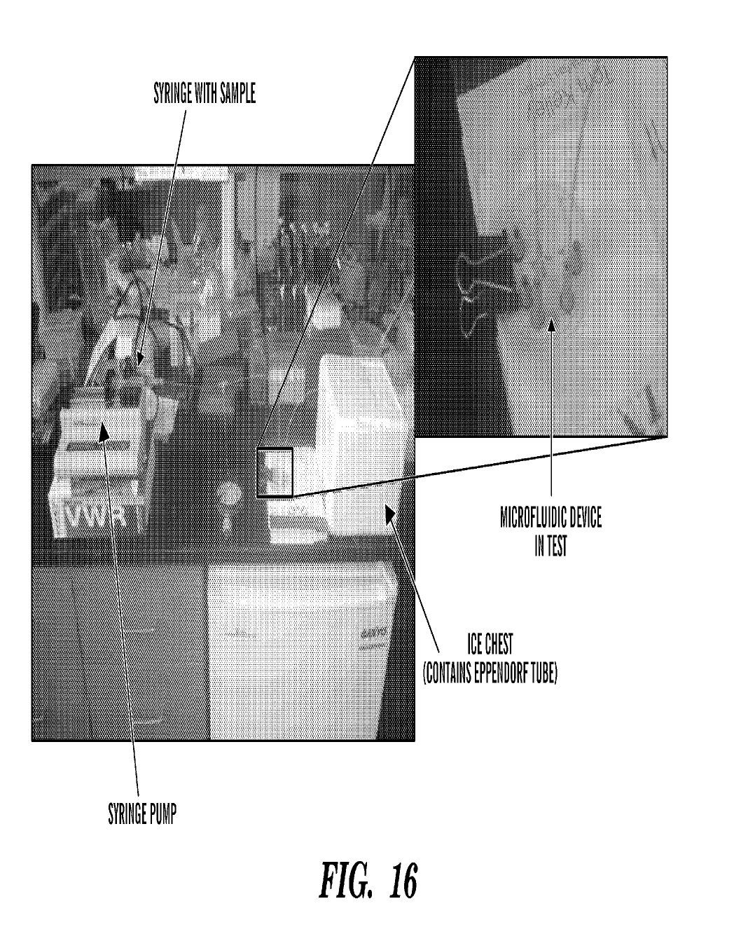

Experimental Apparatus. The experimental apparatus consisted of a KD Scientific KDS100 syringe pump, Becton Dickenson 10 CC plastic syringe, Upchurch Scientific 1/16″ PEEK capillary tubing and Upchurch Scientific Luer to 1/16″ capillary tubing fitting. The capillary tubing connected the syringe to the port on the microfluidic chip and during some tests to the eppendorf microtube being used to collect the device output. In other instances only a single Nanoport was utilized and device output was captured using a pipetter. A pressure gauge was used to monitor backpressure. A picture of the testing apparatus is shown in FIG. 16.

Experimental Procedure. In order to test the microfluidic devices the liquid cell culture of the chosen bacteria is first diluted to a cell concentration level acceptable for use with the positive control protocol. The diluted culture is then broken into a positive control sample, test sample(s) and negative control sample(s). The positive control sample proceeds on in accordance with the Qiagen Sample Preparation and Lysis Protocol for Bacteria and Genomic-tip Protocols as previously described.

The test samples and negative controls are then centrifuged and re-suspended in the testing media. Re-suspension is required because of the extremely high protein concentrations in the bacterial growth media. Despite the fact that Quantit Picogreen is extremely specific to dsDNA, it does fluoresce in the presence of proteins to a much lesser degree. With the amount of protein present in the media it became evident that the protein binding related fluorescence would overwhelm that generated by dsDNA binding. Two testing mediums were used during experimentation. The first media was 0.85% sodium chloride in purified water. The second media used was Qiagen Buffer B1, a TE based buffer including small concentrations of Tween-20 and Triton X-100 detergents. Proteinase K, in an identical volumetric ratio to that utilized in the Qiagen protocol, is then added to digest any left over media proteins. Once the experimental apparatus is in place the test samples are loaded into the syringe and the syringe is deaerated. The connections are made to the microfluidic channel to be tested and the testing begins. The syringe pump is set to a flow rate varying between 250 ul/hr and 1000 ul/hr depending on the device being tested and the intent of the test. The syringe pump forces the sample through the microfluidic channel, where it is collected on the other side via pipetter or capillary tube feed to an eppendorf microtube.

Typically 100 ul of sample is run through each channel for each test. Once at least 100 ul has been processed through the device, 100 ul of it is filtered using a 4 mm Nalgene 0.2 micron syringe filter attached to an Exel 3 cc disposable syringe, (both purchased from Fischer Scientific). Before filtration an additional 400 ul of whichever media it was run through the device suspended in is added to the syringe to ensure the minimum filtration volume is met. This step is intended to remove all cell debris, un-lysed cells and any other channel debris that could contaminate the sample and be detrimental to downstream processing or contribute to fluorescence and thus false positives. This of particular importance because Quant-it Picogreen is a cell-permeant dye that will cause fluorescence in un-lysed cells. Once the sample is filtered, the dsDNA is precipitated out with the Pellet Paint Co-Precipitant, purchased from VWR International. The Pellet Paint Co-Precipitant is a bright pink dye that binds to nucleic acids and allows as little as 2 ng/ml of nucleic acids to be visable during precipitation. The precipitation itself is conducted through the use of ethanol and 70% ethanol in RNAse free water washes and repeated aspiration of the precipitants from the microtube in accordance with the Novagen protocol.

Finally, the precipitated dsDNA is re-suspended in 1×TE, pH 8.0. The suspension of dsDNA and TE is then quantified using fluorescence detection methods as discussed herein. The negative control goes directly from the microtube in which it is re-suspended following removal of the bacterial growth media into the filtration step. It never comes in contact with the syringe or channel and serves as a comparison with the samples tested in then channels to see the contribution provided by the channel to the final dsDNA concentration extracted.

### Example 1

### Surface Treatment of Carbon Nanotubes Prior to Embedding in Monolith

Multi-Walled Carbon Nanotubes within Porous Polymer Monoliths. After assessing and testing a variety of pre-polymer systems for use in the device, the inventors discovered that a pre-polymer system comprising the non-polar solvents, (cyclohexanol/dodecanol) for use with the non-polar monomer (BUMA) was selected. The polar solvents selected, (ethanol/methanol), were selected based upon their miscibility with the polar monomer, (GMA), and results reported in the literature⁴². The confidence in this pairing was high due to demonstrated successes within the laboratory⁴¹.

In some instances the inventors sometimes added additional constituent parts are added to the pre-polymer for functionality, such as 2-acrylamido-2-methyl-1-propane sulfonic acid, which is frequently used as an electro-osmotic flow promoter, (EOF). Details on each of the pre-polymer formulations that can be used are disclosed in the methods section entitled “detailed protocol for fabricating pre-polymer formulations”.

Following appropriate pre-polymer formation, the Inventors then focused on processing issues, such as for example combining the pre-polymer solution and carbon nanotubes prior to cross-linking. Initially, the multi-walled carbon nanotubes were measured by weight, added to one of the solvents, (cyclohexanol), in known quantities and subsequently mixed with the remainder of the BUMA based pre-polymer solution. It was found that the multi-walled carbon nanotubes were not particularly miscible within the nonpolar solvent BUMA pre-polymer solution. The multi-walled nanotubes tended to clump together and come out of suspension shortly after mixing.

Further research was conducted and conversations with Nanolabs, a manufacturer of Carbon Nanotube suspensions, confirmed that surface treatment along with ultrasonication of the nanotube/solvent mixture is required in order to facilitate effective and stable suspension. It was further revealed that even following surface treatment and ultrasonication the resultant suspension was only stable when a polar solvent was used.

The surface treatment of the carbon nanotubes was conducted both at Boston University, (treatment of nanotube powder), and Nanolabs, (treatment prior to suspension in ethanol. The methodology utilized in both instances was based upon research conducted by Shaffer et al. and consisted of performing a chemical oxidation via refluxing the carbon nanotubes in 1:3 Nitric and Sulfuric Acid⁴³. At Boston University this procedure consisted of refluxing at 140° C. for 1 hr. Following oxidation the carbon nanotubes were mix-cooled for 10 minutes. The carbon nanotubes were then extracted using a sintered glass filter and washed with purified water until a pH of ˜7 was achieved. The nanotubes were then vacuum dried and recollected in powder form. Carbon nanotube loss during refluxing was minimal.

The functionalized nanotubes were suspended in cyclohexanol and ultrasonicated for 30 minutes at a 50% duty cycle. The resultant suspension appeared to be more stable and exhibited less evidence of nanotube clumping than suspensions prepared prior to ultrasonication and functionalization. It is also noteworthy to mention that concerns do exist when ultrasonicating carbon nanotubes in an attempt to provide a uniform aqueous suspension. Research conducted by Lu et al. indicated that ultrasonication can result in damage to carbon nanotubes as well as carbon nanoparticles ultimately leading, (over extended sonication periods), to the formation of amorphous carbon⁴⁴. An experiment was conducted to attempt to qualitatively assess the impact of ultrasonicating the cyclohexanol/MWNT suspensions with concentrations ranging from 0.0025 to 0.25M for varying periods in an attempt to minimize the sonication period. The results indicated vary little difference in result between 30 and 60 minute sonication periods; therefore, 30 minute sonication periods were used throughout the rest of the processing conducted. Furthermore, post polymerization SEMs that were taken seem to suggest that significant mechanical damage of the carbon nanotubes used in this research did not occur.

### Example 2

### Generation of Polymers Containing Carbon Nanotubes

The inventors determined the concentrations of nanotubes to use as part of the overall pre-polymer system. The inventor assessed the concentrations of BUMA based pre-polymer solutions with nanotube concentrations from 0.001M to 0.5M that resulted in success fabrication. The inventors discovered that at the higher concentrations the repeatability began to suffer and after reviewing scanning electron micrographs a concentration of 0.25M was selected for repeated fabrication purposes.

In the case of the GMA based pre-polymer system the stock solution purchased from Nanolabs was initially used, (0.0033M in ethanol), providing a much lower concentration than that used in the BUMA system. After discovering a higher success with the lower concentrations, the inventors used suspension that were concentrated ten-fold and a larger concentration, (but still much lower concentration than used in the BUMA system) to fabricate the GMA porous polymer monoliths.

Overall, the inventor assessed two pre-polymer solutions for down-stream testing. Their Exact contents are listed in Table 1 below.

Table 1: Pre-Polymer Solutions Used in Fabrication of Porous Polymer Monoliths for Testing.

Pre-Polymer Solutions Tested

| | | Percentage of | | Percentage of |
|---|---|---|---|---|
| | Non-Polar | Content by | Polar | Content by |
| | System Parts | Volume | System Parts | Volume |
| Monomer | BUMA | 18% | GMA | 18% |
| Crosslinker | EDMA | 14% | EDMA | 14% |
| Photoinitiator | DMPAP | 1% | DMPAP | 1% |
| Solvent 1 | 2.27M Cyclohexanol | 10% | .033M Ethanol | 27% |
| | w/CNTS | | w/CNTS | |
| Solvent 2 | Cyclohexanol | 10% | Methanol | 40% |
| Solvent 3 | Dodecanol | 47% | — | — |

### Example 3

### Grafting Channels for Adherence of Carbon Nanotube Impregnated Porous Polymer

Processing of the Carbon Nanotube Impregnated Porous Polymer Monolith

Once a pre-polymer solution was prepared and a polymeric microfluidic chip was fabricated the pre-polymer solution was pipetted into the channels and in-situ polymerization can be used to create the porous polymer monolith. Before this can happen, the inventors added an additional grafting layer to the inside of the channels. Since Zeonex is a Teflon-like material, it exhibits extremely low surface energy making it difficult to get the porous polymer monolith to bind to the channel wall. In order to solve this problem, the inventors used a “grafting mix” comprising a pre-polymer solution, comprising of a 1:1 mixture of Ethlyene diacrylate, (EDA) and Methyl methacrylate, (MMA), combined with enzophenone (an photo-sensitizer), and introduced to the channels following a thorough methanol wash (demonstrated by Bhattacharrya and Klapperich⁴⁵).

This “grafting mix” is cross-linked to create a layer on the channel walls to which the porous polymer monolith can covalently attach. It is also noteworthy to mention that the grafting mix was also fabricated in then absence of EDA with similar results. The inventors used the same grafting layer to promote adhesion for both polar and nonpolar pre-polymer systems.

Following the grafting step, the inventors removed excess grafting solution from the channel and applied the pre-polymer solution. The pre-polymer solution is then cross linked in a UV cross-linker, pausing only to flip the chip over half of the way through. Cross linking parameters were done based on previously established parameters and variations of parameters by Bhattacharyya and Klapperich for the BUMA system⁴⁶. Following cross-linking, the resulting porous polymer monoliths were washed extensively with methanol to remove any resultant solvents and to ensure good pore formation.

Fabrication Results: Non-Polar (BUMA) Porous Polymer Monoliths.

The inventors were able to fabricate non-polar BUMA porous polymer monoliths in an easy and highly repeatable method, with the vast majority of those channels fabricated resulting in well formed porous polymer monoliths with pore sizes of approximately within the range of a few microns.

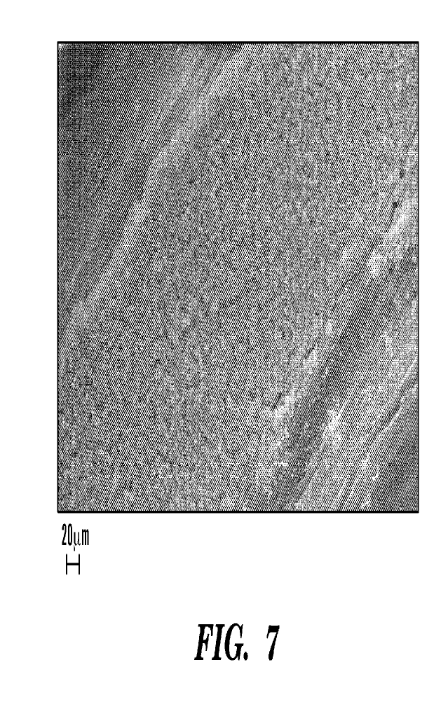

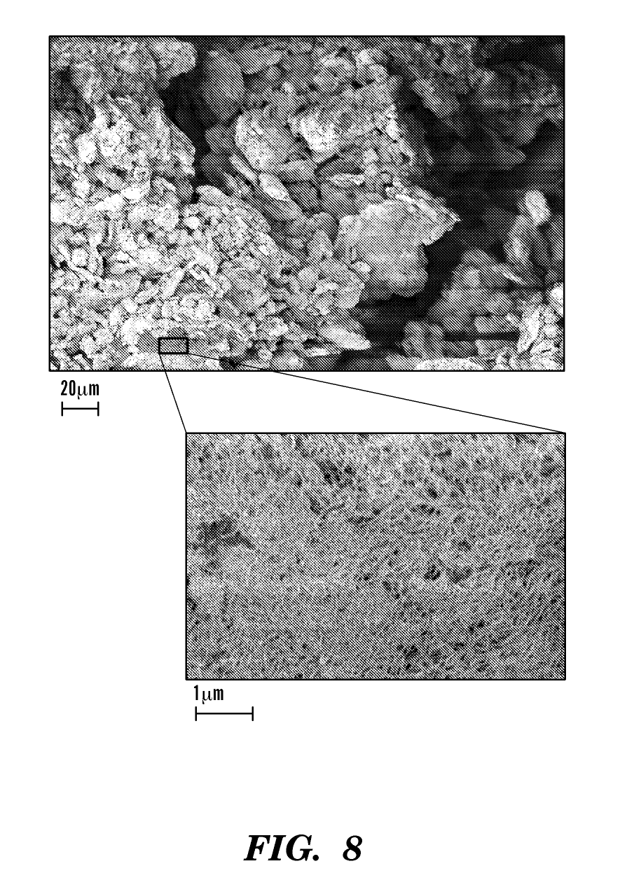

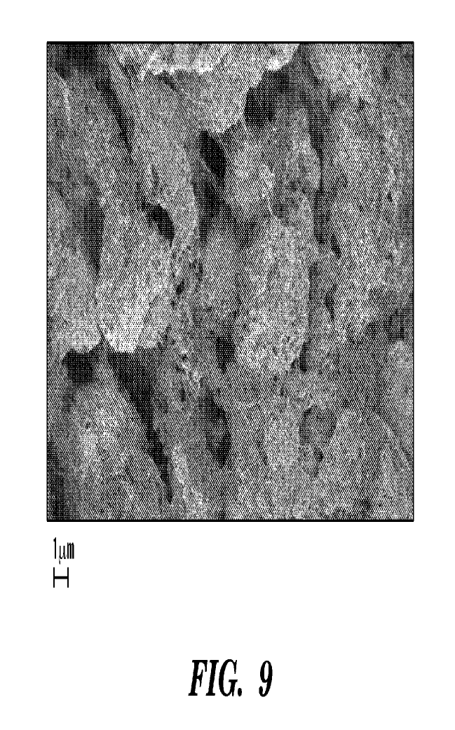

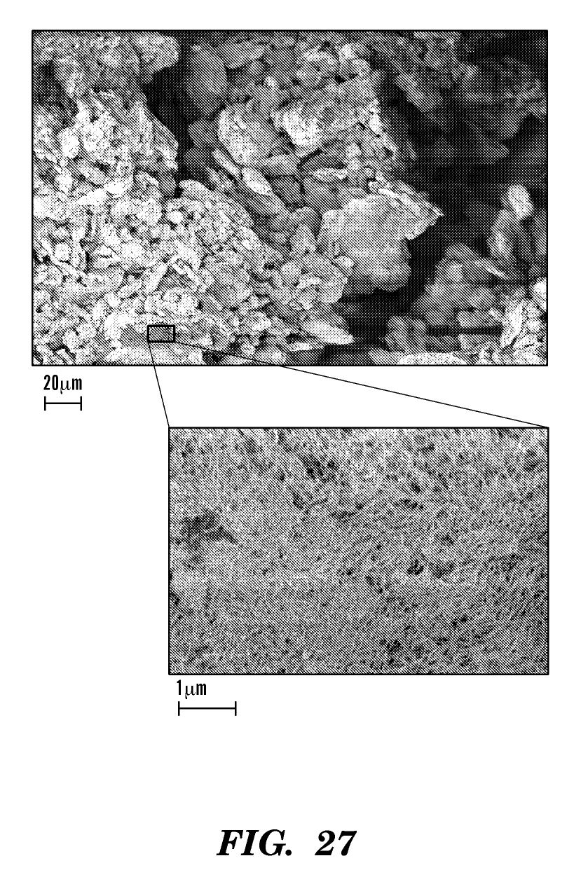

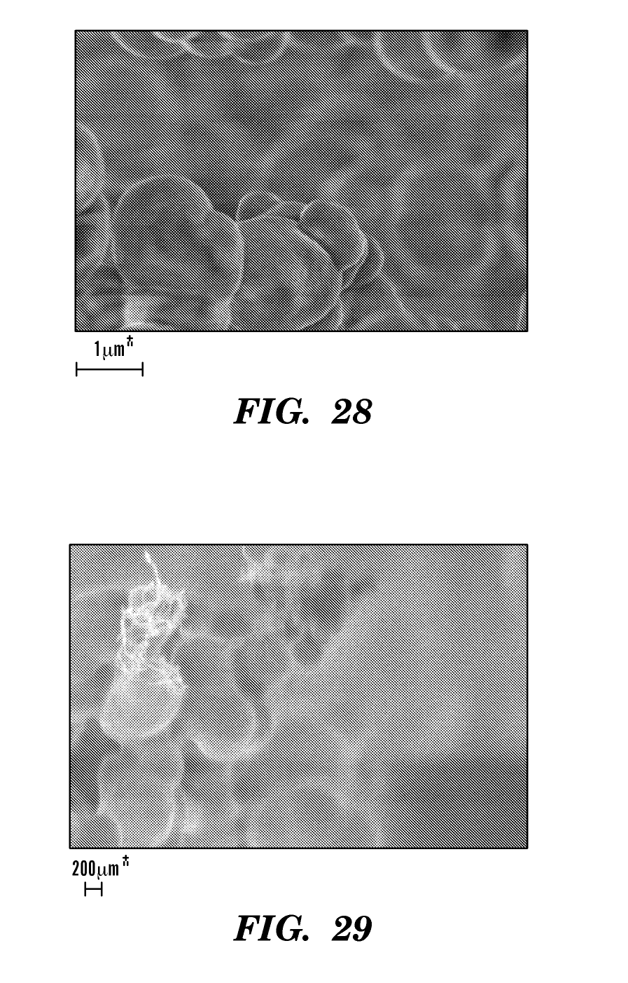

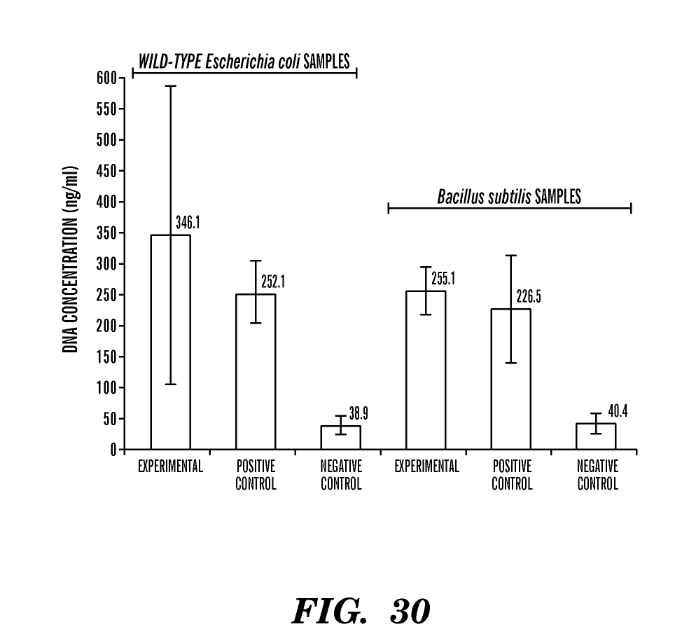

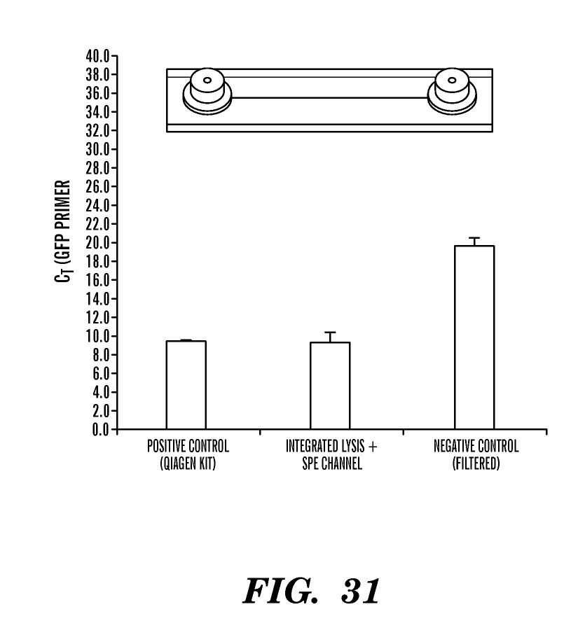

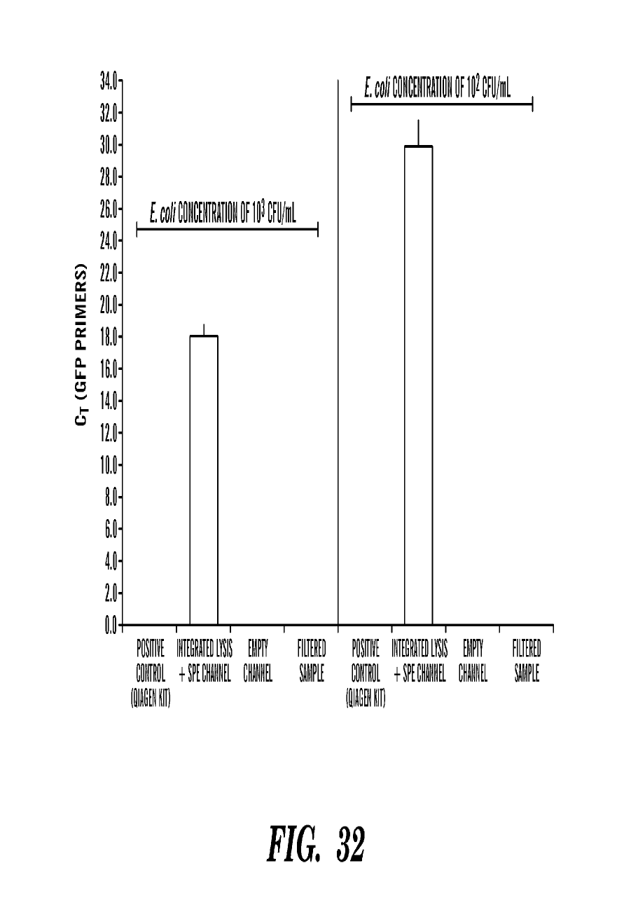

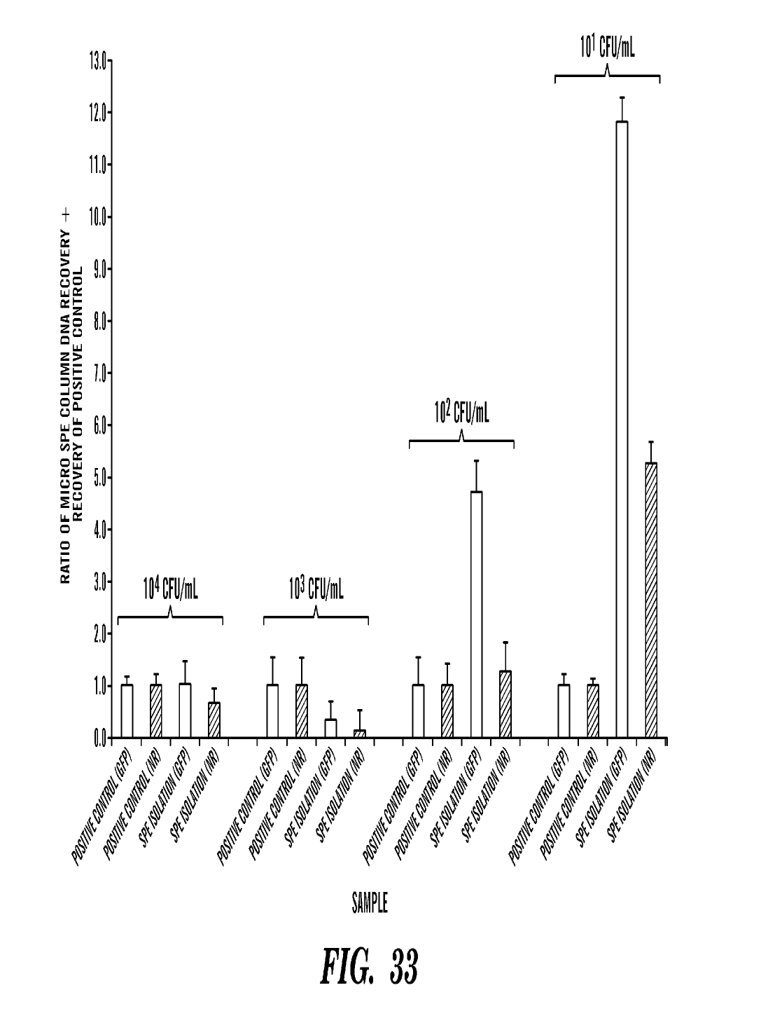

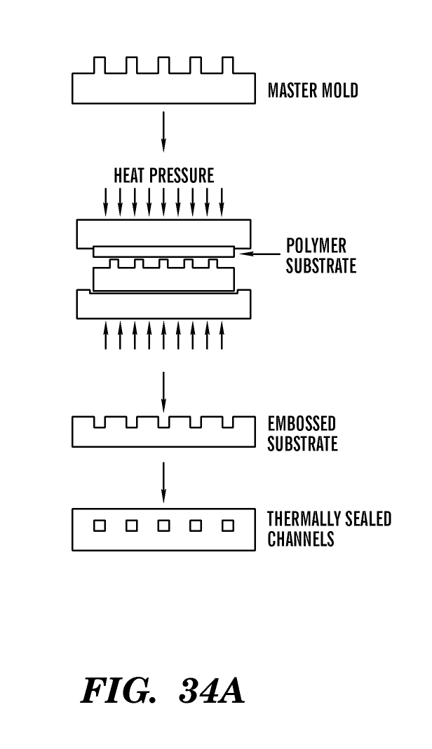

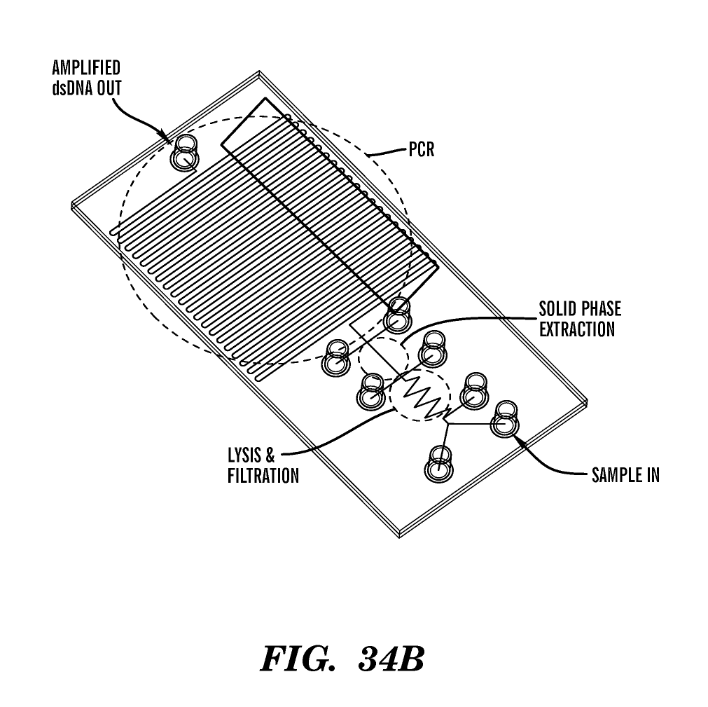

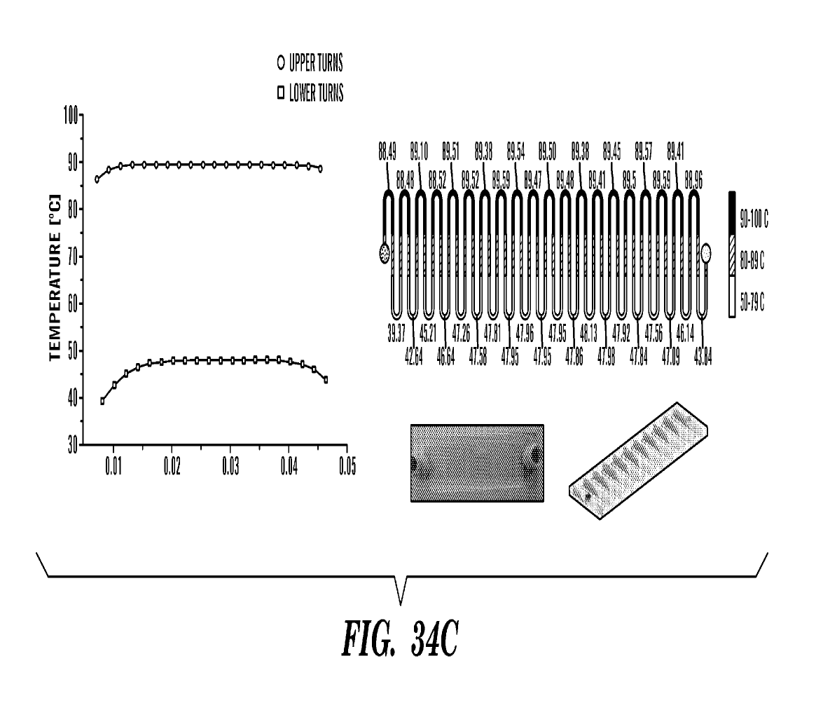

In order to study the porous polymer monoliths within the structure extensive imaging was done with both optical and scanning electron microscopes. As can be seen in FIG. 6, the optical microscope images show areas of light and dark within the channel, which are indicative of carbon nanotube clumping within the porous polymer structure. Further imaging was conducted using a Zeiss Scanning Electron Microscope (SEM). The images were taken by peeling off the cover layer and looking at the channel from above, with FIG. 7 shows an elevation view of a typical image of a porous polymer filled microfluidic channel. FIG. 8 shows an example of carbon nanotube clumping within the polymeric structure which was detected using a scanning electron microscope (SEM). The structures that resulted from the non-polar BUMA pre-polymer system demonstrated some characteristics ideal for the mechanical lysis application including instances where the nanotubes branch across the polymer pores, embodying the desired random configuration or random position of the carbon particles to result in a “barbed wire” effect, as can be seen in FIG. 9 and FIG. 27.

Fabrication Results: GMA Porous Polymer Monoliths.

The inventors fabricated polar GMA-based porous polymer monoliths and assessed their use for embedding carbon nanotubes. In order to fabricate the GMA monoliths, the inventors used optimal cross-linking time and energy levels ranging from 0.5 to 1.0 minutes per side and between 1000 and 1500 J/cm2 energy levels. The inventors discovered a cross-linking time of 0.7 minutes and energy of 1200 J/cm2 produced the best results. It was also observed that channel size seemed to impact the likelihood of success when fabricating the GMA-based monoliths, with smaller channels exhibiting flow characteristics indicative of excessive cross-linking. After cross-linking the GMA based porous polymer monoliths tended to be “blown out” of the channel during the washing step far more easily than those fabricated through the BUMA process. Between crosslinking difficulties and “blow-out” during washing then overall yield on GMA based monoliths was on the order of 25-30%, when compared to a near 75% yield on those fabricated with the BUMA process, which could be significantly improved by further process development and refinement.

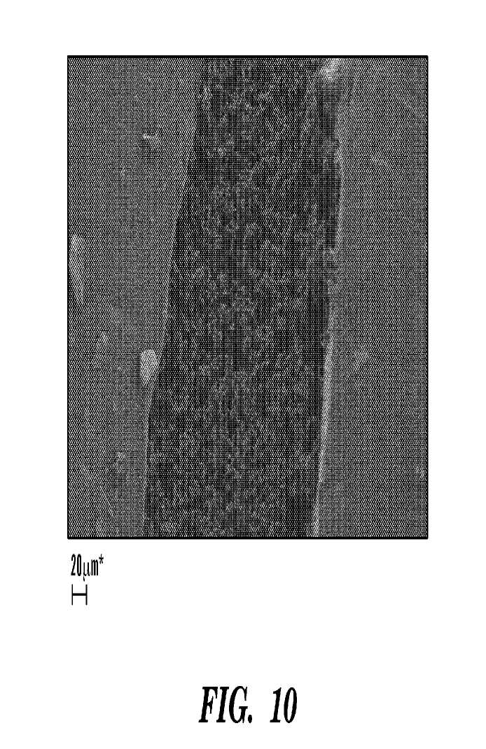

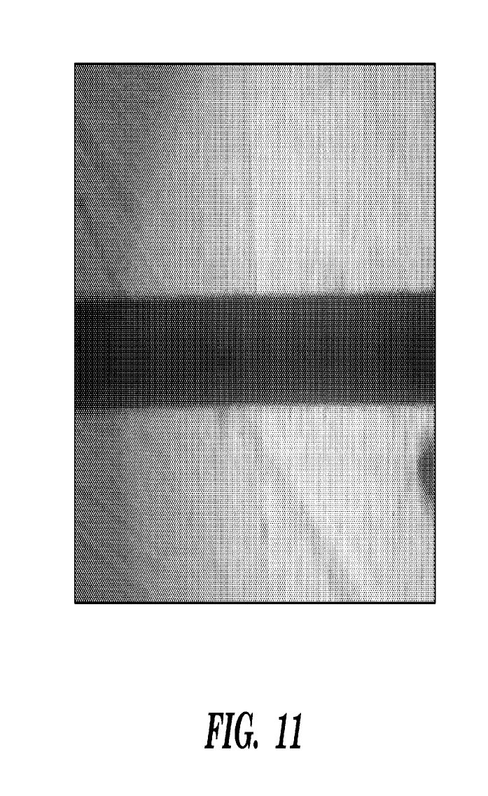

The inventors discovered polymer structures from the GMA system was not significantly different than those created by the BUMA system in terms of pore size or overall appearance, as shown in FIG. 11; however, it could be seen from the first optical microscope image shown in FIG. 10, that carbon nanotube dispersion appears superior in these monoliths as the overall color and appearance of the monolith is much more uniform and few dark patches indicative of large-scale clumping were visible.

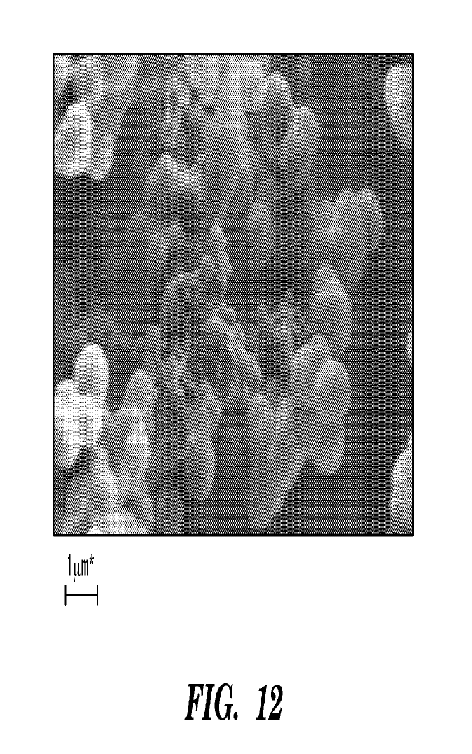

The inventors discovered it was difficult to locate the carbon nanotubes when examining the GMA based porous polymer monoliths, indicating that the carbon nanotubes were embedded within the polymer, as shown in FIG. 12 as an example of a suspected carbon nanotube with polymer wrapping.

Additional Porous Polymer Monolith Designs and Formulations.

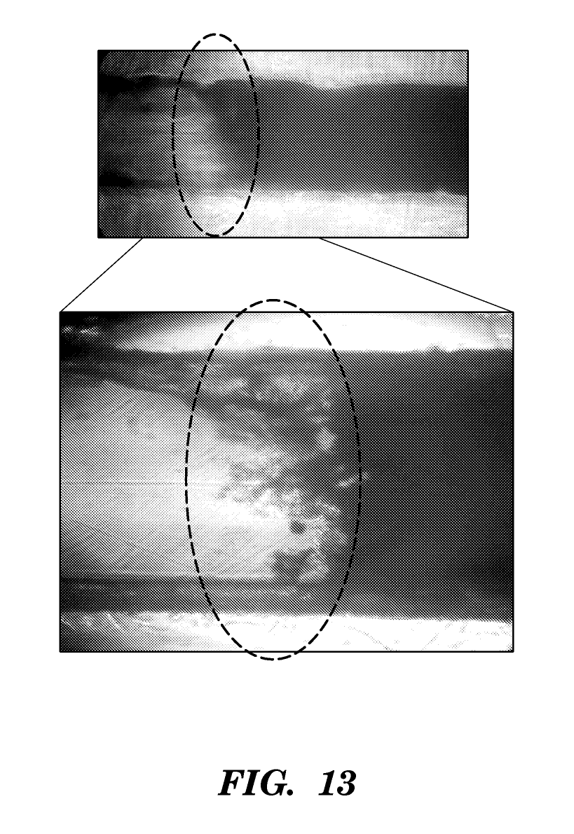

While the BUMA and GMA designs as previously described were used during device testing other designs and formulations were experimented with in parallel with testing to further materials and processes research. The inventors also discovered and tested an alternative design, based on the non-polar BUMA system, utilizing a “wall of nanotubes”. In this design, the inventors constructed a porous polymer monolith, (fabricated in accordance with the non-polar BUMA process and without embedded carbon nanotubes), which was fabricated within roughly one-half of the microfluidic channel and carbon nanotubes in cyclohexanol solvent were then flowed through the porous polymer monolith. The carbon nanotubes, tending to clump together in the non-polar cyclohexanol solvent, were captured on the front of the BUMA porous polymer monolith and created a wall of nanotubes through which a sample being tested. Images of the resulting structures are shown in FIG. 13, as imaged using an optical microscope.

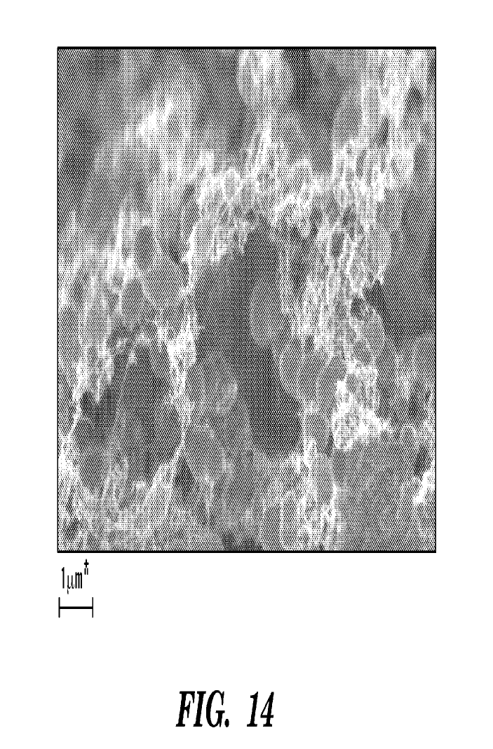

The inventors also discovered and tested another variant of the BUMA system, where methanol and ethanol was substituted for cyclohexanol and dodecanol as the constituent parts of the solvent system, (see formulation in Table 2). The inventors determined optimal cross-linking parameters of a cross-linking time of 0.7 minutes per side at an energy level of 1000 J. The inventors discovered these porous polymer monoliths tended to have combined properties between the BUMA fully non-polar and GMA polar systems. Using SEM images, the inventors discovered these monoliths comprising a combination of the non-polar monomer and polar solvents resulted in reasonable dispersion of the carbon nanotubes, although the carbon nanotubes residing primarily on the surface of the polymeric structure as shown in FIG. 14 and FIG. 29.

Table 2. pre-polymer solution used to formulate Hybrid polar/Non-polar polymer monolith

| | | Percentage of |
|---|---|---|
| | Alternate BUMA | Content by |
| | Formulation | Volume |
| Monomer | BUMA | 18% |
| Crosslinker | EDMA | 14% |
| Photoinitiator | DMPAP | 1% |
| Solvent 1 | .003M Ethanol w/CNTS | 27% |
| Solvent 2 | Methanol | 40% |

One can also use alternative polymers systems to create the porus polymer to embed the carbon particles according to the methods of the present invention, for example, such polymers but not limited to, listed in Table 4.

### Example 4

### Testing the Microfluidic Chips on Bacterial Lysis

The inventors formed microfluidic chips by hot-embossing a medical grade cyclic olefin polymer with a nickel-cobalt electroform master mold. A polymer monolith embedded with carbon nanotubes (CNTs) was formed by in situ UV polymerization and was used to perform on-chip mechanical lysis. Once lysed, we isolated the bacterial DNA using a silica bead/polymer composite solid-phase-extraction (SPE) column on-chip, as disclosed in U.S. Patent Application US2007/0015179, which is specifically incorporated herein in its entirety by reference and determined the presence of bacteria via fluorescence assays and real-time PCR.

TABLE 4
Alternative polymer monoliths that can be used in the methods of the present invention.

| Option | Microchannel | | | | |
|---|---|---|---|---|---|
| # | Substrate | Grafting Solution Used | Monomer | Solvents | Crosslinker |
| 1 | soda lime glass | 0.5% (v/v) 3-(trimethoxysilyl) | 1.42 g glycidyl | 3.6 g 50% ethanol/ | .96 g EDMA |
| | | propyl acrylate in toluene | methacrylate | 50% methanol (w/w) | |
| 2 | borofloat glass | 3-(trimethoxysilyl) propyl acrylate | .791 g BUMA | 2.52 g methanol, | .48 g EDMA |
| | | in acetone | | 1.08 g hexane | |
| 3 | silica capillaries | 3-(trimethoxysilyl) propyl acrylate | Butyl acrylate | (60% (wt) of pre- | EDMA (40% |
| | | in acetic acid | (59.4% of | polymer) Glacial | of monomer) |
| | | | monomer) | Acetic Acid and | |
| | | | | Methanol | |
| 4 | glass (non-specific) | 30% (v/v) 3-(trimethoxysilyl) | 24% (v/v) | dodecanol (60% v/v) | EDMA (15% |
| | | proxyl methacrylate in acetone | glycidyl | OR cyclohexanol | v/v) |
| | | | methacrylate | (54% v/v) with decyl | |
| | | | | alcohol (6% v/v) | |
| 5 | silica capillary tubing | 3-(trimethoxysilyl) propyl acrylate | Lauryl | 80% cyclohexanol/ | EDMA (35% |
| | | in acetic acid | methacrylate | 20% ethylene glycol | monomer |
| | | | (65% monomer | (comprises 60% total | molar ratio) |
| | | | molar ratio) | volume) | |
| 6 | COC, PMMA, PDMS | grafting was used for PDMS but it | Lauryl | methanol (30%) 2- | EDMA (16%) |
| | | doesn't talk about grafting with | methacrylate | propynol (30%) | |
| | | COC | (24%) | | |
| 7 | fused silica capillary | not discussed | 1.421 g Glycidyl | methanol (40-60%)/ | .96 g EDMA |
| | | | Methacrylate | ethanol (40-60%) | |
| 6 | fused silica capillary | not discussed | 1.301 g 2- | methanol (50%)/ | .96 g EDMA |
| | | | hydroxyethyl | ethanol (50%) | |
| | | | methacrylate | | |
| 9 | fused silica capillary | not discussed | 1.422 g BUMA | methanol (50%)/ | .96 g EDMA |
| | | | | ethanol (50%) | |

| Option | | | | |
|---|---|---|---|---|
| # | Initiator | Other | Processing | Reference |
| 1 | .024 g | 2-acrylamido-2-methyl-1- | 30 min grafting reaction (no UV). | Ramsey, Collins, Anal. Chem, |
| | azobisisobutyronitrile | propane sulfonic acid (EOF | 45 min UV polymerization for | 2005, 77, 6664-6670 |
| | | promoter) | monolith | |
| 2 | 12 mg | none | 24 hour graft (in the dark). 3 hr | Yu, Davey, Svec, Frechet, |
| | azobisisobutyronitrile | | UV polymerization for monolith | Anal Chem, 2001, 73, 5088- |
| | | | | 5096 |
| 3 | Benzoin methyl ether | 2-acrylamido-2-methyl-1- | 60 min grafting reaction. 10-30 | Koerner, Turck, et al. Anal |
| | (.6% (wt) of | propane sulfonic acid (EOF | min UV polymerization | Chem, 2004, 75, 6456-6460 |
| | monomer) | promoter, 0.5% (wt) of | | |
| | | monomer) | | |
| 4 | DMPAP (1% w/v) | none | Overnight grafting reaction. 6 | Mao, Luo et al. Anal Chem, |
| | | | min UV polmerization | 2004, 76, 6941-6947 |
| 5 | 1% (wt of monomers) | none | not specified | Le Gac, Carlier et al. J. |
| | azobisisobutyronitrile | | | Chromo B, 808, 2004, 3-14 |
| 6 | Benzoin methyl ether | none | none or grafting with COC. 8 W | Bedair, Oelschuk, Anal Chem, |
| | (.4%) | | @365 nm for 15 min for UV | 2006, 78, 1130-1138 |
| | | | polymerization | |
| 7 | AIBN (1% by weight | none | UV initiation @ 365 nm for 16 h, | Yu, Xu, Svec, Frechet, Jol |
| | of monomers) | | room temp | Polymer Sc, 2002, 40, 755- |
| | | | | 767 |
| 6 | AIBN (1% by weight | none | UV initiation @ 365 nm for 16 h, | Yu, Xu, Svec, Frechet, Jol |
| | of monomers) | | room temp | Polymer Sc, 2002, 40, 755- |
| | | | | 767 |
| 9 | AIBN (1% by weight | none | UV initiation @ 365 nm for 16 h, | Yu, Xu, Svec, Frechet, Jol |
| | of monomers) | | room temp | Polymer Sc, 2002, 40, 755- |
| | | | | 767 |

In order to conduct testing of the fabricated devices a test organism needed to be chosen since use of C. Difficile, a BSL 2 bacteria, was deemed unnecessarily dangerous for use in the first round of testing. A thorough review of candidate substitute organisms was completed to find the closest biological and geometric match possible in a BSL 1 organism. C. Difficile possesses several key characteristics that would need to be considered in selecting any test organism in order to be confident that device performance demonstrated with the test organism would translate over when testing with C. Difficile began. First off, C. Difficule is a gram-positive bacterium, which means that it possesses an additional outer membrane and a thicker layer of peptidoglycan, which is made of a protein-sugar complex that is believed to lead to a stronger overall cell wall than in gram-negative bacteria. C. Difficile is a bacillus shaped (cylindrical), bacteria approximately ¾ of a micron in diameter and ˜5 microns long. The test organism should closely resemble C. Difficile in shape and size. Other secondary factors considered included growth conditions, (whether it would be straight forward to grow), whether the test organism is aerobic or anaerobic, whether it was endospore forming, and whether its replication processes resulted in the formation of chains.

As a result of the study two bacteria were chosen to be used during testing. Wild Type E. Coli, (a gram-negative bacteria donated by Hemali Patel of Boston University), to begin testing and determine initial device effectiveness, (which in theory should be better with the easier-to-lyse gram negative organism). A special strain of non-chain forming Bacillus Subtilis, (Strain 168, donated by Shigeki Moriya, Institute for the Biotechnology of Infectious Diseases in Sydney, Austrailia), was chosen to simulate C. Difficile. Table 3 shows a summary the organisms considered and comparisons made.

Bacterial Cell Culture In order to utilize the bacteria in testing of the microfluidic devices a liquid culture of the bacteria was prepared in a growth media. The bacteria to be cultured were first cultured on an agar plate by using an applicator to streak the plate from another culture. Once colonies begin to form a single colony of the bacteria is used to begin the liquid culture In the case of Wild-Type E. Coli, a single colony is introduced to 3-5 ml of Luria-Bertani, (LB), bacterial growth media. In the case of Strain 168 B. Subtilis a media comprised on DIFCO Antibiotic Medium 3, including an additional 20 mg of each Adenine and Guanosine in 1 mililiter of NaOH were used based upon the recommendation of the donator to ensure that the bacteria exhibited the non-chain forming replication it was modified for. After placing the bacteria in the desired media it is then typically incubated overnight, (14-16 hours), at 37° C., although it is noteworthy to mention that in these experiments the incubation was carried on for longer periods, (usually 24 hrs⁺) to ensure that the bacteria reach stationary phase in which they are no longer replicating. Bacterial cell counts of ˜105-109 cells per milliliter were cultured in this fashion. An approximate cell count was obtained by utilizing the colony forming units method, (CFU), where the culture is serial diluted and then 10 ul of each serial dilution, (typically from 104-109 cells), were plated in triplicate on an agar plate. The number of colonies resulting from each 10 ul droplet served as an indication as to the average cell concentration within the culture.

TABLE 3
Test Organisms Considered to simulate C. Difficile⁴⁷.
Comparison of Potential Test Organisms

| | C. Difficile | E. Coli | B. Cereus | Lactobacillus | B. Subtilis | B. Subtilis (Strain 168) |
|---|---|---|---|---|---|---|
| Type | gram positive | gram negative | gram positive | gram positive | gram positive | gram positive |
| Diameter (um, typ) | 0.75 | 0.1-0.5 | 1.0-1.2 | 0.5-2.0 | 0.5-1.0 | 0.5-1.0 |
| Length (um, typ) | 1.0-5.0 | 1.0-2.0 | 3.0-5.0 | 1.0-9.0 | 1.0-3.0 | 1.0-3.0 |
| Aerobic? | N | N | Y | N | Y | Y |
| Spore Forming? | Y | N | Y | N | Y | Y |
| Chain Forming? | N | N | N | Y | Y | N |

The inventors investigated both, gram-negative and gram-positive bacteria, for on-chip lysis and found positive results in comparison to current bench techniques. The inventors cultured Escherichia coli, a gram-negative bacteria, and a non-chain forming Bacillus subtilis, a gram-positive bacteria. The bacteria was resuspended in a chaotropic buffer with detergent. This was driven through the CNT channels using pressure. While suspending the bacteria in the chaotropic buffer and detergent aided lysis chemically, the channel aided lysis with mechanical force. Once driven through the CNT monolith, the sample was driven through a SPE column where the DNA was isolated. By coupling bacterial lysis on-chip to DNA isolation on-chip, our device has the potential to facilitate fast diagnosis and treatment of bacterial infections.

BUMA Porous Polymer Monolith Microfluidic Device for Testing E. Coli Bacteria.

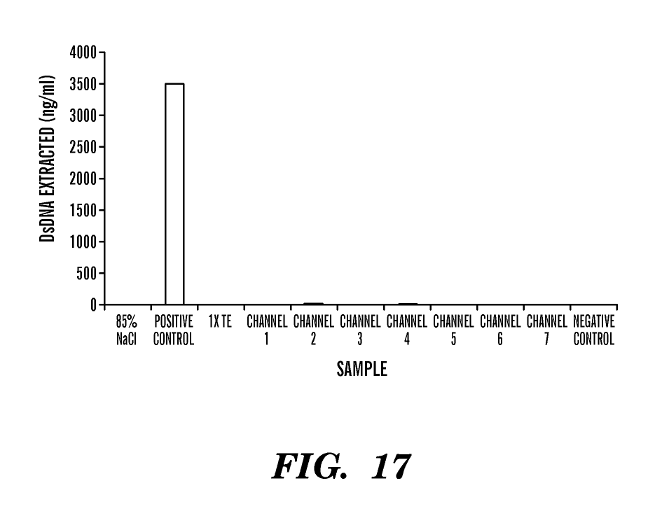

The inventors assessed the BUMA porous polymer monoliths to lyse the test bacteria E. Coli. E. Coli was suspended in 0.85% sodium chloride and water. The inventors discovered minimal lysis with only two of seven channels tested indicating lysis, as shown in FIG. 17. Additional controls were run for the media and 1×TE to double check that they were not contributing to fluorescence. The two channels that lysis occurred, the inventors discovered the extracted concentrations of DNA on the order of tens of nanograms of DNA per milliliter, two orders of magnitude less than those extracted using the Qiagen protocol. The quantity of dsDNA extracted was evaluated using two separate standard curves, (high range and low range), in order to provide maximum accuracy.

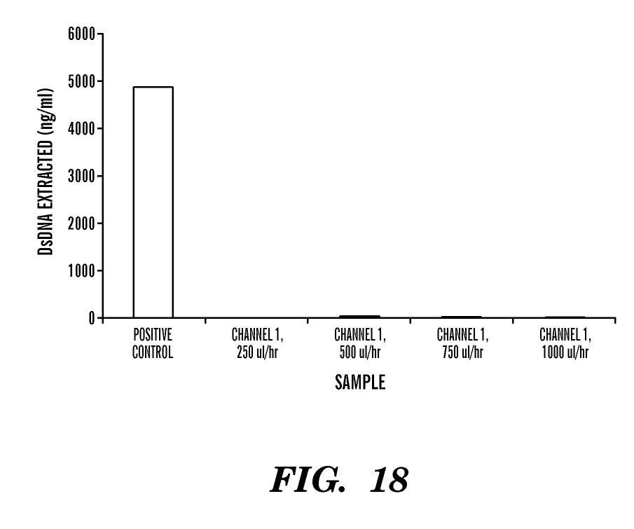

In additional experiments, the inventors discovered optimal flow velocity and increased supply pressure at the inlet to the channel for increased effectiveness of lysis by the device. The inventors assessed three new channels at all four different flow rates, with one of the three channels successful at being used at all four flow rates. The inventors discovered that increased flow rate did not increase the devices ability to lyse cells and resulted in decreased repetitive use of the device. Thus, the inventors discovered the optimal flow rate was a low flow rate, which was utilized throughout the rest of the testing conducted, as shown in FIG. 18 demonstrating channel results tested at all four flow rates.

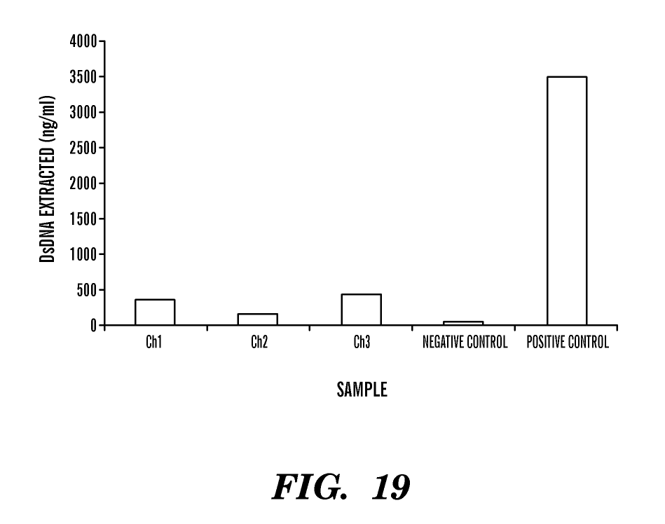

The inventors also tested the BUMA device to lyse cells when the cells were suspended in Qiagen Buffer B1 prior to lysis using the BUMA device. The inventors discovered that bacterial cells suspended in a detergent based buffer prior to lysis using the device or the present invention resulted in increased levels of lysis as compared to without the addition of the buffer including detergents. The inventors discovered that cell lysis and yield was improved by two orders of magnitude as compared to use of the same device design with cells run through it suspended in 0.85% sodium chloride. The inventors also discovered that at least twice and as much as four times as high a concentration of dsDNA was obtained as compared with the device used when the sample is the negative control as shown in FIG. 19. The inventors also performed tests on the device using both 0.85% sodium chloride and buffer B1 suspended cells multiple times (at least twice) to ensure repeatability. All BUMA devices tested were used only once and disposed of after use.

GMA Porous Polymer Monolith Microfluidic Device for Testing E. Coli Bacteria.

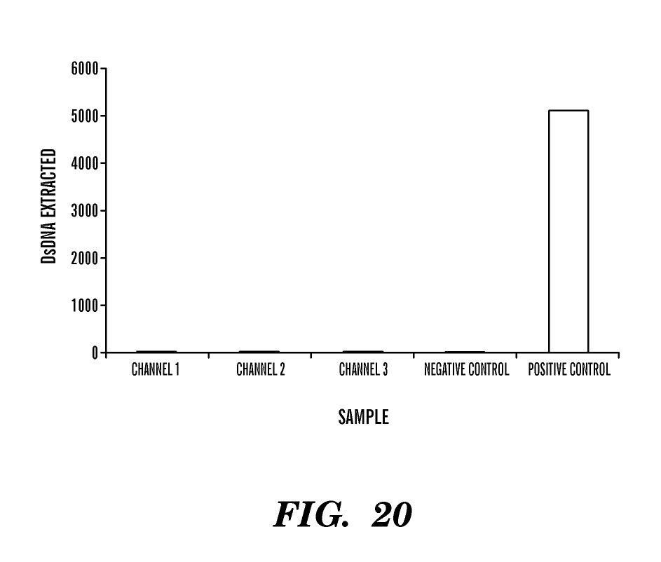

After completing testing of the BUMA based with E. Coli the inventors assessed the GMA based devices to determine their performance. The inventors conducted experiments with suspensions of cells in both 0.85% sodium chloride and buffer B1. The GMA based devices were subsequently tested for lysis of B. Subtilis for comparative purposes, where cells were suspended in either 0.85% NaCl or buffer B1 prior to lysis using the GMA device. Lysis of cell suspensions in 0.85% NaCl using the GMA devices resulted in a yield of between 16-20 ng/ml of dsDNA, as shown in FIG. 20.

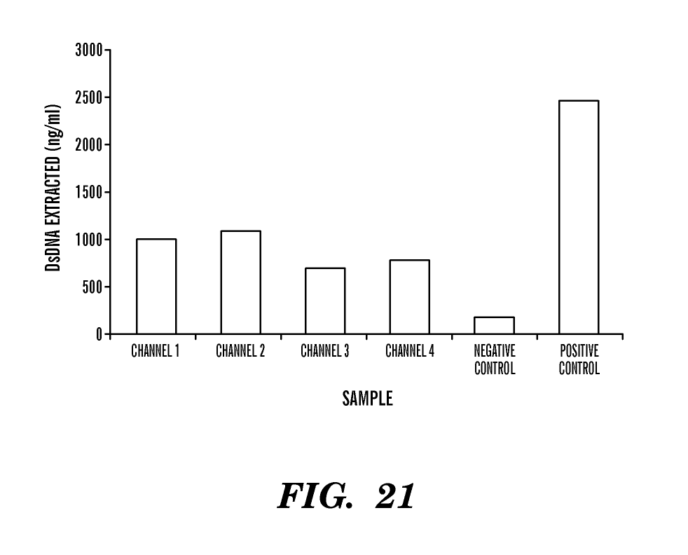

The inventors discovered that, as seen with the BUMA device, cell lysis was significantly increased using the GMA device of cells suspended in buffer B1 as compared to 0.85% NaCl. The inventors also discovered that the DNA yield from cells lysed using the GMA based devices was approximately twice the yield from cells lysed using the BUMA based devices when the cells are prior suspended in buffer B1, with dsDNA concentrations extracted ranging from ˜750 ng/ml to just over 1000 ng/ml using the GMA device as compared to ˜250 ng/ml to just over 500 ng/ml using the BUMA device. As also seem with the BUMA device, the inventors discovered the GMA device exhibited significantly more lysis than the negative control, extracting at a minimum three times higher concentrations and as much as nearly five times as high a concentration as the negative control as shown in FIGS. 20 and 21.

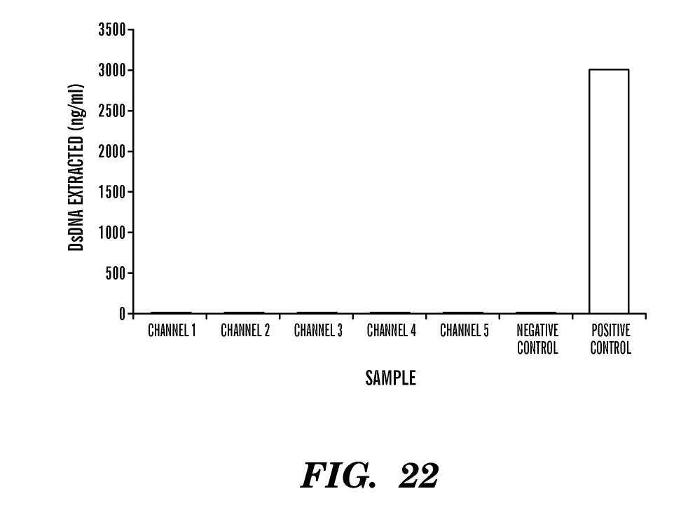

The inventors discovered that the GMA device was less effective at lysing B. Subtilis suspended in 0.85% NaCl, with extracted dsDNA concentrations ranging betweem ˜3-8 ng/ml and the negative control indicated that the device did not appreciably improve lysis efficiency, (see FIG. 22).

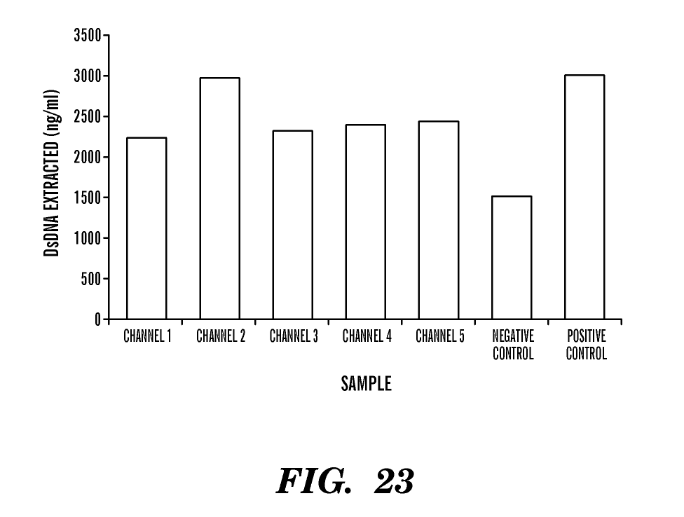

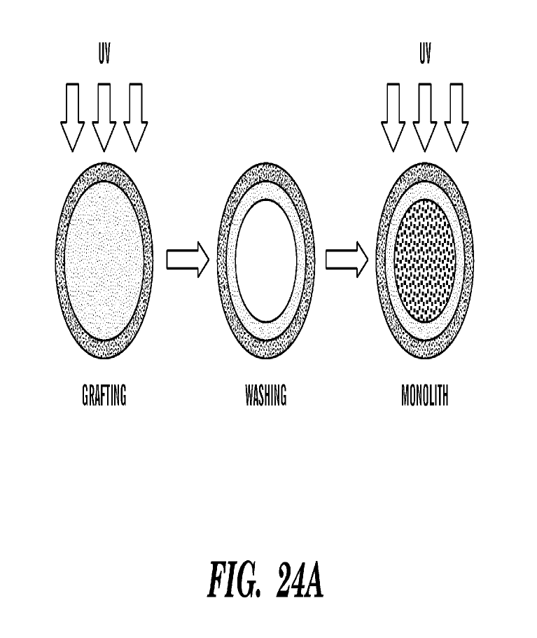

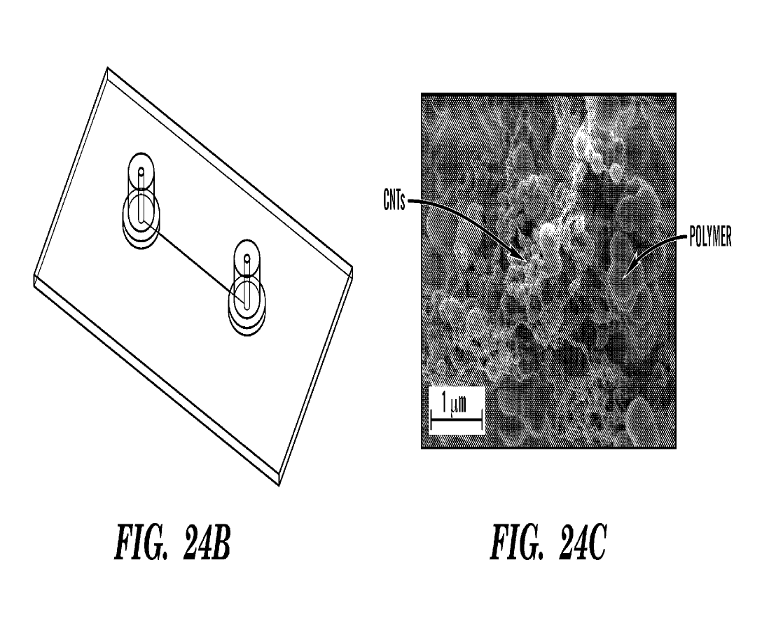

The inventors also discovered that the GMA device was more effective in lysing B. Subtilis suspended in buffer B1 than they were at lysing E. Coli suspended in buffer B1, with successful extraction of a yield of DNA at a similar high a concentration of dsDNA that was extracted by the positive Qiagen protocol control, as shown in FIG. 23. The inventors also discovered that GMA devices, if necessary, can be used multiple times and washed with methanol and water between each use.

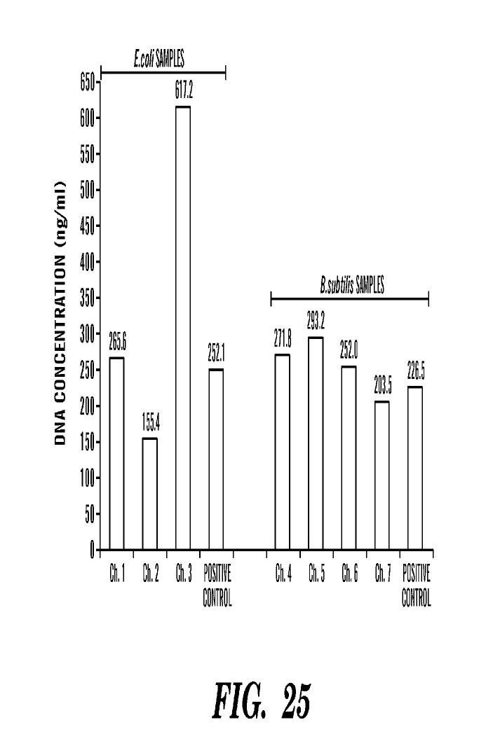

Both, gram-negative and gram-positive bacteria were mechanically lysed on a microfluidic chip using a polymer monolith embedded with CNTs (FIG. 25). After establishing mechanical lysis, we moved forward with isolating the bacterial dsDNA on-chip for downstream applications.

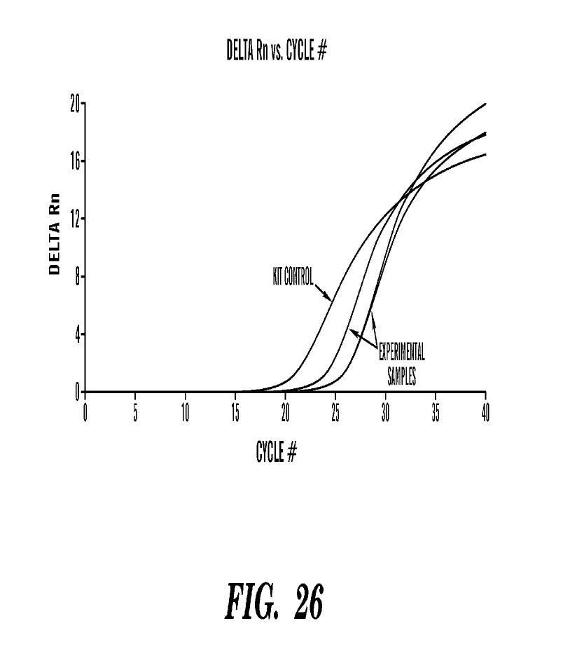

The bacteria were then suspended in a chaotropic buffer, detergent, and Proteinase K and were lysed on-chip followed by isolating the bacterial dsDNA from the cell-lysate by using a SPE column. After washing the SPE column the isolated DNA was eluted in water. This DNA was amplified using real-time PCR and compared to the kit standard (FIG. 26).

The inventors have discovered a comparable system of lysis to the current standard bench-top kit for bacterial lysis. The inventors have discovered a method for bacterial lysis using a lysis column can be fabricated serially with a solid-phase extraction column to streamline lysis and DNA isolation sample preparation techniques on a single-platform.

With this system, the inventors have established the proof of concept of on-chip bacterial lysis for non-infectious bacteria and can move forward with investigating C. difficile on-chip lysis. Future work includes working with clinical stool samples to determine specific infection with C. difficile.

### REFERENCES

The references cited herein and throughout the application are incorporated herein by reference.
1. Frost and Sullivan Company. 2005 “US Genetic Diagnostics Market Report (F463-52)”. Frost and Sullivan Market Reports, Retrieved May 2006. p 1-2. (www.frost.com)
2. Pothoulakis, M. D. 2001. “Clostridium Difficile Infection.” Participate, Retrieved February 2006, pp. 1-3. (http://www.aboutibs.org/Publications/CDifficile.html)
3. Wheeldon, Laura. 2005. “Clostridium Difficile: Return of the Old Enemy.” Microbiologist pp. 33-35.
4. Pothoulakis, M. D. 2001. “Clostridium Difficile Infection.” Participate, Retrieved February 2006, pp. 1-3. (http://www.aboutibs.org/Publications/CDifficile.html)
5. Wheeldon, Laura. 2005. “Clostridium Difficile: Return of the Old Enemy.” Microbiologist pp. 33-35.
6. Pothoulakis, M. D. 2001. “Clostridium Difficile Infection.” Participate, Retrieved February 2006, pp. 1-3. (http://www.aboutibs.org/Publications/CDifficile.html)
7. Wang, Jing., Mauk, Michael G., Chen, Zongyuan., Bau, Haim H. 2005. “A disposable microfluidic device for cell lysis and DNA isolation.” 9th International Conference on Miniturized Systems for Chemistry and Life Sciences. pp. 289-291
8. El-Ali, J., Murphy Jr., K. P., Nielsen, U. B., Jensen, K. F. 2005. “Microfluidic device with integrated antibody arrays for cell signalling analysis.” 9th International Conference on Miniturized Systems for Chemistry and Life Sciences. pp. 388-390.
9. Sethu, Palaniappan., Anahtar, Melis., Moldawer, Lyle., Tompkins, Ronald., Toner, Mehmet. 2004. “Continuous flow microfluidic device for rapid erythrocyte lysis.” Anal. Chem. 76:6247-6253.
10. Heo, Jinseok., Thomas, K. Joseph., Seong, Gi Hun., Crooks, Richard M. 2003. “A microfluidic bioreactor based on hydrogel-entrapped E. coli: Cell viability, lysis, and intracellular enzyme reactions.” Anal. Chem. 75:22-26.
11. Schilling, Eric A., Kamholz, Andrew Evan., Yager, Paul. 2002. “Cell lysis and protein extraction in a microfluidic device with detection by a fluorogenic enzyme assay.” Anal. Chem. 74:1798-1804.
12. Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
13. Di Carlo, Dino., Jeong, Ki-Hum., Lee, Luke. P. 2003. “Reagentless mechanical cell lysis by nanoscale barbs in microchannels for sample preparation.” Lab on a Chip. 3:287-291.
14. Taylor, Michael T., Belgrader, Phillip., Furman, Burford., Pourahmadi, Farzad., Kovacs, Gregory., Northrop, M. Allen. 2001. “Lysing bacterial spores by sonication through a flexible interface in a microfluidic system.” Anal. Chem. 73:492-496.
15. Devitt, Amy J., Aflatooni, Nima., Vinas, Mary., Loh, Nin., Pourahmadi, Farzad., Yuan, Robert., Northrop, M. Allen. “Differential extraction of male and female DNA in an automated microfluidic device.” 9th International Conference on Miniturized Systems for Chemistry and Life Sciences. pp. 7-9.
16. Liu, Robin Hui., Yang, Jianing., Lenigk, Ralf., Bonanno, Justin., Grodzinski. 2004. “Self-contained, fully integrated biochip for sample preparation, polymerase chain reaction amplification and DNA microarray detection.” Anal. Chem. 76: 1824-1831.
17. Wang, Tza-Huei., Chen, Yih-Far., Masset, Sylvain., Ho, Chih-Ming., Tai, Yu-Chong. 2000. “Molecular beacon based micro biological detection system.” International conference on mathematics and engineering techniques in medicine and biological sciences.
18. Hellmich, W., Leffhalm, K., Sischka, A., Duong, T., Jensen, N., Niehaus, K., Tonsing, K., Ros, A., Anselmetti, D. 2005. “Towards single cell fingerprinting in microfluidic device format: single cell manipulation, protein separation and detection.” 9th International Conference on Miniturized Systems for Chemistry and Life Sciences. pp. 406-408.
19. Poulsen, Claus R., Ramsey, J. Michael. 2005. “Continuous single cell lysis with integrated separation of cell content.” 9th International Conference on Miniturized Systems for Chemistry and Life Sciences. pp. 880-882.
20. Lu, Hang., Schmidt, Martin A., Jensen., Klays F. 2005. “A microfluidic electroporation device for cell lysis.” Lab on a Chip 5:23-29.
21. McClain, Maxine A., Culbertson, Christopher C., Jacobson, Stephen C., Allbritton, Nancy L., Sims, Christopher E., Ramsey, J. Michael. 2003. “Microfluidic devices for the high-throughput chemical analysis of cells.” Anal. Chem. 75:5646-5655.
22. Lagally, E. T., Lee, S. H., Soh, H. T. 2005. “Integrated microsystem for electrokenetic cell concentration and genetic detection.” 9th International Conference on Miniturized Systems for Chemistry and Life Sciences. pp. 1407-1409.
23. Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
24. Wang, Jing., Mauk, Michael G., Chen, Zongyuan., Bau, Haim H. 2005. “A disposable microfluidic device for cell lysis and DNA isolation.” 9th International Conference on Miniturized Systems for Chemistry and Life Sciences. pp. 289-291.
25. Devitt, Amy J., Aflatooni, Nima., Vinas, Mary., Loh, Nin., Pourahmadi, Farzad., Yuan, Robert., Northrop, M. Allen. “Differential extraction of male and female DNA in an automated microfluidic device.” 9th International Conference on Miniturized Systems for Chemistry and Life Sciences. pp. 7-9. Dadic, D., Doffing, F., Herrmann, M., Munchow, G., Drese, K. S. 2005. “On-chip blood sample preparation for subsequent PCR.” 9th International Conference on Miniturized Systems for Chemistry and Life Sciences. pp. 811-813.
27. Hashioka, Shingi., Ogawa, Ryo., Oki, Akio., Miyahara, Yuji., Horiike, Yasuhiro. 2005. “Simple and quick detection of target DNA by hybridization in nano gap channel array.” 9th International Conference on Miniturized Systems for Chemistry and Life Sciences. pp. 730-732.
28. Fouque, B., Brachet, A. G., Marcel, F., Dupont, R., Delapierre, G., Fischetti, A., Falava, J., Chatelain, F. 2005. “A lab-on-chip for rapid DNA-based identification of streptococcus pneumoniae.” 9th International Conference on Miniturized Systems for Chemistry and Life Sciences. pp. 292-294.
29. Ferrance, Jerome P., Bienvenue, Joan., Legendre, Lindsay., Easley, Chris., Karlinsey, James., Roper,
Michael G., Landers, James P. 2005. “A fully integrated microdevice for clinical analysis.” 9th International Conference on Miniturized Systems for Chemistry and Life Sciences. pp. 292-294.
30. Wang, Jing., Mauk, Michael G., Chen, Zongyuan., Bau, Haim H.2005. “A disposable microfluidic device for cell lysis and DNA isolation.” 9th International Conference on Miniturized Systems for Chemistry and Life Sciences. pp. 289-291.
31. Apte, P. et al., 2005. “Micro-miniaturized electophoresis DNA separator using MEMS,” Proceedings of SPIE, 5718:276-282.
32. Hsiung, S. K. et al 2005., “A microfabricated capillary electrophoresis chip multiple buried optical fibers and microfocusing lens for multiwavelength detection,” Electrophoresis 5:1122-1129.
33. Di Carlo, Dino., Jeong, Ki-Hum., Lee, Luke. P. 2003. “Reagentless mechanical cell lysis by nanoscale barbs in microchannels for sample preparation.” Lab on a Chip. 3:287-291.
34. http://www.seldontechnologies.com
35. Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
36. Yu, Min-Feng et. al. 2000. “Strength and Breaking Mechanism of Multiwalled Carbon Nanotubes Under Tensile Load.”, Science 287:637-640
37. Collins, Philip G., Avouris, Phaedon. 2000. “Nanotubes for Electronics.” Scientific American. December 2000, p. 68
38. http://www.ewels.info
39. Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
40. Yu, Cong., Xu, Mingcheng., Svec, Frantisek., Frechet, M. J., 2002. “Preparation of monolithic polymers with controlled porous properties for microfluidic chip applications using photointiated free-radical polymerization.” Journal of Polymer Science: Part A: Polymer Chemistry 40:755-769.
41. Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
42. Yu, Cong., Xu, Mingcheng., Svec, Frantisek., Frechet, M. J., 2002. “Preparation of monolithic polymers with controlled porous properties for microfluidic chip applications using photointiated free-radical polymerization.” Journal of Polymer Science: Part A: Polymer Chemistry 40:755-769.
43. Schäffer, M. S. P., Fan, X., Windle, H.1997. “Dispersion and Packing of Carbon Nanotubes.” Carbon 36: 1603-1612.
44. Lu, K. L., Lago, R. M., Chen, Y. K., Green, M. L. H., Harris, P. J. F., Tsang., S. C. 1996. “Mechanical damage of carbon nanotubes by ultrasound.” Carbon 36:814
45. Bhattacharyya, Arpita., Klapperich, Catherine. 2005. “Polymeric microfluidic device for on-chip cell
lysis and extraction of nucleic acids from biological samples.” 9th International Conference on Miniturized Systems for Chemistry and Life Sciences. pp. 1167-1169.
46. Ibid.
47. Laskin, Allan I and Lechevalier, Hubert A. 1973. CRC Handbook of Microbiology (Volume I). CRC Press.
Apte, P. et al., 2005. “Micro-miniaturized electophoresis DNA separator using MEMS,” Proceedings of SPIE, 5718:276-282.
Bhattacharyya, Arpita., Klapperich, Catherine. 2005. “Polymeric microfluidic device for on-chip cell lysis and extraction of nucleic acids from biological samples.” 9th International Conference on Miniturized Systems for Chemistry and Life Sciences. pp. 1167-1169.
Collins, Philip G., Avouris, Phaedon. 2000. “Nanotubes for Electronics.” Scientific American. December 2000, p. 68
Devitt, Amy J., Aflatooni, Nima., Vinas, Mary., Loh, Nin., Pourahmadi, Farzad., Yuan, Robert., Northrop, M. Allen. “Differential extraction of male and female DNA in an automated microfluidic device.” 9th International Conference on Miniturized Systems for Chemistry and Life Sciences. pp. 7-9.
Di Carlo, Dino., Jeong, Ki-Hum., Lee, Luke. P. 2003. “Reagentless mechanical cell lysis by nanoscale barbs in microchannels for sample preparation.” Lab on a Chip. 3:287-291.
Dadic, D., Doffing, F., Herrmann, M., Munchow, G., Drese, K. S. 2005. “On-chip blood sample preparation for subsequent PCR.” 9th International Conference on Miniturized Systems for Chemistry and Life Sciences. pp. 811-813.
El-Ali, J., Murphy Jr., K. P., Nielsen, U. B., Jensen, K. F. 2005. “Microfluidic device with integrated antibody arrays for cell signalling analysis.” 9th International Conference on Miniturized Systems for Chemistry and Life Sciences. pp. 388-390.
Ferrance, Jerome P., Bienvenue, Joan., Legendre, Lindsay., Easley, Chris., Karlinsey, James., Roper, Michael G., Landers, James P. 2005. “A fully integrated microdevice for clinical analysis.” 9th International Conference on Miniturized Systems for Chemistry and Life Sciences. pp. 292-294.
Fouque, B., Brachet, A. G., Marcel, F., Dupont, R., Delapierre, G., Fischetti, A., Falava, J., Chatelain, F. 2005. “A lab-on-chip for rapid DNA-based identification of streptococcus pneumoniae.” 9th International Conference on Miniturized Systems for Chemistry and Life Sciences. pp. 292-294. 98
Frost and Sullivan Company. 2005 “US Genetic Diagnostics Market Report (F463-52)”. Frost and Sullivan Market Reports, Retrieved May 2006. p 1-2. (www.frost.com)
Hashioka, Shingi., Ogawa, Ryo., Oki, Akio., Miyahara, Yuji., Horiike, Yasuhiro. 2005. “Simple and quick detection of target DNA by hybridization in nano gap channel array.” 9th International Conference on Miniturized Systems for Chemistry and Life Sciences. pp. 730-732.
Hellmich, W., Leffhalm, K., Sischka, A., Duong, T., Jensen, N., Niehaus, K., Tonsing, K., Ros, A., Anselmetti, D. 2005. “Towards single cell fingerprinting in microfluidic device format: single cell manipulation, protein separation and detection.” 9th International Conference on Miniturized Systems for Chemistry and Life Sciences. pp. 406-408.
Heo, Jinseok., Thomas, K. Joseph., Seong, Gi Hun., Crooks, Richard M. 2003. “A microfluidic bioreactor based on hydrogel-entrapped E. coli: Cell viability, lysis, and intracellular enzyme reactions.” Anal. Chem. 75:22-26.
Hsiung, S. K. et al 2005., “A microfabricated capillary electrophoresis chip multiple buried optical fibers and microfocusing lens for multiwavelength detection,” Electrophoresis 5:1122-1129
Lagally, E. T., Lee, S. H., Soh, H. T. 2005. “Integrated microsystem for electrokenetic cell concentration and genetic detection.” 9th International Conference on Miniturized Systems for Chemistry and Life Sciences. pp. 1407-1409.
Laskin, Allan I and Lechevalier, Hubert A. 1973. CRC Handbook of Microbiology
(Volume I). CRC Press.
Liu, Robin Hui., Yang, Jianing., Lenigk, Ralf., Bonanno, Justin., Grodzinski. 2004. “Self-contained, fully integrated biochip for sample preparation, polymerase chain reaction amplification and DNA microarray detection.” Anal. Chem. 76: 1824-1831.
Lu, Hang., Schmidt, Martin A., Jensen., Klays F. 2005. “A microfluidic electroporation device for cell lysis.” Lab on a Chip 5:23-29.
Lu, K. L., Lago, R. M., Chen, Y. K., Green, M. L. H., Harris, P. J. F., Tsang., S. C. 1996. “Mechanical damage of carbon nanotubes by ultrasound.” Carbon 36:814 99
McClain, Maxine A., Culbertson, Christopher C., Jacobson, Stephen C., Allbritton, Nancy L., Sims, Christopher E., Ramsey, J. Michael. 2003. “Microfluidic devices for the high-throughput chemical analysis of cells.” Anal. Chem. 75:5646-5655.
Pothoulakis, M. D. 2001. “Clostridium Difficile Infection.” Participate, Retrieved February 2006, pp. 1-3. (http://www.aboutibs.org/Publications/CDifficile.html)
Poulsen, Claus R., Ramsey, J. Michael. 2005. “Continuous single cell lysis with integrated separation of cell content.” 9th International Conference on Miniturized Systems for Chemistry and Life Sciences. pp. 880-882.
Schaffer, M. S. P., Fan, X., Windle, H.1997. “Dispersion and Packing of Carbon Nanotubes.” Carbon 36: 1603-1612.
Schilling, Eric A., Kamholz, Andrew Evan., Yager, Paul. 2002. “Cell lysis and protein extraction in a microfluidic device with detection by a fluorogenic enzyme assay.” Anal. Chem. 74:1798-1804.
Sethu, Palaniappan., Anahtar, Melis., Moldawer, Lyle., Tompkins, Ronald., Toner, Mehmet. 2004. “Continuous flow microfluidic device for rapid erythrocyte lysis.” Anal. Chem. 76:6247-6253.
Taylor, Michael T., Belgrader, Phillip., Furman, Burford., Pourahmadi, Farzad., Kovacs, Gregory., Northrop, M. Allen. 2001. “Lysing bacterial spores by sonication through a flexible interface in a microfluidic system.” Anal. Chem. 73:492-496.
Wang, Jing., Mauk, Michael G., Chen, Zongyuan., Bau, Haim H. 2005. “A disposable microfluidic device for cell lysis and DNA isolation.” 9th International Conference on Miniturized Systems for Chemistry and Life Sciences. pp. 289-291
Wang, Tza-Huei., Chen, Yih-Far., Masset, Sylvain., Ho, Chih-Ming., Tai, Yu-Chong. 2000. “Molecular beacon based micro biological detection system.” International conference on mathematics and engineering techniques in medicine and biological sciences.
Wheeldon, Laura. 2005. “Clostridium Difficile: Return of the Old Enemy.” Microbiologist pp. 33-35.
Yu, Cong., Xu, Mingcheng., Svec, Frantisek., Frechet, M. J., 2002. “Preparation of monolithic polymers with controlled porous properties for microfluidic chip applications using photointiated free-radical polymerization.” Journal of Polymer Science: Part A: Polymer Chemistry 40:755-769.
Yu, Min-Feng et. al. 2000. “Strength and Breaking Mechanism of Multiwalled Carbon Nanotubes Under Tensile Load.”, Science 287:637-640 101

1. A microfluidic device comprising;
(i) a substrate with at least one channel; wherein the channel has an inlet, an outlet and an internal space with a surface between the inlet and the outlet;
(ii) a porous monolith within the internal space of the channel, wherein the porous monolith is embedded with a plurality carbon nanotubes.

2. The microfluidic device of claim 1, wherein the channel is a straight line.

3. The microfluidic device of claim 1, wherein the channel is selected from the shape from the following group of channel shapes; a serpentine-shaped channel, a wedge shaped channel, a curved shaped channel between the inlet and the outlet.

4. The microfluidic device of claim 1, wherein the carbon particles are carbon nanotubes.

5. The microfluidic device of claim 1, wherein the carbon nanotubes are between 1-20 microns long.

6. The microfluidic device of claim 1, wherein the carbon nanotubes are about 5-15 microns long.

7. The microfluidic device of claim 1, wherein the carbon nanotubes are longer than 20 microns.

8. The microfluidic device of claim 1, wherein the carbon nanotubes are less than 100 microns in diameter.

9. The microfluidic device of claim 1, wherein the carbon nanotubes are greater than 100 microns in diameter.

10. The microfluidic device of claim 1, wherein the carbon nanotubes are less than 90 microns in diameter.

11. The microfluidic device of claim 1, wherein the carbon nanotubes are 90, 80, 70, 60, 50, 40, 30, 20 and 10 microns in diameter.

12. The microfluidic device of claim 1, further comprising a solid-phase extraction column, wherein the inlet of the solid-phase extraction column is connected to the outlet of the channel comprising the monolith embedded with carbon particles, and wherein a sample can be passed through channel comprising the carbon embedded monolith to the solid-phase extraction column.

13. The microfluidic device of any of the above claims, further comprising a filter membrane, wherein a outlet of the filter membrane is connected to the inlet of the inlet of the channel comprising the monolith embedded with carbon particles, and wherein a sample can be passed through the filter membrane prior to the channel comprising the carbon embedded monolith.

14. The microfluidic device of claim 13, wherein the elutant which has been through the filter membrane is passed through the channel comprising the carbon embedded monolith.

15. The microfluidic device of claim 13, wherein the sample which has collected on the filter membrane is passed through the channel comprising the carbon embedded monolith.

16. The microfluidic device of any of claims 12 to 15, wherein solid-phase extraction column comprises a silica bead and polymer composite.

17. The microfluidic device of claim 1, wherein the substrate comprises glass.

18. The microfluidic device of claim 1, wherein the substrate comprises plastic.

19. The microfluidic device of claim 1, wherein the substrate comprises metal.

20. The microfluidic device of claim 1, wherein the substrate comprises silica.

21. A method for bacterial lysis, the method comprising:
(i) suspending the bacteria in a suspension buffer;
(ii) passing the bacteria through a plurality of carbon nanotubes;
wherein the plurality of carbon nanotubes contact the bacteria and lyse the bacteria.

22. A method for bacterial lysis and DNA extraction in a single step, the method comprising:
(i) suspending the bacteria in a suspension buffer,
(ii) passing the bacteria through a plurality of carbon nanotubes; and
(iii) passing the bacteria from step (ii) through a solid-phase extraction column
wherein the plurality of carbon nanotubes and the solid-phase extraction column are located on a solid support.

23. The method of claim 21 or 22, wherein the suspension buffer is a chaotropic buffer.

24. The method of claim 23, wherein the suspension buffer further comprises at least one detergent.

25. The method of claim 21 or 22, wherein the bacteria is passed through the carbon nanotubes under pressure.

26. The method of claim 21 or 22, wherein the plurality of carbon nanotubes are present embedded in a monolith.

27. The method of claim 25, wherein the monolith embedded with carbon nanotubes is a polymer monolith embedded with carbon nanotubes.

28. The method of claim 21 or 22, wherein the carbon nanotubes are between 1-20 microns long.

29. The method of claim 21 or 22, wherein the carbon nanotubes are about 5-15 microns long.

30. The method of claim 21 or 22, wherein the carbon nanotubes are longer than 20 microns.

31. The method of claim 21 or 22, wherein the carbon nanotubes are less than 100 microns in diameter.

32. The method of claim 21 or 22, wherein the carbon nanotubes are greater than 100 microns in diameter.

33. The method of claim 31, wherein the carbon nanotubes are less than 90 microns in diameter.

34. The method of claim 31, wherein the carbon nanotubes are selected from a group of carbon nanotubes consisting of carbon nanotubes of at least: 90, 80, 70, 60, 50, 40, 30, and 10 microns in diameter.

35. The method of claim 21 or 22, wherein the solid-phase extraction column comprises a silica bead and polymer composite.

36. The method of claim 22, wherein the solid support is a chip.

37. The method of claim 36, wherein the chip comprises glass.

38. The method of claim 36, wherein the chip comprises plastic.

39. The method of claim 36, wherein the chip comprises metal.

40. The method of claim 36, wherein the chip comprises silica.

41. The method of claim 21 or 22, wherein the bacteria is gram-negative bacteria.

42. The method of claim 41, wherein the gram negative bacteria is E. Coli.

43. The method of claim 21 or 22, wherein the bacterial is gram-positive bacteria.

44. The method of claim 43, wherein the gram positive bacteria is B. subtillis or C. Difficile.

45. A method for obtaining nucleic acids from a cell using the device of any of claims 1 to 20.

46. The method of claim 45, wherein the cell is a bacterial cell.

47. The method of claim 46, wherein the bacteria is gram-negative bacteria.

48. The method of claim 47, wherein the gram negative bacteria is E. Coli.

49. The method of claim 46, wherein the bacterial is gram-positive bacteria.

50. The method of claim 49, wherein the gram positive bacteria is B. subtillis or C. Difficile.

51. The method of claim 45, wherein the nucleic acid is DNA.

52. The method of claim 45, wherein the nucleic acid is RNA.

53. The method of claim 45, wherein the sample is passed through the device of claim 23 under pressure.

54. The method of claim 43, wherein the pressure is applied through a syringe.

55. The method of claim 45, wherein the cell is suspended in a suspension buffer.

56. The method of claim 55, wherein the suspension buffer is a chaotropic buffer

57. The method of claim 56, wherein the suspension buffer further comprises at least one detergent.

58. The use of the microfluidic device of claims 1 to 20 for lysis of cells.

59. The use of the microfluidic device of claim 12 or 13 for obtaining nucleic acid from cells.

60. The use of the microfluidic device according to claim 58 or 59, wherein the cell is bacteria.

61. The use of the microfluidic device according to claim 60, wherein the bacteria is a gram-negative bacteria.

62. The use of the microfluidic device according to claim 61, wherein the gram negative bacteria is E. Coli.

63. The use of the microfluidic device according to claim 60, wherein the bacteria is gram-positive bacteria.

64. The use of the microfluidic device according to claim 63, wherein the gram positive bacteria is B. subtillis or C. Difficile.

65. The use of the microfluidic device according to claim 59, wherein the nucleic acid is DNA.

66. The use of the microfluidic device according to claim 59, wherein the nucleic acid is RNA.
